(19) United States
(12) Patent Application Publication
(10) Pub. No.: US 2024/0026391 A1
(43) Pub. Date: Jan. 25, 2024

(54) METHODS AND CELLS FOR PRODUCTION OF VOLATILE COMPOUNDS

(71) Applicant: Danmarks Tekniske Universitet, Kongens Lyngby (DK)

(72) Inventors: Ivan Pogrebnyakov, Kongens Lyngby (DK); Alex Toftgaard Nielsen, Kongens Lyngby (DK)

(21) Appl. No.: 18/043,177

(22) PCT Filed: Sep. 1, 2021
(86) PCT No.: PCT/EP2021/074134

§ 371 Date: Feb. 27, 2023

(30) Foreign Application Priority Data
Sep. 1, 2020 (EP) 20193767.9

(51) Int. Cl. C12P 7/28; C12P 7/04; C12N 9/10; C12N 9/16; C12N 9/88; C12N 9/04; C12N 15/74

(52) U.S. Cl. CPC C12P 7/28; C12P 7/04; C12N 9/1029; C12N 9/13; C12N 9/16; C12N 9/88; C12N 9/0006; C12N 15/74; C12Y 203/01009; C12Y 208/03008; C12Y 208/03009; C12Y 208/03005; C12Y 401/01004; C12Y 101/0108

(57) ABSTRACT

The present invention relates to thermophilic cells and methods for the microbial production of volatile compounds, including acetone, butanone and isopropanol. Also provided are nucleic acid constructs, vectors and host cells useful in such methods.

### TECHNICAL FIELD

The present invention relates to thermophilic cells and methods for the microbial production of volatile compounds, including acetone, butanone and isopropanol. Also provided are nucleic acid constructs, vectors and host cells useful in such methods.

### BACKGROUND

Acetone production via fermentation in Clostridium acetobutylicum was started at an industrial scale to meet the military needs during World War I. The technology spread rapidly and in the course of first half of the twentieth century it ranked in importance second only to ethanol fermentation. With the development of petrochemical industry acetone fermentation declined in the West, but some countries continued to use it up to 1980s and 90s.

Current concerns about the environmental impact of the use of petroleum and its depletion drive the search for alternative ways of chemicals production and has revived the interest in biological production of acetone. Acetone consumption in 2014 was 5.9 million tons and projected to grow up to 7.2 million tons by 2020 at a rate of about 3% annually (“Acetone market: global industry analysis and opportunity assessment, 2014-2020,” 2015). Today the vast majority of acetone is produced chemically via the cumene process. This is accompanied by significant environmental costs.

The native clostridial acetone pathway consists of three enzymatic steps starting from acetyl-CoA and acetate (FIG. 7). Whereas previous studies have addressed different aspects of acetone biosynthesis in the native host, Clostridium acetobutylicum (Jones et al., 1986), more recent studies took steps towards more advanced metabolic engineering in other organisms. In 1998 Bermejo et al. cloned the native acetone pathway from C. acetobutylicum into Escherichia coli and achieved yields comparable to and even higher than in the natural producer (Bernejo et al., 1998). Others modified the native pathway by introducing a hydrolysis reaction at the second step, which yields acetoacetate and CoA-SH (May et al., 2013). Others have modified metabolic network of E. coli by building synthetic pathways, e.g. the recently invented non-oxidative glycolysis (Bogorad et al., 2013; Yang et al., 2016) and part of the mevalonate pathway (Baer et al., 2016). Acetone is a relatively cheap commodity chemical. To make its biological production competitive with the petrochemical one, it is worth considering alternative production hosts, for example those that utilize feedstocks cheaper than refined sugars. For example the native pathway from C. acetobutylicum was expressed in a cyanobacterium which was able to produce acetone from CO₂ and water (Zhou et al., 2012). Other hosts for heterologous acetone production were C. ljungdahlii (Banerjee et al., 2014) and Acetobacterium woodii (Hoffmeister et al., 2016), which are both acetogens capable of metabolizing CO or the mixture of CO₂ and H₂ (synthesis gas). However, there remains also an interest in utilizing other crude and low cost carbon sources.

Representatives of the genus Geobacillus are also increasingly used as hosts for the production of chemicals (Bosma et al., 2013). Advantages of thermophilic production in Geobacillus include: 1) reduced risk of contamination by mesophiles; 2) higher reaction rates at elevated temperatures; 3) reduction of energy input for cooling of thermally pre-treated biomass; 4) ability of of geobacilli to utilize a wide range of carbon sources, including C6 and C5 sugars, and acetate. Additionally, acetone and other volatile compounds are evaporating at high temperatures and can be collected downstream, which facilitates their purification, at the same time reducing issues with product toxicity and product inhibition.

To date, most efforts on metabolic engineering have been focused on enhancing the production of Geobacillus' own fermentation by-products, notably ethanol. This was done by knocking out genes from competitive pathways and upregulating pathways, which led to increased fluxes towards ethanol (Cripps et al., 2009; Zhou et al., 2016).

One of the strategies to achieve higher acetone yields would be to construct alternative biosynthetic pathways.

There thus remains a need for cells and methods allowing biological production of acetone, butanone and isopropanol in an efficient, cost-effective and sustainable manner.

### SUMMARY

Herein is provided a method of producing one or more compounds selected from acetone, butanone and isopropanol, said method comprising the steps of:
a) Providing a thermophilic cell, preferably a thermophilic bacterial or a thermophilic archaeal cell expressing:
   i) a first enzyme selected from:
      an acetyl-CoA acetyltransferase (EC 2.3.1.9) selected from GHH_c20420 (SEQ ID NO: 1), Slip_0499 (SEQ ID NO: 2), Caur_1461 (SEQ ID NO: 3), Slip_0479 (SEQ ID NO: 4), Tfu_1520 (SEQ ID NO: 5), Tfu_0436 (SEQ ID NO: 6), Slip_0880 (SEQ ID NO: 7), Tfu_2394 (SEQ ID NO: 8), Slip_1236 (SEQ ID NO: 9), Caur 1540 (SEQ ID NO: 10), Tfu_0253 (SEQ ID NO: 11), CHY_1604 (SEQ ID NO: 14), CHY_1288 (SEQ ID NO: 15), Slip_2085 (SEQ ID NO: 16), Slip_0465 (SEQ ID NO: 17), Dde1 (SEQ ID NO: 59), Rxy2 (SEQ ID NO: 60) and CHY_1355 (SEQ ID NO: 18),
      an enzyme of EC number 2.3.3.20 selected from the 3-oxoacyl-ACP synthase SVA_3859 (SEQ ID NO: 12) and the acyl-CoA:acyl-CoA alkyltransferase Despr_2661 (SEQ ID NO: 13); and
      functional variants thereof having at least 70% homology, similarity or identity thereto;
   ii) a second enzyme selected from an acetate CoA transferase, a 3-oxoacid CoA transferase, an acyl CoA:acetate/3-ketoacid CoA-transferase, and an acyl-CoA thioesterase II,
      wherein the second enzyme is selected from: Tle2, Dde2 (EC 2.8.3.5) (SEQ ID NO: 21), Ghh2 (EC 2.8.3.5), Tme (EC 2.8.3.8), Pth (EC 2.8.3.1) (SEQ ID NO: 26) and Rma (EC 3.1.2.-) (SEQ ID NO: 27), or functional variants thereof having at least 70% homology, similarity or identity thereto,
      wherein Tle2 consists of Tle2 subunit A (EC 2.8.3.8) (SEQ ID NO: 19) and Tle2 subunit B (EC 2.8.3.9) (SEQ ID NO: 20), wherein Ghh2 consists of Ghh2 subunit A (SEQ ID NO: 22) and Ghh2 subunit B (SEQ ID NO: 23), and wherein Tme consists of Tme subunit A (SEQ ID NO: 24) and Tme subunit B (SEQ ID NO: 25), or functional variants thereof having at least 70% homology, similarity or identity thereto,
      and
   iii) an acetoacetate decarboxylase (EC 4.1.1.4), preferably Cac (SEQ ID NO: 28) or a functional variant thereof having at least 70% homology, similarity or identity thereto;
   iv) optionally an isopropanol dehydrogenase (EC 1.1.1.80), preferably Tbr (SEQ ID NO: 29) or a functional variant thereof having at least 70% homology, similarity or identity thereto;
b) cultivating the bacterial cell in a bioreactor comprising a cultivation broth at a temperature of between 42 and 80° C., such as between 50 and 75° C., for example at 60° C., thereby producing the one or more compounds;
c) recovering the one or more compounds produced during step b).

Herein is also provided a thermophilic cell capable of producing acetone and/or butanone and optionally isopropanol, said cell being a bacterial cell or an archaeal cell and expressing:
i) a first enzyme selected from:
   an acetyl-CoA acetyltransferase (EC 2.3.1.9) selected from GHH_c20420 (SEQ ID NO: 1), Slip_0499 (SEQ ID NO: 2), Caur_1461 (SEQ ID NO: 3), Slip_0479 (SEQ ID NO: 4), Tfu_1520 (SEQ ID NO: 5), Tfu_0436 (SEQ ID NO: 6), Slip_0880 (SEQ ID NO: 7), Tfu_2394 (SEQ ID NO: 8), Slip_1236 (SEQ ID NO: 9), Caur 1540 (SEQ ID NO: 10), Tfu_0253 (SEQ ID NO: 11), CHY_1604 (SEQ ID NO: 14), CHY_1288 (SEQ ID NO: 15), Slip_2085 (SEQ ID NO: 16), Slip_0465 (SEQ ID NO: 17), Dde1 (SEQ ID NO: 59), Rxy2 (SEQ ID NO: 60) and CHY_1355 (SEQ ID NO: 18), or
   an enzyme of EC number 2.3.3.20 selected from the 3-oxoacyl-ACP synthase SVA_3859 (SEQ ID NO: 12) and the acyl-CoA:acyl-CoA alkyltransferase Despr_2661 (SEQ ID NO: 13); and
   functional variants thereof having at least 70% homology, similarity or identity thereto;
ii) a second enzyme selected from an acetate CoA transferase, a 3-oxoacid CoA transferase, an acyl CoA:acetate/3-ketoacid CoA-transferase, and an acyl-CoA thioesterase II
   wherein the second enzyme is selected from: Tle2, Dde2 (EC 2.8.3.5) (SEQ ID NO: 21), Ghh2 (EC 2.8.3.5), Tme (EC 2.8.3.8), Pth (EC 2.8.3.1) (SEQ ID NO: 26) and Rma (EC 3.1.2.-) (SEQ ID NO: 27), or functional variants thereof having at least 70% homology, similarity or identity thereto,
   wherein Tle2 consists of Tle2 subunit A (EC 2.8.3.8) (SEQ ID NO: 19) and Tle2 subunit B (EC 2.8.3.9) (SEQ ID NO: 20), wherein Ghh2 consists of Ghh2 subunit A (SEQ ID NO: 22) and Ghh2 subunit B (SEQ ID NO: 23), and wherein Tme consists of Tme subunit A (SEQ ID NO: 24) and Tme subunit B (SEQ ID NO: 25), and
iii) an acetoacetate decarboxylase (EC 4.1.1.4), preferably Cac (SEQ ID NO: 28) or a functional variant thereof having at least 70% homology, similarity or identity thereto;
whereby the cell can convert acetyl-CoA to acetone, thereby producing acetone with a titer of at least 0.8 g/L;
and/or whereby the cell can convert acetyl-CoA and propionyl-CoA to butanone, thereby producing butanone;
iv) optionally an isopropanol dehydrogenase (EC 1.1.1.80), preferably Tbr (SEQ ID NO: 29) or a functional variant thereof having at least 70% homology, similarity or identity thereto,
   whereby the cell can further convert acetone to isopropanol, thereby producing isopropanol.

Herein is also provided a nucleic acid construct for modifying a thermophilic cell selected from a thermophilic bacterial cell and a thermophilic archaeal cell, comprising:
i) a polynucleotide encoding an acetyl-CoA acetyltransferase (EC 2.3.1.9) or a functional variant thereof having at least 70% homology, similarity or identity thereto, wherein the acetyl-CoA acetyltransferase is selected from GHH_c20420 (SEQ ID NO: 1), Slip_0499 (SEQ ID NO: 2), Caur_1461 (SEQ ID NO: 3), Slip_0479 (SEQ ID NO: 4), Tfu_1520 (SEQ ID NO: 5), Tfu_0436 (SEQ ID NO: 6), Slip 0880 (SEQ ID NO: 7), Tfu 2394 (SEQ ID NO: 8), Slip 1236 (SEQ ID NO: 9), Caur_1540 (SEQ ID NO: 10), Tfu_0253 (SEQ ID NO: 11), CHY_1604 (SEQ ID NO: 14), CHY_1288 (SEQ ID NO: 15), Slip_2085 (SEQ ID NO: 16), Slip_0465 (SEQ ID NO: 17), Dde1 (SEQ ID NO: 59), Rxy2 (SEQ ID NO: 60) and CHY_1355 (SEQ ID NO: 18), and/or an enzyme of EC number 2.3.3.20 selected from the 3-oxoacyl-ACP synthase SVA_3859 (SEQ ID NO: 12) and the acyl-CoA:acyl-CoA alkyltransferase Despr_2661 (SEQ ID NO: 13);
ii) a polynucleotide encoding a second enzyme selected from an acetate CoA transferase, a 3-oxoacid CoA transferase, an acyl CoA:acetate/3-ketoacid CoA-transferase, and an acyl-CoA thioesterase II
   wherein the second enzyme is selected from: Tle2, Dde2 (EC 2.8.3.5) (SEQ ID NO: 21), Ghh2 (EC 2.8.3.5), Tme (EC 2.8.3.8), Pth (EC 2.8.3.1) (SEQ ID NO: 26) and Rma (EC 3.1.2.-) (SEQ ID NO: 27), or functional variants thereof having at least 70% homology, similarity or identity thereto,
   wherein Tle2 consists of Tle2 subunit A (EC 2.8.3.8) (SEQ ID NO: 19) and Tle2 subunit B (EC 2.8.3.9) (SEQ ID NO: 20), wherein Ghh2 consists of Ghh2 subunit A (SEQ ID NO: 22) and Ghh2 subunit B (SEQ ID NO: 23), and wherein Tme consists of Tme subunit A (SEQ ID NO: 24) and Tme subunit B (SEQ ID NO: 25), or functional variants thereof having at least 70% homology, similarity or identity thereto, and
iii) a polynucleotide encoding an acetoacetate decarboxylase (EC 4.1.1.4) or a functional variant thereof having at least 70% homology, similarity or identity thereto, preferably Cac (SEQ ID NO: 28).

Herein is also provided a vector comprising the nucleic acid construct disclosed herein.

Herein is also provided a thermophilic cell comprising the nucleic acid construct and/or the vector disclosed herein, wherein the thermophilic cell is a thermophilic bacterial cell or a thermophilic archaeal cell.

Herein is also provided a kit comprising the nucleic acid construct, the vector or the thermophilic cell described herein.

### DESCRIPTION OF THE DRAWINGS

FIG. 1: G. thermoglucosidasius as a host for acetone production. (A) Acetone tolerance. Cells were grown at different concentrations of acetone and final densities were measured. (B) Biosynthesis of acetone using three enzyme combinations at different conditions. Each letter stands for an enzyme, position corresponds to the enzymatic step in the pathway. Enzyme sources: D: D. desulfuricans; G: Geobacillus sp. GHH01; C: C. acetobutylicum

FIG. 2: Production of butanone and acetone in G. thermoglucosidasius expressing the indicated thiolase, and otherwise expressing the acetyl CoA transferase Tle2 from Pseudothermotoga lettingae (UniProt ID A8F7H7, A8F7H6) and the acetoacetate decarboxylase Cac from Clostridium acetobutylicum (P23670).

FIG. 3: Correlation between acetone production by Dde1-Dde2-Cac operon and the strength of promoters which drive its expression in G. thermoglucosidasius.

FIG. 4: Production of acetone by G. thermoglucosidasius strain CTC in TMM medium with 1% glucose and different concentrations of acetate (data from Table 4).

FIG. 5: Production of acetone by G. thermoglucosidasius strain CTC in TMM medium with 0.2% acetic acid different concentrations of glucose and xylose.

FIG. 6: Production of butanone by G. thermoglucosidasius strain CTC in semi-defined medium TMM with 1% glucose and different concentrations of propionic acid (data from Table 6).

FIG. 7: Native acetone pathway in Clostridium acetobutylicum.

FIG. 8: Production of butanone by G. thermoglucosidasius strain CTC in semi-defined medium TMM with 2% glucose, 0.2% acetic acid and 1% yeast extract, 30 L fed-batch fermentation: 2 g/L/h glucose, 1 g/L/h acetic acid, 1 g/L/h yeast extract. X-axis: time in hours; left Y-axis: acetone, g/L; right Y-axis: CO₂, g/L.

FIG. 9: Production of acetone in G. thermoglucosidasius expressing the indicated thiolase, and otherwise expressing the acetyl CoA transferase Tle2 from Pseudothermotoga lettingae (UniProt ID A8F7H7, A8F7H6) and the acetoacetate decarboxylase Cac from Clostridium acetobutylicum (P23670). Y-axis: acetone, mg/L.

FIG. 10: Production of acetone in the STC strain (Slip_0880-Tle2-Cac) and in the CTC strain (Caur_1461-Tle2-Cac) in a 1 L constant fed-batch fermentation. The strains were grown in TMM medium, supplemented with 2% glucose, 0.2% acetic acid, 1% yeast extract. Y-axis: acetone, g/L. X-axis: time, hours.

### DETAILED DESCRIPTION OF THE INVENTION

The present disclosure relates to methods for microbial production of volatile compounds, in particular acetone, butanone and isopropanol. By taking advantage of thermophilic cells for production, these compounds can be easily and continuously removed from a fermentation, such as a fermentation broth, which, in addition to rendering the process labour- and cost-efficient, also solves the problem of product inhibition and negative effects on growth associated to the toxicity of the product(s). Other advantages include reduced risks of contamination due to the relatively high production temperatures.

### Definitions

The term “thermophile” herein refers to microorganisms, in particular bacteria and archaea, that thrive best, or at least capable of growing, at temperatures above 42° C.

Functional variant: the term is herein applied to functional variants of enzymes, i.e. modified versions of an enzyme, or homologous enzymes originating from a different species, which retain some or all the catalytic activity of the original enzyme. Functional variants may have been modified by introducing mutations which confer e.g. increased activity, a change in intracellular localisation, increased thermostability, prolonged half-life, among others, but retain the ability to perform the same enzymatic reaction as the enzymes they are derived from, albeit possibly to a different extent. Preferably the mutation(s) introduced in the functional variant are mutations in the gene encoding the corresponding enzyme, for example a mutation in the promoter of the gene or in the coding sequence encoding the enzyme.

“Identity”, “similarity” and “homology” with respect to a polynucleotide (or polypeptide) is defined herein as the percentage of nucleic acids (or amino acids) in the candidate sequence that are identical with the residues of a corresponding native nucleic acids (or amino acids), after aligning the sequences and introducing gaps, if necessary, to achieve the maximum percent identity/similarity/homology, and considering any conservative substitutions according to the NCIUB rules (hftp://www.chem.qmul.ac.uk/iubmb/misc/naseq.html; NC-IUB, Eur J Biochem (1985) 150: 1-5) as part of the sequence identity. Neither 5′ or 3′ extensions nor insertions (for nucleic acids) or N′ or C′ extensions nor insertions (for polypeptides) result in a reduction of identity, similarity or homology. Methods and computer programs for the alignments are well known in the art. Generally, a given homology between two sequences implies that the identity between these sequences is at least equal to the homology; for example, if two sequences are 70% homologous to one another, they cannot be less than 70% identical to one another—but could be sharing 80% identity. Throughout this disclosure, a sequence (amino acid sequence or nucleic acid sequence) sharing at least 70% identity, homology or similarity to another sequence means that the sequence shares at least 70% identity, homology or similarity to said sequence, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% identity, homology or similarity.

The term “acetyl-CoA acetyltransferase” or “thiolase” herein refers to an enzyme that catalyzes either the conversion of two molecules of acetyl-CoA to acetoacetyl-CoA and coenzyme A (CoA), or the conversion of one acetyl-CoA and one propionyl-CoA leading to 3-ketovaleryl-CoA. In particular the term refers to acetyl-CoA acetyltransferases of EC number 2.3.1.9. These particular enzymes have a substrate preference for acetyl-CoA or propionyl-CoA and therefore preferably catalyse the reaction in the forward direction. The skilled person will know how to determine whether a mutant enzyme has thiolase activity. For example, the potential thiolase can be incubated with acetoacetyl-CoA and CoA, and absorbance at 303 nm can be monitored. A decrease in the absorbance at 303 nm indicates that the potential thiolase can perform said reaction and has thiolase activity.

The term “3-oxoacyl-ACP synthase” (3-oxoacyl-[acyl-carrier-protein] synthase) and “acyl-CoA:acyl-CoA alkyltransferase” refers to the same enzymes of EC number 2.3.3.20. They catalyse the conversion of two molecules of acyl-CoA into one molecule of (2R)-2-alkyl-3-oxoalkanoate in the presence of water, thereby generating two molecules of coenzyme A (CoA). The reaction is a head-to-head non-decarboxylative Claisen condensation. The skilled person will know how to determine whether a mutant enzyme has 3-oxoacyl-ACP synthase activity. For example, the potential 3-oxoacyl-ACP synthase can be incubated with acetyl-CoA (or acetyl-CoA and propionyl-CoA), with subsequent addition of 5,5′-dithio-bis-(2-nitrobenzoic acid), which reacts with a free thiol group of the released CoASH. The absorbance of the product can be monitored at 412 nm. Formation of the product indicates that the potential 3-oxoacyl-ACP synthase retains 3-oxoacyl-ACP synthase activity.

Acetoacetate decarboxylase: The term herein refers to is an enzyme of EC number 4.1.1.4. Acetoacetate decarboxylases are involved in both the ketone body production pathway in humans and other mammals, and solventogenesis in bacteria. They catalyse the decarboxylation of acetoacetate, yielding acetone and carbon dioxide. The skilled person will know how to determine whether a mutant enzyme has acetoacetate decarboxylase activity. For example, the potential acetoacetate decarboxylase can be incubated with lithium acetoacetate. The accompanying release of CO₂ which ensues can be monitored, e.g. manometrically. Release of CO₂ indicates that the tested enzyme has acetoacetate decarboxylase activity.

Acetate CoA-transferase (EC 2.8.3.8) is an enzyme that catalyzes the chemical reaction: acyl-CoA+an acetate [symbol] fatty acid+an acetyl-CoA. The activity of variants of acetate CoA transferase can be measured by methods known in the art, for example by incubating this enzyme with acetyl-CoA and lithium acetoacetate, and following the acetoacetyl-CoA formation by measuring absorbance at 313 nm. 3-oxoacid CoA-transferase (EC 2.8.3.5) is an enzyme which catalyzes the chemical reaction 3-ketovaleryl-CoA+fatty acid [symbol] 3-oxopentanoate+acyl-CoA. The activity of variants of acetate CoA transferase can be tested by methods known in the art, for example by incubating this enzyme with acetyl-CoA and lithium 3-oxopentanoate, and following the 3-ketovaleryl-CoA formation by measuring absorbance at 304 nm.

An acyl CoA:acetate/3-ketoacid CoA-transferase (EC 2.8.3.1) is an enzyme that catalyzes the chemical reaction 3-ketoacyl-CoA+fatty acid [symbol] 3-ketoacid+acyl-CoA. Other names include propionate CoA-transferase, acetyl-CoA:propanoate CoA-transferase, propionate coenzyme A-transferase, propionate-CoA:lactoyl-CoA transferase, propionyl CoA:acetate CoA transferase, and propionyl-CoA transferase. The activity of variants of acetate CoA transferase can be measured by methods known in the art, for example by incubating this enzyme with acetyl-CoA and lithium 3-oxopentanoate, and following the 3-ketovaleryl-CoA formation by measuring absorbance at 304 nm.

An acyl-CoA thioesterase II (EC 3.1.2.-) is an enzyme, which catalyzes the chemical reaction of hydrolysis: acyl-CoA+H₂O→fatty acid+CoA. The skilled person will know how to determine whether a mutant enzyme has acyl-CoA thioesterase II activity. For example, the potential acyl-CoA thioesterase II can be incubated with acetoacetyl-CoA in the presence of 5,5′-dithiobis(2-nitrobenzoic acid). The release of free thiol groups of CoA will result in formation of 5-thio-2-nitrobenzoate, which can be quantified by measuring the absorbance at 412 nm.

Isopropanol dehydrogenase: isopropanol dehydrogenase (NADP+) (EC 1.1.1.80) is an enzyme that catalyzes the conversion of propan-2-ol to acetone and acetone to propan-2-ol. The activity of (mutated) variants of isopropanol dehydrogenase can be measured by incubating this enzyme with acetone and NAD(P)H, and following the NAD(P)H oxidation by measuring absorbance at 340 nm.

Titer: the titer of a compound refers herein to the produced concentration of a compound. When the compound is produced by a cell, the term refers to the total concentration produced by the cell, i.e. the total amount of the compound divided by the volume of the culture medium. This means that, particularly for volatile compounds, the titer includes the portion of the compound which may have evaporated from the culture medium, and it is thus determined by collecting the produced compound from the fermentation broth and from potential off-gas from the fermenter.

Herein is provided a method of producing one or more compounds selected from acetone, butanone and isopropanol, said method comprising the steps of:
a) Providing a thermophilic cell, preferably a thermophilic bacterial or a thermophilic archaeal cell expressing:
   i) a first enzyme selected from:
      an acetyl-CoA acetyltransferase (EC 2.3.1.9) selected from GHH_c20420 (SEQ ID NO: 1), Slip_0499 (SEQ ID NO: 2), Caur 1461 (SEQ ID NO: 3), Slip_0479 (SEQ ID NO: 4), Tfu_1520 (SEQ ID NO: 5), Tfu_0436 (SEQ ID NO: 6), Slip_0880 (SEQ ID NO: 7), Tfu_2394 (SEQ ID NO: 8), Slip_1236 (SEQ ID NO: 9), Caur 1540 (SEQ ID NO: 10), Tfu_0253 (SEQ ID NO: 11), CHY_1604 (SEQ ID NO: 14), CHY_1288 (SEQ ID NO: 15), Slip_2085 (SEQ ID NO: 16), Slip_0465 (SEQ ID NO: 17), Dde1 (SEQ ID NO: 59), Rxy2 (SEQ ID NO: 60) and CHY_1355 (SEQ ID NO: 18), or
      an enzyme of EC number 2.3.3.20 selected from the 3-oxoacyl-ACP synthase SVA_3859 (SEQ ID NO: 12) and the acyl-CoA:acyl-CoA alkyltransferase Despr_2661 (SEQ ID NO: 13); and
      functional variants thereof having at least 70% homology, similarity or identity thereto;
   ii) a second enzyme selected from an acetate CoA transferase, a 3-oxoacid CoA transferase, an acyl CoA:acetate/3-ketoacid CoA-transferase, and an acyl-CoA thioesterase II
      wherein the second enzyme is selected from: Tle2, Dde2 (EC 2.8.3.5) (SEQ ID NO: 21), Ghh2 (EC 2.8.3.5), Tme (EC 2.8.3.8), Pth (EC 2.8.3.1) (SEQ ID NO: 26) and Rma (EC 3.1.2.-) (SEQ ID NO: 27), or functional variants thereof having at least 70% homology, similarity or identity thereto,
      wherein Tle2 consists of Tle2 subunit A (EC 2.8.3.8) (SEQ ID NO: 19) and Tle2 subunit B (EC 2.8.3.9) (SEQ ID NO: 20), wherein Ghh2 consists of Ghh2 subunit A (SEQ ID NO: 22) and Ghh2 subunit B (SEQ ID NO: 23), and wherein Tme consists of Tme subunit A (SEQ ID NO: 24) and Tme subunit B (SEQ ID NO: 25),
      and
   iii) an acetoacetate decarboxylase (EC 4.1.1.4), preferably Cac (SEQ ID NO: 28) or a functional variant thereof having at least 70% homology, similarity or identity thereto;
   iv) optionally an isopropanol dehydrogenase (EC 1.1.1.80), preferably Tbr (SEQ ID NO: 29) or a functional variant thereof having at least 70% homology, similarity or identity thereto;
b) cultivating the bacterial cell in a bioreactor comprising a cultivation broth at a temperature of between 42 and 80° C., such as between 50 and 75° C., for example at 60° C., thereby producing the one or more compounds;
c) recovering the one or more compounds produced during step b).

### Thermophilic Cell

The cells employed in the context of the present disclosure are thermophilic cells, more specifically bacterial or archaeal cells. In particular, bacterial or archaeal cells which have an optimal growth temperature of 42° C. or more are of interest. The term “cell” will herein generally, unless specified otherwise, be construed to designate thermophilic cells, more specifically thermophilic bacterial cells or thermophilic archaeal cells, i.e. cells which are capable of growing at temperatures of 42° C. or more.

Herein is provided a thermophilic cell capable of producing acetone and/or butanone and optionally isopropanol, said cell being a bacterial cell or an archaeal cell and expressing:
i) a first enzyme selected from:
   an acetyl-CoA acetyltransferase (EC 2.3.1.9) selected from GHH_c20420 (SEQ ID NO: 1), Slip_0499 (SEQ ID NO: 2), Caur 1461 (SEQ ID NO: 3), Slip_0479 (SEQ ID NO: 4), Tfu_1520 (SEQ ID NO: 5), Tfu_0436 (SEQ ID NO: 6), Slip_0880 (SEQ ID NO: 7), Tfu_2394 (SEQ ID NO: 8), Slip_1236 (SEQ ID NO: 9), Caur 1540 (SEQ ID NO: 10), Tfu_0253 (SEQ ID NO: 11), CHY_1604 (SEQ ID NO: 14), CHY_1288 (SEQ ID NO: 15), Slip 2085 (SEQ ID NO: 16), Slip 0465 (SEQ ID NO: 17), Dde1 (SEQ ID NO: 59), Rxy2 (SEQ ID NO: 60) and CHY_1355 (SEQ ID NO: 18), or
   an enzyme of EC number 2.3.3.20 selected from the 3-oxoacyl-ACP synthase SVA_3859 (SEQ ID NO: 12) and the acyl-CoA:acyl-CoA alkyltransferase Despr_2661 (SEQ ID NO: 13); and
   functional variants thereof having at least 70% homology, similarity or identity thereto;
ii) a second enzyme selected from an acetate CoA transferase, a 3-oxoacid CoA transferase, an acyl CoA:acetate/3-ketoacid CoA-transferase, and an acyl-CoA thioesterase II
   wherein the second enzyme is selected from: Tle2, Dde2 (EC 2.8.3.5) (SEQ ID NO: 21), Ghh2 (EC 2.8.3.5), Tme (EC 2.8.3.8), Pth (EC 2.8.3.1) (SEQ ID NO: 26) and Rma (EC 3.1.2.-) (SEQ ID NO: 27), or functional variants thereof having at least 70% homology, similarity or identity thereto, wherein Tle2 consists of Tle2 subunit A (EC 2.8.3.8) (SEQ ID NO: 19) and Tle2 subunit B (EC 2.8.3.9) (SEQ ID NO: 20), wherein Ghh2 consists of Ghh2 subunit A (SEQ ID NO: 22) and Ghh2 subunit B (SEQ ID NO: 23), and wherein Tme consists of Tme subunit A (SEQ ID NO: 24) and Tme subunit B (SEQ ID NO: 25), and
iii) an acetoacetate decarboxylase (EC 4.1.1.4), preferably Cac (SEQ ID NO: 28) or a functional variant thereof having at least 70% homology, similarity or identity thereto;
whereby the cell can convert acetyl-CoA to acetone, thereby producing acetone with a titer of at least 0.8 g/L;
and/or whereby the cell can convert acetyl-CoA and propionyl-CoA to butanone, thereby producing butanone;
iv) optionally an isopropanol dehydrogenase (EC 1.1.1.80), preferably Tbr (SEQ ID NO: 29) or a functional variant thereof having at least 70% homology, similarity or identity thereto,
   whereby the cell can further convert acetone to isopropanol, thereby producing isopropanol.

The present invention takes advantage of thermophilic cells for bioproduction of volatile compounds, in particular acetone, butanone and isopropanol. Because such cells thrive at higher temperatures than conventional, i.e. non-thermophilic, cells, recovery of the volatile products can be facilitated, as these are typically present in the off-gases produced during cultivation of the thermophilic cell. Not only does this reduce the production costs, it is also generally expected to be beneficial for the longevity of the producer as the end product (acetone, butanone and isopropanol) is typically toxic for the producing cell.

The thermophilic cells described herein have been engineered to produce volatile compounds, i.e. acetone, butanone and/or isopropanol. The thermophilic cells described therein preferably do not occur naturally. In some embodiments, the thermophilic cell is a non-natural cell or an engineered cell, which has been modified either to express a heterologous pathway, i.e. a pathway which is not present in the parent cell, or to express a modified native pathway.

In some embodiments, the thermophilic cell capable of producing acetone and/or butanone and optionally isopropanol is a bacterial cell or an archaeal cell and expresses:
i) a first enzyme selected from:
   an acetyl-CoA acetyltransferase (EC 2.3.1.9) selected from GHH_c20420 as set forth in SEQ ID NO: 1, Slip_0499 as set forth in SEQ ID NO: 2, Caur_1461 as set forth in SEQ ID NO: 3, Slip_0479 as set forth in SEQ ID NO: 4, Slip_0880 as set forth in SEQ ID NO: 7, and Dde1 as set forth in SEQ ID NO: 59, or functional variants thereof having at least 70% identity or similarity thereto;
ii) a second enzyme selected from an acetate CoA transferase, a 3-oxoacid CoA transferase, an acyl CoA:acetate/3-ketoacid CoA-transferase, and an acyl-CoA thioesterase II
   wherein the second enzyme is selected from: Tle2 and Dde2 (EC 2.8.3.5) as set forth in SEQ ID NO: 21, or functional variants thereof having at least 70% identity or similarity thereto,
   wherein Tle2 consists of Tle2 subunit A (EC 2.8.3.8) as set forth in SEQ ID NO: 19 and Tle2 subunit B (EC 2.8.3.9) as set forth in SEQ ID NO: 20, and
iii) an acetoacetate decarboxylase (EC 4.1.1.4), wherein the acetoacetate decarboxylase is Cac as set forth in SEQ ID NO: 28 or a functional variant thereof having at least 70% identity or similarity thereto;
   whereby the cell can convert acetyl-CoA to acetone, thereby producing acetone with a titer of at least 0.8 g/L;
   and/or whereby the cell can convert acetyl-CoA and propionyl-CoA to butanone, thereby producing butanone;
   and
iv) optionally an isopropanol dehydrogenase (EC 1.1.1.80), wherein the isopropanol dehydrogenase is Tbr as set forth in SEQ ID NO: 29 or a functional variant thereof having at least 70% identity or similarity thereto,
   whereby the cell can further convert acetone to isopropanol, thereby producing isopropanol.

In some embodiments, the thermophilic cell is a bacterial cell, i.e. a cell of a thermophilic bacterium. In other embodiments, the thermophilic cell is an archaeal cell, i.e. a cell of a thermophilic archaeon.

In some embodiments, the thermophilic cell belongs to a genus selected from Geobacillus, Thermoanaerobacterium, Thermoanaerobacter, Caldanaerobacter, Bacillus, Thermoclostridium, Anoxybacillus, Caldicellulosiruptor, Moorella, Thermus, Thermotoga, Pseudothermotoga, Chloroflexus, Anaerocellum, Rhodothermus, Sulfolobus, Thermococcus, Pyrococcus and Clostridium. In specific embodiments, the thermophilic cell is a Geobacillus cell, a Bacillus cell or a Clostridium cell.

In some embodiments, the thermophilic cell belongs to a species selected from Geobacillus thermoglucosidasius, Geobacillus toebii, Geobacillus stearothermophilus, Geobacillus thermodenitrificans, Geobacillus kaustophilus, Geobacillus thermoleovorans, Geobacillus thermocatenulatus, Thermoanaerobacterium xylanolyticum, Thermoanaerobacterium saccharotyticum, Thermoanaerobacterium thermosaccharolyticum, Thermoanaerobacter mathranii, Thermoanaerobacter pseudoethanolicus, Thermoanaerobacter brockii, Thermoanaerobacter kivui, Thermoanaerobacter brockii, Caldanaerobacter subterraneus, Clostridium thermocellum, Clostridium thermosuccinogenes, Thermoclostridium stercorarium, Bacillus subtilis, Bacillus licheniformis, Bacillus coagulans, Bacillus smithii, Bacillus methanolicus, Bacillus flavothermus, Anoxybacillus kamchatkensis, Anoxybacillus gonensis, Caldicellulosiruptor bescii, Caldicellulosiruptor saccharolyticus, Caldicellulosiruptor kristjanssonii, Caldicellulosiruptor owensensis, Caldicellulosiruptor lactoaceticus, Moorella thermoacetica, Moorella thermoautotrophica, Thermus thermophilus, Thermus aquaticus, Thermotoga maritima, Pseudothermotoga lettingae, Pseudothermotoga thermarum, Chloroflexus aurantiacus, Anaerocellum thermophilum, Rhodothermus marinus, Sulfolobus acidocaldarius, Sulfolobus islandicus, Sulfolobus solfataricus, Thermococcus barophilus, Thermococcus kodakarensis, Pyrococcus abyssi, and Pyrococcus furiosus. In specific embodiments, the cell is a Geobacillus thermoglucosidasius cell. In other embodiments, the cell is a Bacillus subtilis cell. In other embodiments, the cell is a Clostridium thermocellum cell.

In some embodiments, the thermophilic cell has an optimal growth temperature between 42 and 80° C., or is capable of growing at a temperature between 42 and 80° C., such as between 50 and 75° C., for example at 60° C. For example, the thermophilic cell has an optimal growth temperature of 42° C. or more, such as 43° C. or more, such as 44° C. or more, such as 45° C. or more, such as 46° C. or more, such as 47° C. or more, such as 48° C. or more, such as 49° C. or more, such as 50° C. or more, such as 51° C. or more, 52° C. or more, 53° C. or more, 54° C. or more, 55° C. or more, 56° C. or more, 57° C. or more, 58° C. or more, 59° C. or more, for example 60° C. or more. In some embodiments, the thermophilic cell is capable of growing at a temperature of between 42 and 80° C., such as between 50 and 75° C., for example at 60° C. For example, the thermophilic cell is capable of growing at a temperature of 42° C. or more, such as 43° C. or more, such as 44° C. or more, such as 45° C. or more, such as 46° C. or more, such as 47° C. or more, such as 48° C. or more, such as 49° C. or more, such as 50° C. or more, such as 51° C. or more, 52° C. or more, 53° C. or more, 54° C. or more, 55° C. or more, 56° C. or more, 57° C. or more, 58° C. or more, 59° C. or more, for example 60° C. or more.

In particular, the thermophilic cell is preferably able to grow at temperatures where at least part of the acetone, butanone and/or isopropanol it produces evaporates, thereby facilitating recovery of the produced acetone, butanone and/or isopropanol. Thus, in some embodiments, the thermophilic cell is able to grow at temperatures equal to or greater than the boiling point of acetone, butanone and/or isopropanol. In some embodiments, the thermophilic cell is capable of growing at a temperature of 56° C. (boiling point of acetone) or more.

### Production of Volatile Compounds

Herein are disclosed methods and cells useful for producing volatile compounds, in particular one or more compounds selected from acetone, butanone and isopropanol.

The thermophilic cells disclosed herein express the enzymes necessary to achieve production of said compounds.

Herein is thus provided a method of producing one or more compounds selected from acetone, butanone and isopropanol, said method comprising the steps of:
a) Providing a thermophilic cell, preferably a thermophilic bacterial or a thermophilic archaeal cell expressing:
   i) a first enzyme selected from:
      an acetyl-CoA acetyltransferase (EC 2.3.1.9) selected from GHH_c20420 (SEQ ID NO: 1), Slip_0499 (SEQ ID NO: 2), Caur 1461 (SEQ ID NO: 3), Slip_0479 (SEQ ID NO: 4), Tfu_1520 (SEQ ID NO: 5), Tfu_0436 (SEQ ID NO: 6), Slip_0880 (SEQ ID NO: 7), Tfu_2394 (SEQ ID NO: 8), Slip_1236 (SEQ ID NO: 9), Caur 1540 (SEQ ID NO: 10), Tfu_0253 (SEQ ID NO: 11), CHY_1604 (SEQ ID NO: 14), CHY_1288 (SEQ ID NO: 15), Slip_2085 (SEQ ID NO: 16), Slip_0465 (SEQ ID NO: 17), Dde1 (SEQ ID NO: 59), Rxy2 (SEQ ID NO: 60) and CHY_1355 (SEQ ID NO: 18),
      an enzyme of EC number 2.3.3.20 selected from the 3-oxoacyl-ACP synthase SVA_3859 (SEQ ID NO: 12) and the acyl-CoA:acyl-CoA alkyltransferase Despr_2661 (SEQ ID NO: 13); and
      functional variants thereof having at least 70% homology, similarity or identity thereto;
   ii) a second enzyme selected from an acetate CoA transferase, a 3-oxoacid CoA transferase, an acyl CoA:acetate/3-ketoacid CoA-transferase, and an acyl-CoA thioesterase II,
      wherein the second enzyme is selected from: Tle2, Dde2 (EC 2.8.3.5) (SEQ ID NO: 21), Ghh2 (EC 2.8.3.5), Tme (EC 2.8.3.8), Pth (EC 2.8.3.1) (SEQ ID NO: 26) and Rma (EC 3.1.2.-) (SEQ ID NO: 27), or functional variants thereof having at least 70% homology, similarity or identity thereto,
      wherein Tle2 consists of Tle2 subunit A (EC 2.8.3.8) (SEQ ID NO: 19) and Tle2 subunit B (EC 2.8.3.9) (SEQ ID NO: 20), wherein Ghh2 consists of Ghh2 subunit A (SEQ ID NO: 22) and Ghh2 subunit B (SEQ ID NO: 23), and wherein Tme consists of Tme subunit A (SEQ ID NO: 24) and Tme subunit B (SEQ ID NO: 25), or functional variants thereof having at least 70% homology, similarity or identity thereto,
      and
   iii) an acetoacetate decarboxylase (EC 4.1.1.4), preferably Cac (SEQ ID NO: 28) or a functional variant thereof having at least 70% homology, similarity or identity thereto;
   iv) optionally an isopropanol dehydrogenase (EC 1.1.1.80), preferably Tbr (SEQ ID NO: 29) or a functional variant thereof having at least 70% homology, similarity or identity thereto;
b) cultivating the bacterial cell in a bioreactor comprising a cultivation broth at a temperature of between 42 and 80° C., such as between 50 and 75° C., for example at 60° C., thereby producing the one or more compounds;
c) recovering the one or more compounds produced during step b).

The thermophilic cell of the disclosure is capable of producing one or more volatile compounds, preferably acetone, butanone and/or isopropanol. The skilled person will know how to adapt the conditions under which the thermophilic cell is incubated in order to obtain one specific compounds. For example, the thermophilic cell is capable of producing acetone from acetyl-CoA, which the cell may be able to synthesise and/or which may be provided to the cells. In other cases, the thermophilic cell is capable of producing butanone from propionyl-CoA and acetyl-CoA, which the cell may be able to synthesise and/or which may be provided to the cells. If the cell expresses an isopropanol dehydrogenase, it can convert the produced acetone to isopropanol. The thermophilic cell is thus versatile: by changing the incubation conditions, all three compounds (acetone, butanone and isopropanol) can be obtained.

The thermophilic cell may be able to synthesise acetyl-CoA, for example when provided with acetic acid in the medium, or acetyl-CoA can be synthesized by the cell from other substrates or can be provided to the cell, e.g. if the cell has been engineered to be able to utilise extracellular acetyl-CoA, which could be provided in the fermentation broth. The thermophilic cell may be able to synthesise propionyl-CoA, for example when provided with propionic acid in the medium, or propionyl-CoA synthesized by the cell from other substrates or can be provided to the cell, e.g. if the cell has been engineered to be able to utilise extracellular propionyl-CoA, which could be provided in the fermentation broth.

In embodiments where the volatile compound to be produced is acetone, the cell is capable of converting acetyl-CoA to acetone via the following steps:
1) conversion of acetyl-CoA to acetoacetyl-CoA;
2) conversion of acetoacetyl-CoA to acetoacetate;
3) conversion of acetoacetate to acetone.

In embodiments where the volatile compound to be produced is butanone, the cell is capable of converting propionyl-CoA and acetyl-CoA to butanone via the following steps:
1) conversion of propionyl-CoA and acetyl-CoA to 3-ketovaleryl-CoA;
2) conversion of 3-ketovaleryl-CoA to 3-oxopentanoate;
3) conversion of 3-oxopentanoate to butanone.

In embodiments where the volatile compound to be produced is isopropanol, the cell is capable of producing acetone as described herein, and is further capable of converting acetone to isopropanol. This involves the following steps:
1) conversion of acetyl-CoA to acetoacetyl-CoA;
2) conversion of acetoacetyl-CoA to acetoacetate;
3) conversion of acetoacetate to acetone;
4) conversion of acetone to isopropanol.

Steps 1) to 3) above can be performed by the same enzymes independently of which volatile compound is to be produced. Production of isopropanol according to the present methods requires that the thermophilic cell expresses a further enzyme, which is not required for production of acetone or butanone, as detailed herein below.

Herein is thus provided a method of producing one or more compounds selected from acetone, butanone and isopropanol, said method comprising the steps of:
a) Providing a thermophilic cell, preferably a thermophilic bacterial or a thermophilic archaeal cell expressing:
   i) a first enzyme selected from:
an acetyl-CoA acetyltransferase (EC 2.3.1.9) selected from GHH_c20420 (SEQ ID NO: 1), Slip_0499 (SEQ ID NO: 2), Caur 1461 (SEQ ID NO: 3), Slip_0479 (SEQ ID NO: 4), Tfu_1520 (SEQ ID NO: 5), Tfu_0436 (SEQ ID NO: 6), Slip_0880 (SEQ ID NO: 7), Tfu_2394 (SEQ ID NO: 8), Slip_1236 (SEQ ID NO: 9), Caur_1540 (SEQ ID NO: 10), Tfu_0253 (SEQ ID NO: 11), CHY_1604 (SEQ ID NO: 14), CHY_1288 (SEQ ID NO: 15), Slip_2085 (SEQ ID NO: 16), Slip_0465 (SEQ ID NO: 17), Dde1 (SEQ ID NO: 59), Rxy2 (SEQ ID NO: 60) and CHY_1355 (SEQ ID NO: 18),
an enzyme of EC number 2.3.3.20 selected from the 3-oxoacyl-ACP synthase SVA_3859 (SEQ ID NO: 12) and the acyl-CoA:acyl-CoA alkyltransferase Despr_2661 (SEQ ID NO: 13); and
functional variants thereof having at least 70% homology, similarity or identity thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology, similarity or identity thereto;
   ii) a second enzyme selected from an acetate CoA transferase, a 3-oxoacid CoA transferase, an acyl CoA:acetate/3-ketoacid CoA-transferase, and an acyl-CoA thioesterase II,
wherein the second enzyme is selected from: Tle2, Dde2 (EC 2.8.3.5) (SEQ ID NO: 21), Ghh2 (EC 2.8.3.5), Tme (EC 2.8.3.8), Pth (EC 2.8.3.1) (SEQ ID NO: 26) and Rma (EC 3.1.2.-) (SEQ ID NO: 27), or functional variants thereof having at least 70% homology, similarity or identity thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology, similarity or identity thereto,
wherein Tle2 consists of Tle2 subunit A (EC 2.8.3.8) (SEQ ID NO: 19) and Tle2 subunit B (EC 2.8.3.9) (SEQ ID NO: 20), wherein Ghh2 consists of Ghh2 subunit A (SEQ ID NO: 22) and Ghh2 subunit B (SEQ ID NO: 23), and wherein Tme consists of Tme subunit A (SEQ ID NO: 24) and Tme subunit B (SEQ ID NO: 25), or functional variants having at least 70% homology, similarity or identity thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology, similarity or identity thereto,
and
   iii) an acetoacetate decarboxylase (EC 4.1.1.4), preferably Cac (SEQ ID NO: 28) or a functional variant thereof having at least 70% homology, similarity or identity thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology, similarity or identity thereto;
   iv) optionally an isopropanol dehydrogenase (EC 1.1.1.80), preferably Tbr (SEQ ID NO: 29) or a functional variant thereof having at least 70% homology, similarity or identity thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology, similarity or identity thereto;
b) cultivating the bacterial cell in a bioreactor comprising a cultivation broth at a temperature of between 42 and 80° C., such as between 50 and 75° C., for example at 60° C., thereby producing the one or more compounds;
c) recovering the one or more compounds produced during step b).

The thermophilic cell may be as described herein, in particular the cell may be a bacterial cell or an archaeal cell.

### First Enzyme

The first enzyme can be either an acetyl-CoA acetyltransferase, also termed thiolase, or a 3-oxoacyl-ACP synthase.

Thiolases catalyse either the conversion of:
i. two molecules of acetyl-CoA to acetoacetyl-CoA and coenzyme A (CoA), or
ii. one acetyl-CoA and one propionyl-CoA to 3-ketovaleryl-CoA and coenzyme A (CoA).

Which reaction actually occurs in the thermophilic cell will depend on which substrates are present in the broth, or on the metabolism of the particular cell used, as the skilled person well knows. If acetyl-CoA is present, then reaction i. will occur if both acetyl-CoA and propionyl-CoA are present, then reaction ii. or both reactions will occur. Supplementing the broth with acetic acid may increase the titer. Propionyl-CoA may be provided in the fermentation. The cell may also have been engineered to be capable of synthesising propionyl-CoA and/or acetyl-CoA, or to synthesise propionyl-CoA and/or acetyl-CoA in greater amounts than a corresponding non-engineered cell.

Reaction i. is required for production of acetone according to the present methods. Reaction ii. is required for the production of butanone according to the present methods.

In some embodiments the first enzyme is an acetyl-CoA acetyltransferase of EC number 2.3.1.9. These particular enzymes have a substrate preference for acetyl-CoA or propionyl-CoA and therefore preferably catalyse the reaction in the forward direction.

In some embodiments, the first enzyme is an enzyme of EC number 2.3.3.20, i.e. a 3-oxoacyl-ACP synthase (3-oxoacyl-[acyl-carrier-protein] synthase) or an acyl-CoA:acyl-CoA alkyltransferase. These catalyse the conversion of two molecules of acyl-CoA into one molecule of (2R)-2-alkyl-3-oxoalkanoate in the presence of water, thereby generating two molecules of coenzyme A (CoA).

The first enzyme is selected from the group consisting of GHH_c20420 (SEQ ID NO: 1), Slip_0499 (SEQ ID NO: 2), Caur_1461 (SEQ ID NO: 3), Slip_0479 (SEQ ID NO: 4), Tfu_1520 (SEQ ID NO: 5), Tfu_0436 (SEQ ID NO: 6), Slip_0880 (SEQ ID NO: 7), Tfu_2394 (SEQ ID NO: 8), Slip_1236 (SEQ ID NO: 9), Caur_1540 (SEQ ID NO: 10), Tfu_0253 (SEQ ID NO: 11), CHY_1604 (SEQ ID NO: 14), CHY_1288 (SEQ ID NO: 15), Slip_2085 (SEQ ID NO: 16), Slip_0465 (SEQ ID NO: 17), Dde1 (SEQ ID NO: 59), Rxy2 (SEQ ID NO: 60), CHY_1355 (SEQ ID NO: 18), SVA_3859 (SEQ ID NO: 12), Despr_2661 (SEQ ID NO: 13), and functional variants thereof having at least 70% homology, similarity or identity thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology, similarity or identity thereto. Preferably, the first enzyme is an acetyl-CoA acetyltransferase (EC 2.3.1.9) selected from GHH_c20420 as set forth in SEQ ID NO: 1, Slip 0499 as set forth in SEQ ID NO: 2, Caur 1461 as set forth in SEQ ID NO: 3, Slip_0479 as set forth in SEQ ID NO: 4, Slip_0880 as set forth in SEQ ID NO: 7, and Dde1 as set forth in SEQ ID NO: 59, and functional variants thereof having at least 70% identity, homology or similarity thereto.

In one embodiment, the first enzyme is GHH_c20420 (SEQ ID NO: 1), or a functional variant thereof having at least 70% homology, similarity or identity thereto. In another embodiment, the first enzyme is Slip_0499 (SEQ ID NO: 2), or a functional variant thereof having at least 70% homology, similarity or identity thereto. In another embodiment, the first enzyme is Caur 1461 (SEQ ID NO: 3), or a functional variant thereof having at least 70% homology, similarity or identity thereto. In another embodiment, the first enzyme is Slip_0479 (SEQ ID NO: 4), or a functional variant thereof having at least 70% homology, similarity or identity thereto. In another embodiment, the first enzyme is Tfu_1520 (SEQ ID NO: 5), or a functional variant thereof having at least 70% homology, similarity or identity thereto. In another embodiment, the first enzyme is Tfu_0436 (SEQ ID NO: 6), or a functional variant thereof having at least 70% homology, similarity or identity thereto. In another embodiment, the first enzyme is Slip_0880 (SEQ ID NO: 7), or a functional variant thereof having at least 70% homology, similarity or identity thereto. In another embodiment, the first enzyme is Tfu_2394 (SEQ ID NO: 8), or a functional variant thereof having at least 70% homology, similarity or identity thereto. In another embodiment, the first enzyme is Slip_1236 (SEQ ID NO: 9), or a functional variant thereof having at least 70% homology, similarity or identity thereto. In another embodiment, the first enzyme is Caur 1540 (SEQ ID NO: 10), or a functional variant thereof having at least 70% homology, similarity or identity thereto. In another embodiment, the first enzyme is Tfu_0253 (SEQ ID NO: 11), or a functional variant thereof having at least 70% homology, similarity or identity thereto. In another embodiment, the first enzyme is CHY_1604 (SEQ ID NO: 14), or a functional variant thereof having at least 70% homology, similarity or identity thereto. In another embodiment, the first enzyme is CHY_1288 (SEQ ID NO: 15), or a functional variant thereof having at least 70% homology, similarity or identity thereto. In another embodiment, the first enzyme is Slip_2085 (SEQ ID NO: 16), or a functional variant thereof having at least 70% homology, similarity or identity thereto. In another embodiment, the first enzyme is Slip_0465 (SEQ ID NO: 17), or a functional variant thereof having at least 70% homology, similarity or identity thereto. In another embodiment, the first enzyme is Dde1 (SEQ ID NO: 59) or a functional variant thereof having at least 70% homology, similarity or identity thereto. In another embodiment, the first enzyme is Rxy2 (SEQ ID NO: 60) or a functional variant thereof having at least 70% homology, similarity or identity thereto. In another embodiment, the first enzyme is CHY_1355 (SEQ ID NO: 18), or a functional variant thereof having at least 70% homology, similarity or identity thereto. In another embodiment, the first enzyme is SVA_3859 (SEQ ID NO: 12), or a functional variant thereof having at least 70% homology, similarity or identity thereto. In another embodiment, the first enzyme is Despr_2661 (SEQ ID NO: 13), or a functional variant thereof having at least 70% homology, similarity or identity thereto.

Functional variants of the above enzymes are modified versions of said enzyme which still retain at least some of the activity of the original enzyme. In the case of thermostable enzymes, the functional variant preferably also is thermostable. In some embodiments, the functional variant harbours mutations compared to the original enzyme, which preferably are not located within the active site of the enzyme. The skilled person knows how to determine if a variant of the first enzyme is functional or not. For example, potential thiolases can be incubated with acetoacetyl-CoA and CoA, and absorbance at 303 nm can be monitored. A decrease in the absorbance at 303 nm indicates that the potential thiolase can perform said reaction and has thiolase activity—it can thus be considered a functional variant. Potential 3-oxoacyl-ACP synthases can be incubated with acetyl-CoA (or acetyl-CoA and propionyl-CoA), with subsequent addition of 5,5′-dithio-bis-(2-nitrobenzoic acid), which reacts with a free thiol group of the released CoASH. The absorbance of the product can be monitored at 412 nm. Formation of the product indicates that the potential 3-oxoacyl-ACP synthase retains 3-oxoacyl-ACP synthase activity.

Enzymes of this type contain thiolase N-terminal domain (Pfam accession number PF00108), thiolase C-terminal domain (PF02803) and beta-ketoacyl synthase domain (PF00109), which contain the active center and participate in oligomerization of functional enzyme (Mathieu et al., 1997). Thus, functional variants of such enzymes preferably comprise said domains. Functional variants may have been engineered as is otherwise known in the art.

### Second Enzyme

The thermophilic cells employed in the present methods further express a second enzyme, which is selected from an acetate CoA transferase, a 3-oxoacid CoA transferase, acyl CoA:acetate/3-ketoacid CoA-transferase, and an acyl-CoA thioesterase II. This can be achieved by further engineering the cell.

Acetate CoA-transferases (EC 2.8.3.8) catalyse the conversion of acetate and an acyl-CoA to acetyl-CoA and a fatty acid. 3-oxoacid CoA-transferases (EC 2.8.3.5) catalyse the conversion of 3-oxoacyl-CoA and a succinate to 3-oxoacid and a 3-succinyl-CoA, or the conversion of 3-ketovaleryl-CoA+acetate to 3-oxopentanoate+acetyl-CoA. Acyl CoA:acetate/3-ketoacid CoA-transferases (EC 2.8.3.1) catalyse the conversion of acetyl-CoA and propanoate to acetate and propanoyl-CoA. Acyl-CoA thioesterases II (EC 3.1.2.-) catalyse the reaction of hydrolysis of acyl-CoA into fatty acid and CoASH.

Some of the above enzymes can catalyse different reactions. The type of reaction that actually occurs in the thermophilic cell will depend on which substrates are present in the broth or on how the cell has been engineered, as the skilled person well knows.

More specifically, the second enzyme is selected from: Tle2, Dde2 (EC 2.8.3.5) (SEQ ID NO: 21), Ghh2 (EC 2.8.3.5), Tme (EC 2.8.3.8), Pth (EC 2.8.3.1) (SEQ ID NO: 26), Rma (EC 3.1.2.-) (SEQ ID NO: 27), and functional variants thereof having at least 70% homology, similarity or identity thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology, similarity or identity thereto; wherein Tle2 consists of Tle2 subunit A (EC 2.8.3.8) (SEQ ID NO: 19) and Tle2 subunit B (EC 2.8.3.9) (SEQ ID NO: 20) or functional variants thereof having at least 70% homology, similarity or identity thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology, similarity or identity thereto; wherein Ghh2 consists of Ghh2 subunit A (SEQ ID NO: 22) and Ghh2 subunit B (SEQ ID NO: 23) or functional variants thereof having at least 70% homology, similarity or identity thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology, similarity or identity thereto; and wherein Tme consists of Tme subunit A (SEQ ID NO: 24) and Tme subunit B (SEQ ID NO: 25) or functional variants thereof having at least 70% homology, similarity or identity thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology, similarity or identity thereto.

Preferably, the second enzyme is Tle2 or Dde2 (SEQ ID NO: 21) or a functional variant thereof having at least 70% homology, similarity or identity thereto, wherein Tle2 consists of Tle2 subunit A (EC 2.8.3.8) (SEQ ID NO: 19) and Tle2 subunit B (EC 2.8.3.9) (SEQ ID NO: 20) or functional variants thereof having at least 70% homology, similarity or identity thereto.

In some embodiments, the second enzyme is Tle2 or a functional variant thereof having at least 70% homology, similarity or identity thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology, similarity or identity thereto. This enzyme consists of two subunits, subunit A as set forth in SEQ ID NO: 19, and subunit B as set forth in SEQ ID NO: 20. Subunit A has an EC number 2.8.3.8, and subunit B has an EC number 2.8.3.9. In some embodiments, the second enzyme is Tle2 which consists of Tle2 subunit A and Tle2 subunit B, as set forth in SEQ ID NO: 19 and SEQ ID NO: 20, respectively. In some embodiments, the second enzyme is Tle2 or a functional variant thereof having at least 70% homology, similarity or identity thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology, similarity or identity thereto. In some embodiments, the second enzyme is a functional variant of Tle2 consisting of Tle2 subunit A and a functional variant of Tle2 subunit B having at least 70% homology, similarity or identity to SEQ ID NO: 20, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology, similarity or identity thereto. In other embodiments, the second enzyme is a functional variant of Tle2 consisting of Tle2 subunit B and a functional variant of Tle2 subunit A having at least 70% homology, similarity or identity to SEQ ID NO: 19, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology, similarity or identity thereto. In some embodiments, the second enzyme is a functional variant of Tle2 consisting of a functional variant of Tle2 subunit A having at least 70% homology, similarity or identity to SEQ ID NO: 19, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology, similarity or identity thereto, and a functional variant of Tle2 subunit B having at least 70% homology, similarity or identity to SEQ ID NO: 20, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology, similarity or identity thereto.

In some embodiments, the second enzyme is an enzyme having an EC number 2.8.3.5. In some embodiments, the second enzyme is Dde2 (SEQ ID NO: 21) or a functional variant thereof having at least 70% homology, similarity or identity thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology, similarity or identity thereto.

In some embodiments, the second enzyme is Ghh2 or a functional variant thereof having at least 70% homology, similarity or identity thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology, similarity or identity thereto. This enzyme consists of two subunits, subunit A as set forth in SEQ ID NO: 22, and subunit B as set forth in SEQ ID NO: 23. Both subunits have EC number 2.8.3.5. In some embodiments, the second enzyme is Ghh2 which consists of Ghh2 subunit A and Ghh2 subunit B, as set forth in SEQ ID NO: 22 and SEQ ID NO: 23, respectively. In some embodiments, the second enzyme is Tle2 or a functional variant thereof having at least 70% homology, similarity or identity thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology, similarity or identity thereto. In some embodiments, the second enzyme is a functional variant of Ghh2 consisting of Ghh2 subunit A and a functional variant of Ghh2 subunit B having at least 70% homology, similarity or identity to SEQ ID NO: 23, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology, similarity or identity thereto. In other embodiments, the second enzyme is a functional variant of Ghh2 consisting of Ghh2 subunit B and a functional variant of Ghh2 subunit A having at least 70% homology, similarity or identity to SEQ ID NO: 22, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology, similarity or identity thereto. In some embodiments, the second enzyme is a functional variant of Ghh2 consisting of a functional variant of Ghh2 subunit A having at least 70% homology, similarity or identity to SEQ ID NO: 22, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology, similarity or identity thereto, and a functional variant of Ghh2 subunit B having at least 70% homology, similarity or identity to SEQ ID NO: 23, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology, similarity or identity thereto.

In some embodiments, the second enzyme is Tme or a functional variant thereof having at least 70% homology, similarity or identity thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology, similarity or identity thereto. This enzyme of EC number 2.8.3.8 consists of two subunits, subunit A as set forth in SEQ ID NO: 24, and subunit B as set forth in SEQ ID NO: 25. In some embodiments, the second enzyme is Tme which consists of Ghh2 subunit A and Tme subunit B, as set forth in SEQ ID NO: 24 and SEQ ID NO: 25, respectively. In some embodiments, the second enzyme is Tme or a functional variant thereof having at least 70% homology, similarity or identity thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology, similarity or identity thereto. In some embodiments, the second enzyme is a functional variant of Tme consisting of Tme subunit A and a functional variant of Tme subunit B having at least 70% homology, similarity or identity to SEQ ID NO: 25, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology, similarity or identity thereto. In other embodiments, the second enzyme is a functional variant of Tme consisting of Tme subunit B and a functional variant of Tme subunit A having at least 70% homology, similarity or identity to SEQ ID NO: 24, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology, similarity or identity thereto. In some embodiments, the second enzyme is a functional variant of Tme consisting of a functional variant of Tme subunit A having at least 70% homology, similarity or identity to SEQ ID NO: 24 and a functional variant of Tme subunit B having at least 70% homology, similarity or identity to SEQ ID NO: 25, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology, similarity or identity to SEQ ID NO: 24 and SEQ ID NO: 25, respectively.

In some embodiments, the second enzyme is an enzyme having an EC number 2.8.3.1. In some embodiments, the second enzyme is Pth (SEQ ID NO: 26) or a functional variant thereof having at least 70% homology, similarity or identity thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology, similarity or identity thereto.

In some embodiments, the second enzyme is an enzyme having an EC number 3.1.2.-. In some embodiments, the second enzyme is Rma (SEQ ID NO: 27) or a functional variant thereof having at least 70% homology, similarity or identity thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology, similarity or identity thereto.

Functional variants of the above enzymes are modified versions of said enzyme which still retain at least some of the activity of the original enzyme. In the case of thermostable enzymes, the functional variant preferably also is thermostable. In some embodiments, the functional variant harbours mutations compared to the original enzyme, which preferably are not located within the active site of the enzyme. The skilled person knows how to determine if a variant of the second enzyme is functional or not. For example, potential acetate coA-transferases of EC number 2.8.3.8 can be incubated with acetyl-CoA and lithium acetoacetate, and absorbance at 313 nm can be monitored to follow the formation of acetoacetyl-CoA. Potential 3-oxoacid CoA-transferases of EC number 2.8.3.5 can be tested as described herein above. Potential acyl CoA:acetate/3-ketoacid CoA-transferases (EC 2.8.3.1) can be tested as described herein above. Potential acyl-CoA thioesterases II (EC 3.1.2.-) can be tested as described herein above.

Enzymes of this type contain coenzyme A transferase domain (Pfam accession number PF01144) and acetyl-CoA hydrolase/transferase C-terminal domain (PF13336), which contain the active center and participate in oligomerization of functional enzyme. Thus, functional variants of such enzymes preferably comprise said domains. Functional variants may have been engineered as is otherwise known in the art.

### Acetoacetate Decarboxylase

The thermophilic cells employed in the present methods further express an acetoacetate decarboxylase. This enzyme has an EC number of 4.1.1.4, and catalyses the decarboxylation of acetoacetate, yielding acetone and carbon dioxide; the enzyme can also catalyse decarboxylation of said 3-oxopentanoate to butanone; it can also participate in the conversion of acetoacetyl-CoA to acetone or of 3-ketovaleryl-CoA to butanone. The expression of this enzyme in the thermophilic cells disclosed herein thus allows conversion of acetate to acetone, where the acetate is either provided to the cell, e.g. in the cultivation medium, or is produced by the cell. When the thermophilic cell is incubated in conditions where 3-oxopentanoate is produced, e.g. if the broth comprises propionic acid, the enzyme can catalyse the decarboxylation of said 3-oxopentanoate to butanone.

The acetoacetate decarboxylase is preferably a thermostable acetoacetate decarboxylase. In preferred embodiments, the acetoacetate decarboxylase is not native to a thermophilic microorganism, in particular the acetoacetate decarboxylase may be native to a Clostridium species such as Clostridium acetobutylicum. The acetoacetate decarboxylase Cac, as set forth in SEQ ID NO: 28, may be particularly advantageous for the present methods.

Thus in some embodiments, the acetoacetate decarboxylase is Cac as set forth in SEQ ID NO: 28, or a functional variant having at least 70% homology, similarity or identity thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology, similarity or identity thereto.

Functional variants of the above enzymes are modified versions of said enzymes which still retain at least some of the activity of the original enzymes. In the case of thermostable enzymes, the functional variant preferably also is thermostable. In some embodiments, the functional variant harbours mutations compared to the original enzyme, which preferably are not located within the active site of the enzyme. The skilled person knows how to determine if a variant of an acetoacetate decarboxylase is functional or not. For example, the potential acetoacetate decarboxylase can be incubated with lithium acetoacetate. The accompanying release of CO₂ which ensues can be monitored, e.g. manometrically. Release of CO₂ indicates that the tested enzyme has acetoacetate decarboxylase activity.

Enzymes of this type contain acetoacetate decarboxylase domain (Pfam accession number PF06314), which contain the active center and participate in oligomerization of functional enzyme. Amino acid residues Lys 115, Lys 116, Arg 29, Glu 61, Glu 76 in the active site are necessary for the activity of the enzyme (Ho et al., 2009). Thus, functional variants of such enzymes preferably comprise said domains and/or residues. Functional variants may have been engineered as is otherwise known in the art.

### Isopropanol Dehydrogenase

Also provided herein are cells and methods for the production of isopropanol in a thermophilic cell. For this, the thermophilic cell, in addition to the above enzymes, i.e. in addition to the first enzyme, the second enzyme and the acetoacetate decarboxylase, may further express an isopropanol dehydrogenase. This enzyme (EC 1.1.1.80) catalyzes the conversion of acetone to propan-2-ol. Thus a thermophilic cell capable of producing acetone as described herein can be further modified to express an isopropanol dehydrogenase which can then convert the produced acetone, or at least part thereof, to isopropanol.

In some embodiments, the isopropanol dehydrogenase is Tbr (SEQ ID NO: 29) or a functional variant thereof having at least 70% homology, similarity or identity thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology, similarity or identity thereto.

### Cultivation of the Thermophilic Cell

The thermophilic cell is cultivated in a reactor, for example a bioreactor or a fermenter, as is known in the art. In the context of the present disclosure, the cell is cultivated at “high” temperatures, i.e. temperatures above the conventional 37° C. normally employed for bacterial cultivations. The advantage of performing the cultivation at higher temperatures is that this allows facilitated recovery of the produced volatile compounds. Preferably, the thermophilic cell is cultivated at a temperature of between 42 and 80° C., such as between 50 and 75° C., for example at 60° C. In some embodiments, step b) is performed at a temperature of 42° C. or more, such as 43° C. or more, such as 44° C. or more, such as 45° C. or more, such as 46° C. or more, such as 47° C. or more, such as 48° C. or more, such as 49° C. or more, such as 50° C. or more, such as 51° C. or more, 52° C. or more, 53° C. or more, 54° C. or more, 55° C. or more, 56° C. or more, 57° C. or more, 58° C. or more, 59° C. or more, for example 60° C. or more. In some embodiments, the method is performed at a temperature of between 42 and 80° C., such as between 50 and 75° C., for example at 60° C. For example, the method is performed at a temperature of 42° C. or more, such as 43° C. or more, such as 44° C. or more, such as 45° C. or more, such as 46° C. or more, such as 47° C. or more, such as 48° C. or more, such as 49° C. or more, such as 50° C. or more, such as 51° C. or more, 52° C. or more, 53° C. or more, 54° C. or more, 55° C. or more, 56° C. or more, 57° C. or more, 58° C. or more, 59° C. or more, for example 60° C. or more.

The culture medium, or cultivation broth, comprises a fermentable carbon source as known in the art. In some embodiments, the medium comprises a substrate comprising carbohydrates. In particular, pentose or hexose sugars can be used as substrate, such as glucose, xylose, or a mixture thereof, or the medium may comprise or consist of a biomass hydrolysate, for example a lignocellulosic hydrolysate. In the present context the term “lignocellulosic hydrolysate” is intended to designate a lignocellulosic biomass which preferably has been subjected to a pre-treatment step whereby lignocellulosic material has been at least partially separated into cellulose, hemicellulose and lignin. The lignocellulosic material may typically be derived from plant material, such as straw, hay, garden refuse, comminuted wood, fruit hulls and seed hulls.

The pre-treatment method most often used is acid hydrolysis, where the lignocellulosic material is subjected to an acid such as sulphuric acid whereby the sugar polymers cellulose and hemicellulose are partly or completely hydrolysed to their constituent sugar monomers. Another type of lignocellulose hydrolysis is steam explosion, a process comprising heating of the lignocellulosic material by steam injection to a temperature of 190-230° C. A third method is wet oxidation wherein the material is treated with oxygen at 150-185° C. The pre-treatments can be followed by enzymatic hydrolysis to complete the release of sugar monomers. This pre-treatment step results in the hydrolysis of cellulose into glucose or cellobiose, while hemicellulose is transformed into the pentoses xylose and arabinose and the hexoses glucose, galactose and mannose. The pre-treatment step may in certain embodiments be supplemented with treatment resulting in further hydrolysis of the cellulose and hemicellulose. The purpose of such an additional hydrolysis treatment is to hydrolyse oligosaccharide and possibly polysaccharide species produced during the acid hydrolysis, wet oxidation, or steam explosion of cellulose and/or hemicellulose origin to form fermentable sugars (e.g. glucose, xylose and possibly other monosaccharides). Such further treatments may be either chemical or enzymatic. Chemical hydrolysis is typically achieved by treatment with an acid, such as treatment with aqueous sulphuric acid, at a temperature in the range of about 100-150° C. Enzymatic hydrolysis is typically performed by treatment with one or more appropriate enzymes such as cellulases, glucosidases and hemicellulases including xylanases.

Treatment of biomass to extract fermentable sugars can be carried out physically, chemically or biologically. Lignocellulose consists of cellulose, hemicellulose (a mixture of homo- and heteropolymers of xylose, arabinose, mannose, etc.), pectins and lignin organized in complex microstructures, which evolved to resist attacks by microorganisms and insects. Hence it can be relatively resistant to enzymatic decomposition and different methods of deconstruction are often combined. Pre-treatment is usually performed in order to make cellulose fibers more accessible to respective enzymes (cellulases), hydrolyze hemicelluloses and/or remove lignin. Typical process involves treatment with diluted acid or base at temperatures between 100° C. and 220° C. Due to its amorphous structure, hemicellulose is more readily hydrolyzed in this step, and up to 90% of its sugars can be recovered. However, this method also yields furfural and other products which may inhibit microorganisms' growth. Thus, enzyme cocktails collectively known as hemicellulases are sometimes used. On the other hand, cellulose is organized in microcrystalline fibers and is not easily hydrolyzed, but the degradation of cell wall matrix during pretreatment makes it more accessible for enzymes. Cellulases include: 1) endo-glucanases which act in the middle of cellulose molecule; 2) cellobiohydrolases which release cellobiose from the ends of cellulose; 3) β-D-glucosidases which hydrolyze cellobiose into glucose. As described above, enzymatic hydrolysis can be performed as a separate step (separate hydrolysis and fermentation, SHF) or simultaneously with fermentation (SSF). Recently, a complementary method has been proposed for complete solubilization of lignocellulose using biomass-derived γ-valerolactone.

Another attractive method is consolidated bioprocessing (CBP), which combines enzyme production, saccharification and fermentation in one step. This can be done by designing the producing cell of the present disclosure to express a heterologous metabolic pathway to degrade and utilize biomass.

Alternatively, the cell of the present disclosure may be cultivated together with another cell which is capable of degrading and utilising biomass, particularly at higher temperatures as described herein above. Using this setup, one microorganism, for example Clostridium thermocellum, degrades the biomass and provides the necessary substrates for the other microorganism, which can produce the volatile compounds as described above.

In some embodiments, the medium comprises glucose, xylose, or a mixture thereof. For example, the medium may comprise between 0.1% and between 20% (w/vol) glucose, xylose, or mixture thereof. For example, the medium comprises between 0.1% and 15% (w/vol) glucose, xylose, or mixture thereof, such as between 0.5% and 15% (w/vol) glucose, xylose, or mixture thereof, such as between 1% and 10% (w/vol) glucose, xylose, or mixture thereof, such as between 2% and 10% (w/vol) glucose, xylose, or mixture thereof, such as between 5% and 10% (w/vol) glucose, xylose, or mixture thereof, such as between 5% and 7.5% (w/vol) glucose, xylose, or mixture thereof. In some embodiments, the medium comprises at least 0.1% (w/vol) glucose, xylose, or mixture thereof, such as at least 0.25% (w/vol), such as at least 0.5% (w/vol), such as at least 0.75% (w/vol), such as at least 1% (w/vol), such as at least 2.5% (w/vol), such as at least 5% (w/vol), such as at least 10% (w/vol), such as at least 15% (w/vol), such as 20% (w/vol) glucose, xylose, or mixtures thereof.

In some embodiments, the thermophilic cell is an acetogenic thermophilic cell, in particular an acetogenic bacterial cell, which has been engineered to produce acetone, butanone or isopropanol. Such cells are capable of converting carbon monoxide, carbon dioxide, hydrogen, or a mixture thereof into acetyl-CoA, which is a substrate or a co-substrate for the above compounds. For example, acetogenic species include Moorella thermoacetica, Moorella thermoautotrophica and Thermoanaerobacter kivui.

In embodiments where production of acetone is desired, the culture medium may advantageously further comprise acetic acid or acetate. In some embodiments, the medium comprises between 0.05% and 5% (w/vol) acetic acid or acetate. For example, the medium comprises between 0.05% and 5% (w/vol) acetic acid or acetate or mixtures thereof, such as between 0.1% and 5% (w/vol), such as between 0.5% and 5% (w/vol), such as between 1% and 5% (w/vol), such as between 2% and 4% (w/vol) such as 3% acetic acid or acetate or mixtures thereof. In some embodiments, the medium comprises at least 0.05% (w/vol) acetic acid or acetate or mixtures thereof, such as at least 0.1% (w/vol), such as at least 0.5%, such as at least 1% (w/vol), such as at least 2% (w/vol), such as at least 3% (w/vol), such as at least 4% (w/vol), such as 5% (w/vol) acetic acid or acetate or mixtures thereof.

As described herein above, the cell may also have been engineered to synthesise acetyl-CoA more efficiently to be used as a substrate, or it may be cultivated with a microorganism which is capable of producing acetyl-CoA from the fermentable carbon source.

In embodiments where butanone production is desired, the culture medium may advantageously further comprise propionic acid or propionate. In some embodiments, the medium comprises between 0.05% and 2% (w/vol) propionic acid or propionate. For example, the medium comprises between 0.05% and 2% (w/vol) acetic acid or acetate or mixtures thereof, such as between 0.1% and 2% (w/vol), such as between 0.5% and 2% (w/vol), such as between 1% and 2% (w/vol) propionic acid, propionate or mixtures thereof. In some embodiments, the medium comprises at least 0.05% (w/vol) propionic acid, propionate or mixtures thereof thereof, such as at least 0.1% (w/vol), such as at least 0.5%, such as at least 1% (w/vol), such as 2% (w/vol) propionic acid, propionate or mixtures thereof.

As described herein above, the cell may also have been engineered to synthesise propionyl-CoA to be used as a substrate, or it may be cultivated with a microorganism which is capable of producing propionyl-CoA from the fermentable carbon source.

The present thermophilic cells can be cultivated in a continuous fermentation set-up, as is known in the art. This can be particularly advantageous as it also allows continuous product recovery, thereby preventing feedback inhibition and product toxicity. Because the cell is thermophilic and the cultivation is performed at higher temperatures than typically used for fermenting mesophilic cells, the volatile compounds will at least partly evaporate and can easily be recovered from the off-gas produced by the thermophilic cell. Thus in some embodiments, the methods disclosed herein further comprise recovering the one or more volatile compounds from the off-gas produced during the fermentation. In some embodiments, this is done by condensation.

In this setup an off gas is continuously removed from the bioreactor and cooled down to the temperature below the boiling point of the compound of interest. This leads to it going from the gas to liquid phase, at which point it is collected. Alternatively, the off gas can be flushed through the solvent, such as water, which has a temperature below the boiling point of the compound of interest. This process produces a saturated solution of this chemical. Alternatively, the off gas can be passed through a filter, such as for example activated charcoal, which binds the product.

Methods for Production of Acetone, Butanone and/or Isopropanol

The present methods preferably allow production of acetone with a titer of at least 0.8 g/L, such as at least 0.9 g/L, such as at least 1.0 g/L, such as at least 1.1 g/L, such as at least 1.2 g/L, such as at least 1.3 g/L, such as at least 1.4 g/L, such as at least 1.5 g/L, such as at least 1.6 g/L, such as at least 1.7 g/L, such as at least 1.8 g/L, such as at least 1.9 g/L, such as at least 2.0 g/L, such as at least 5 g/L, such as at least 7.5 g/L, such as at least 10 g/L, such as at least 12.5 g/L, such as at least 15 g/L, such as at least 20 g/L, such as at least 25 g/L, such as at least 50 g/L, such as at least 75 g/L, such as at least 100 g/L, such as at least 150 g/L, such as at least 250 g/L, or more.

In some embodiments, the method is for production of at least acetone, and the first enzyme is selected from the group consisting of CHY_1288 (SEQ ID NO: 15), CHY_1355 (SEQ ID NO: 18), Caur_1540 (SEQ ID NO: 10), GHH_c20420 (SEQ ID NO: 1), Caur 1461 (SEQ ID NO: 3) and Slip_0880 (SEQ ID NO: 7), or functional variants thereof having at least 70% homology, similarity or identity thereto, preferably Caur_1461 (SEQ ID NO: 3), Rxy2 (SEQ ID NO: 60), Slip_0880 (SEQ ID NO: 7) and Dde1 (SEQ ID NO: 59).

In some embodiments, the thermophilic cell is used for producing at least acetone, said cell expressing:
a) a first enzyme selected from CHY_1288 (SEQ ID NO: 15), CHY_1355 (SEQ ID NO: 18), Caur_1540 (SEQ ID NO: 10), GHH_c20420 (SEQ ID NO: 1), Caur_1461 (SEQ ID NO: 3), Dde1 (SEQ ID NO: 59), Rxy2 (SEQ ID NO: 60), and Slip_0880 (SEQ ID NO: 7), and functional variants thereof having at least 70% homology, similarity or identity thereto;
b) a second enzyme which is Tle2, wherein Tle2 consists of Tle2 subunit A (EC 2.8.3.8) (SEQ ID NO: 19) and Tle2 subunit B (EC 2.8.3.9) (SEQ ID NO: 20), or functional variants thereof having at least 70% homology, similarity or identity thereto, and
c) the acetoacetate decarboxylase Cac (SEQ ID NO: 28) or a functional variant thereof having at least 70% homology, similarity or identity thereto.

In one embodiment, the thermophilic cell expresses: CHY_1288 (SEQ ID NO: 15), Tle2 and Cac, or functional variants thereof having at least 70% homology, similarity or identity thereto, wherein Tle2 consists of Tle2 subunit A (EC 2.8.3.8) (SEQ ID NO: 19) and Tle2 subunit B (EC 2.8.3.9) (SEQ ID NO: 20), or functional variants thereof having at least 70% homology, similarity or identity thereto.

In another embodiment, the thermophilic cell expresses: Caur_1540 (SEQ ID NO: 10), Tle2 and Cac, or functional variants thereof having at least 70% homology, similarity or identity thereto, wherein Tle2 consists of Tle2 subunit A (EC 2.8.3.8) (SEQ ID NO: 19) and Tle2 subunit B (EC 2.8.3.9) (SEQ ID NO: 20), or functional variants thereof having at least 70% homology, similarity or identity thereto.

In another embodiment, the thermophilic cell expresses: GHH_c20420 (SEQ ID NO: 1), Tle2 and Cac, or functional variants thereof having at least 70% homology, similarity or identity thereto, wherein Tle2 consists of Tle2 subunit A (EC 2.8.3.8) (SEQ ID NO: 19) and Tle2 subunit B (EC 2.8.3.9) (SEQ ID NO: 20), or functional variants thereof having at least 70% homology, similarity or identity thereto.

In another embodiment, the thermophilic cell expresses: Caur_1461 (SEQ ID NO: 3), Tle2 and Cac, or functional variants thereof having at least 70% homology, similarity or identity thereto, wherein Tle2 consists of Tle2 subunit A (EC 2.8.3.8) (SEQ ID NO: 19) and Tle2 subunit B (EC 2.8.3.9) (SEQ ID NO: 20), or functional variants thereof having at least 70% homology, similarity or identity thereto.

In another embodiment, the thermophilic cell expresses: Slip_0880 (SEQ ID NO: 7), Tle2 and Cac, or functional variants thereof having at least 70% homology, similarity or identity thereto, wherein Tle2 consists of Tle2 subunit A (EC 2.8.3.8) (SEQ ID NO: 19) and Tle2 subunit B (EC 2.8.3.9) (SEQ ID NO: 20), or functional variants thereof having at least 70% homology, similarity or identity thereto.

In another embodiment, the thermophilic cell expresses: Dde1 (SEQ ID NO: 59), Tle2 and Cac, or functional variants thereof having at least 70% homology, similarity or identity thereto, wherein Tle2 consists of Tle2 subunit A (EC 2.8.3.8) (SEQ ID NO: 19) and Tle2 subunit B (EC 2.8.3.9) (SEQ ID NO: 20), or functional variants thereof having at least 70% homology, similarity or identity thereto.

In another embodiment, the thermophilic cell expresses: Rxy2 (SEQ ID NO: 60)), Tle2 and Cac, or functional variants thereof having at least 70% homology, similarity or identity thereto, wherein Tle2 consists of Tle2 subunit A (EC 2.8.3.8) (SEQ ID NO: 19) and Tle2 subunit B (EC 2.8.3.9) (SEQ ID NO: 20), or functional variants thereof having at least 70% homology, similarity or identity thereto.

In some embodiments, the thermophilic cell is used for producing at least acetone, said cell expressing:
a) a first enzyme selected from CHY_1288 (SEQ ID NO: 15), CHY_1355 (SEQ ID NO: 18), Caur_1540 (SEQ ID NO: 10), GHH_c20420 (SEQ ID NO: 1), Caur_1461 (SEQ ID NO: 3), Rxy2 (SEQ ID NO: 60), Slip_0880 (SEQ ID NO: 7) Dde1 (SEQ ID NO: 59) and functional variants thereof having at least 70% homology, similarity or identity thereto;
b) a second enzyme which is Dde2 (EC 2.8.3.5) (SEQ ID NO: 21), or a functional variant thereof having at least 70% homology, similarity or identity thereto, and
c) the acetoacetate decarboxylase Cac (SEQ ID NO: 28) or a functional variant thereof having at least 70% homology, similarity or identity thereto.

In one embodiment, the thermophilic cell expresses: CHY_1288 (SEQ ID NO: 15), Dde2 (SEQ ID NO: 21) and Cac (SEQ ID NO: 28), or functional variants thereof having at least 70% homology, similarity or identity thereto.

In another embodiment, the thermophilic cell expresses: Caur_1540 (SEQ ID NO: 10), Dde2 (SEQ ID NO: 21) and Cac (SEQ ID NO: 28), or functional variants thereof having at least 70% homology, similarity or identity thereto.

In another embodiment, the thermophilic cell expresses: GHH_c20420 (SEQ ID NO: 1), Dde2 (SEQ ID NO: 21) and Cac (SEQ ID NO: 28), or functional variants thereof having at least 70% homology, similarity or identity thereto.

In another embodiment, the thermophilic cell expresses: Caur_1461 (SEQ ID NO: 3), Dde2 (SEQ ID NO: 21) and Cac (SEQ ID NO: 28), or functional variants thereof having at least 70% homology, similarity or identity thereto.

In another embodiment, the thermophilic cell expresses: Slip_0880 (SEQ ID NO: 7), Dde2 (SEQ ID NO: 21) and Cac (SEQ ID NO: 28), or functional variants thereof having at least 70% homology, similarity or identity thereto.

In another embodiment, the thermophilic cell expresses: Dde1 (SEQ ID NO: 59), Dde2 (SEQ ID NO: 21) and Cac (SEQ ID NO: 28), or functional variants thereof having at least 70% homology, similarity or identity thereto.

In another embodiment, the thermophilic cell expresses: Rxy2 (SEQ ID NO: 60), Dde2 (SEQ ID NO: 21) and Cac (SEQ ID NO: 28), or functional variants thereof having at least 70% homology, similarity or identity thereto.

In some embodiments, the thermophilic cell is used for producing at least acetone, said cell expressing:
a) a first enzyme selected from CHY_1288 (SEQ ID NO: 15), CHY_1355 (SEQ ID NO: 18), Caur_1540 (SEQ ID NO: 10), GHH_c20420 (SEQ ID NO: 1), Caur_1461 (SEQ ID NO: 3), Rxy2 (SEQ ID NO: 60), Slip_0880 (SEQ ID NO: 7) and Dde1 (SEQ ID NO: 59), and functional variants thereof having at least 70% homology, similarity or identity thereto;
b) a second enzyme which is Ghh2 (EC 2.8.3.5), wherein Ghh2 consists of Ghh2 subunit A (SEQ ID NO: 22) and Ghh2 subunit B (SEQ ID NO: 23), or functional variants thereof having at least 70% homology, similarity or identity thereto, and
c) the acetoacetate decarboxylase Cac (SEQ ID NO: 28) or a functional variant thereof having at least 70% homology, similarity or identity thereto.

In one embodiment, the thermophilic cell expresses: CHY 1288 (SEQ ID NO: 15), Ghh2 and Cac (SEQ ID NO: 28), or functional variants thereof having at least 70% homology, similarity or identity thereto, wherein Ghh2 consists of Ghh2 subunit A (SEQ ID NO: 22) and Ghh2 subunit B (SEQ ID NO: 23), or functional variants thereof having at least 70% homology, similarity or identity thereto.

In another embodiment, the thermophilic cell expresses: Caur_1540 (SEQ ID NO: 10), Ghh2 and Cac (SEQ ID NO: 28), or functional variants thereof having at least 70% homology, similarity or identity thereto, wherein Ghh2 consists of Ghh2 subunit A (SEQ ID NO: 22) and Ghh2 subunit B (SEQ ID NO: 23), or functional variants thereof having at least 70% homology, similarity or identity thereto.

In another embodiment, the thermophilic cell expresses: GHH_c20420 (SEQ ID NO: 1), Ghh2 and Cac (SEQ ID NO: 28), or functional variants thereof having at least 70% homology, similarity or identity thereto, wherein Ghh2 consists of Ghh2 subunit A (SEQ ID NO: 22) and Ghh2 subunit B (SEQ ID NO: 23), or functional variants thereof having at least 70% homology, similarity or identity thereto.

In another embodiment, the thermophilic cell expresses: Caur_1461 (SEQ ID NO: 3), Ghh2 and Cac (SEQ ID NO: 28), or functional variants thereof having at least 70% homology, similarity or identity thereto, wherein Ghh2 consists of Ghh2 subunit A (SEQ ID NO: 22) and Ghh2 subunit B (SEQ ID NO: 23), or functional variants thereof having at least 70% homology, similarity or identity thereto.

In another embodiment, the thermophilic cell expresses: Slip_0880 (SEQ ID NO: 7), Ghh2 and Cac (SEQ ID NO: 28), or functional variants thereof having at least 70% homology, similarity or identity thereto, wherein Ghh2 consists of Ghh2 subunit A (SEQ ID NO: 22) and Ghh2 subunit B (SEQ ID NO: 23), or functional variants thereof having at least 70% homology, similarity or identity thereto.

In another embodiment, the thermophilic cell expresses: Dde1 (SEQ ID NO: 59), Ghh2 and Cac (SEQ ID NO: 28), or functional variants thereof having at least 70% homology, similarity or identity thereto, wherein Ghh2 consists of Ghh2 subunit A (SEQ ID NO: 22) and Ghh2 subunit B (SEQ ID NO: 23), or functional variants thereof having at least 70% homology, similarity or identity thereto.

In another embodiment, the thermophilic cell expresses: Dde1 (SEQ ID NO: 59), Ghh2 and Cac (SEQ ID NO: 28), or functional variants thereof having at least 70% homology, similarity or identity thereto, wherein Ghh2 consists of Ghh2 subunit A (SEQ ID NO: 22) and Ghh2 subunit B (SEQ ID NO: 23), or functional variants thereof having at least 70% homology, similarity or identity thereto.

In another embodiment, the thermophilic cell expresses: Rxy2 (SEQ ID NO: 60), Ghh2 and Cac (SEQ ID NO: 28), or functional variants thereof having at least 70% homology, similarity or identity thereto, wherein Ghh2 consists of Ghh2 subunit A (SEQ ID NO: 22) and Ghh2 subunit B (SEQ ID NO: 23), or functional variants thereof having at least 70% homology, similarity or identity thereto.

In another embodiment, the thermophilic cell expresses: Rxy2 (SEQ ID NO: 60), Ghh2 and Cac (SEQ ID NO: 28), or functional variants thereof having at least 70% homology, similarity or identity thereto, wherein Ghh2 consists of Ghh2 subunit A (SEQ ID NO: 22) and Ghh2 subunit B (SEQ ID NO: 23), or functional variants thereof having at least 70% homology, similarity or identity thereto.

In some embodiments, the thermophilic cell is used for producing at least acetone, said cell expressing:
a) a first enzyme selected from CHY_1288 (SEQ ID NO: 15), CHY_1355 (SEQ ID NO: 18), Caur_1540 (SEQ ID NO: 10), GHH_c20420 (SEQ ID NO: 1), Caur_1461 (SEQ ID NO: 3), Rxy2 (SEQ ID NO: 60), Slip 0880 (SEQ ID NO: 7) and Dde1 (SEQ ID NO: 59), and functional variants thereof having at least 70% homology, similarity or identity thereto;
b) a second enzyme which is Tme (EC 2.8.3.8), wherein Tme consists of Tme subunit A (SEQ ID NO: 24) and Tme subunit B (SEQ ID NO: 25), or functional variants thereof having at least 70% homology, similarity or identity thereto, and
c) the acetoacetate decarboxylase Cac (SEQ ID NO: 28) or a functional variant thereof having at least 70% homology, similarity or identity thereto.

In one embodiment, the thermophilic cell expresses: CHY_1288 (SEQ ID NO: 15), Tme and Cac (SEQ ID NO: 28), or functional variants thereof having at least 70% homology, similarity or identity thereto, wherein Tme consists of Tme subunit A (SEQ ID NO: 24) and Tme subunit B (SEQ ID NO: 25), or functional variants thereof having at least 70% homology, similarity or identity thereto.

In another embodiment, the thermophilic cell expresses: Caur_1540 (SEQ ID NO: 10), Tme and Cac (SEQ ID NO: 28), or functional variants thereof having at least 70% homology, similarity or identity thereto, wherein Tme consists of Tme subunit A (SEQ ID NO: 24) and Tme subunit B (SEQ ID NO: 25), or functional variants thereof having at least 70% homology, similarity or identity thereto.

In another embodiment, the thermophilic cell expresses: GHH_c20420 (SEQ ID NO: 1), Tme and Cac (SEQ ID NO: 28), or functional variants thereof having at least 70% homology, similarity or identity thereto, wherein Tme consists of Tme subunit A (SEQ ID NO: 24) and Tme subunit B (SEQ ID NO: 25), or functional variants thereof having at least 70% homology, similarity or identity thereto.

In another embodiment, the thermophilic cell expresses: Caur_1461 (SEQ ID NO: 3), Tme and Cac (SEQ ID NO: 28), or functional variants thereof having at least 70% homology, similarity or identity thereto, wherein Tme consists of Tme subunit A (SEQ ID NO: 24) and Tme subunit B (SEQ ID NO: 25), or functional variants thereof having at least 70% homology, similarity or identity thereto.

In another embodiment, the thermophilic cell expresses: Slip_0880 (SEQ ID NO: 7), Tme and Cac (SEQ ID NO: 28), or functional variants thereof having at least 70% homology, similarity or identity thereto, wherein Tme consists of Tme subunit A (SEQ ID NO: 24) and Tme subunit B (SEQ ID NO: 25), or functional variants thereof having at least 70% homology, similarity or identity thereto.

In another embodiment, the thermophilic cell expresses: Dde1 (SEQ ID NO: 59), Tme and Cac (SEQ ID NO: 28), or functional variants thereof having at least 70% homology, similarity or identity thereto, wherein Tme consists of Tme subunit A (SEQ ID NO: 24) and Tme subunit B (SEQ ID NO: 25), or functional variants thereof having at least 70% homology, similarity or identity thereto.

In another embodiment, the thermophilic cell expresses: Rxy2 (SEQ ID NO: 60), Tme and Cac (SEQ ID NO: 28), or functional variants thereof having at least 70% homology, similarity or identity thereto, wherein Tme consists of Tme subunit A (SEQ ID NO: 24) and Tme subunit B (SEQ ID NO: 25), or functional variants thereof having at least 70% homology, similarity or identity thereto.

In some embodiments, the thermophilic cell is used for producing at least acetone, said cell expressing:
a) a first enzyme selected from CHY_1288 (SEQ ID NO: 15), CHY_1355 (SEQ ID NO: 18), Caur_1540 (SEQ ID NO: 10), GHH_c20420 (SEQ ID NO: 1), Caur_1461 (SEQ ID NO: 3), Dde1 (SEQ ID NO: 59), Rxy2 (SEQ ID NO: 60), and Slip_0880 (SEQ ID NO: 7), and functional variants thereof having at least 70% homology, similarity or identity thereto;
b) a second enzyme which is Pth (EC 2.8.3.1) (SEQ ID NO: 26), or a functional variant thereof having at least 70% homology, similarity or identity thereto, and
c) the acetoacetate decarboxylase Cac (SEQ ID NO: 28) or a functional variant thereof having at least 70% homology, similarity or identity thereto.

In one embodiment, the thermophilic cell expresses: CHY_1288 (SEQ ID NO: 15), Pth (SEQ ID NO: 26) and Cac (SEQ ID NO: 28), or functional variants thereof having at least 70% homology, similarity or identity thereto.

In another embodiment, the thermophilic cell expresses: Caur_1540 (SEQ ID NO: 10), Pth (SEQ ID NO: 26) and Cac (SEQ ID NO: 28), or functional variants thereof having at least 70% homology, similarity or identity thereto.

In another embodiment, the thermophilic cell expresses: GHH_c20420 (SEQ ID NO: 1), Pth (SEQ ID NO: 26) and Cac (SEQ ID NO: 28), or functional variants thereof having at least 70% homology, similarity or identity thereto.

In another embodiment, the thermophilic cell expresses: Caur_1461 (SEQ ID NO: 3), Pth (SEQ ID NO: 26) and Cac (SEQ ID NO: 28), or functional variants thereof having at least 70% homology, similarity or identity thereto.

In another embodiment, the thermophilic cell expresses: Slip_0880 (SEQ ID NO: 7), Pth (SEQ ID NO: 26) and Cac (SEQ ID NO: 28), or functional variants thereof having at least 70% homology, similarity or identity thereto.

In another embodiment, the thermophilic cell expresses: Dde1 (SEQ ID NO: 59), Pth (SEQ ID NO: 26) and Cac (SEQ ID NO: 28), or functional variants thereof having at least 70% homology, similarity or identity thereto.

In another embodiment, the thermophilic cell expresses: Rxy2 (SEQ ID NO: 60), Pth (SEQ ID NO: 26) and Cac (SEQ ID NO: 28), or functional variants thereof having at least 70% homology, similarity or identity thereto.

In some embodiments, the thermophilic cell is used for producing at least acetone, said cell expressing:
a) a first enzyme selected from CHY_1288 (SEQ ID NO: 15), CHY_1355 (SEQ ID NO: 18), Caur_1540 (SEQ ID NO: 10), GHH_c20420 (SEQ ID NO: 1), Caur_1461 (SEQ ID NO: 3), Dde1 (SEQ ID NO: 59), Rxy2 (SEQ ID NO: 60) and Slip_0880 (SEQ ID NO: 7), and functional variants thereof having at least 70% homology, similarity or identity thereto;
b) a second enzyme which is Rma (EC 3.1.2.-) (SEQ ID NO: 27), or a functional variant thereof having at least 70% homology, similarity or identity thereto, and
c) the acetoacetate decarboxylase Cac (SEQ ID NO: 28) or a functional variant thereof having at least 70% homology, similarity or identity thereto.

In one embodiment, the thermophilic cell expresses: CHY_1288 (SEQ ID NO: 15), Rma (SEQ ID NO: 27) and Cac (SEQ ID NO: 28), or functional variants thereof having at least 70% homology, similarity or identity thereto.

In another embodiment, the thermophilic cell expresses: Caur_1540 (SEQ ID NO: 10), Rma (SEQ ID NO: 27) and Cac (SEQ ID NO: 28), or functional variants thereof having at least 70% homology, similarity or identity thereto.

In another embodiment, the thermophilic cell expresses: GHH_c20420 (SEQ ID NO: 1), Rma (SEQ ID NO: 27) and Cac (SEQ ID NO: 28), or functional variants thereof having at least 70% homology, similarity or identity thereto.

In another embodiment, the thermophilic cell expresses: Caur_1461 (SEQ ID NO: 3), Rma (SEQ ID NO: 27) and Cac (SEQ ID NO: 28), or functional variants thereof having at least 70% homology, similarity or identity thereto.

In another embodiment, the thermophilic cell expresses: Slip_0880 (SEQ ID NO: 7), Rma (SEQ ID NO: 27) and Cac (SEQ ID NO: 28), or functional variants thereof having at least 70% homology, similarity or identity thereto.

In another embodiment, the thermophilic cell expresses: Dde1 (SEQ ID NO: 59), Rma (SEQ ID NO: 27) and Cac (SEQ ID NO: 28), or functional variants thereof having at least 70% homology, similarity or identity thereto.

In another embodiment, the thermophilic cell expresses: Rxy2 (SEQ ID NO: 60), Rma (SEQ ID NO: 27) and Cac (SEQ ID NO: 28), or functional variants thereof having at least 70% homology, similarity or identity thereto.

Preferably, the thermophilic cell can produce at least acetone, and expresses Cac as set forth in SEQ ID NO: 28 or a functional variant thereof having at least 70% identity or similarity thereto and one of the following combinations of first and second enzymes:
i) Dde1 as set forth in SEQ ID NO: 59 and Dde2 as set forth in SEQ ID NO: 21; or functional variants thereof having at least 70% identity or similarity thereto; or
ii) Caur_1461 as set forth in SEQ ID NO: 3 and Tle2; or functional variants thereof having at least 70% identity or similarity thereto; or
iii) Slip_0880 as set forth in SEQ ID NO: 7 and Tle2; or functional variants thereof having at least 70% identity or similarity thereto;
   wherein Tle2 consists of Tle2 subunit A (EC 2.8.3.8) as set forth in SEQ ID NO: 19 and Tle2 subunit B (EC 2.8.3.9) as set forth in SEQ ID NO: 20 or functional variants thereof having at least 70% identity or similarity thereto.

The present methods allow production of butanone with a titer of at least 0.05 g/L, such as at least 0.075 g/L, such as at least 0.1 g/L, such as at least 0.2 g/L, such as at least 0.3 g/L, such as at least 0.4 g/L, such as at least 0.5 g/L, such as at least 0.75 g/L, such as at least 1.0 g/L, such as at least 2.0 g/L, such as at least 3.0 g/L, such as at least 4.0 g/L, such as at least 5.0 g/L, such as at least 7.5 g/L, such as at least 10.0 g/L, such as at least 25 g/L, such as at least 50 g/L, such as at least 75 g/L, such as at least 100 g/L, such as at least 150 g/L, such as at least 250 g/L, or more.

In some embodiments, the method is for production of at least butanone, and the first enzyme is selected from the group consisting of GHH_c20420 (SEQ ID NO: 1), Slip_0499 (SEQ ID NO: 2), Caur_1461 (SEQ ID NO: 3), Dde1 (SEQ ID NO: 59), Rxy2 (SEQ ID NO: 60), Slip_0479 (SEQ ID NO: 4), Tfu_1520 (SEQ ID NO: 5), and Tfu_0436 (SEQ ID NO: 6), or functional variants thereof having at least 70% homology, similarity or identity thereto, preferably the first enzyme is GHH_c20420 (SEQ ID NO: 1), Slip_0499 (SEQ ID NO: 2), Caur_1461 (SEQ ID NO: 3) or Slip_0479 (SEQ ID NO: 4).

In some embodiments, the thermophilic cell is used for producing at least butanone, said cell expressing:
a) a first enzyme selected from GHH_c20420 (SEQ ID NO: 1), Slip_0499 (SEQ ID NO: 2), Caur_1461 (SEQ ID NO: 3), Dde1 (SEQ ID NO: 59), Rxy2 (SEQ ID NO: 60), Slip_0479 (SEQ ID NO: 4), Tfu_1520 (SEQ ID NO: 5), and Tfu_0436 (SEQ ID NO: 6), and functional variants thereof having at least 70% homology, similarity or identity thereto;
b) a second enzyme which is Tle2, wherein Tle2 consists of Tle2 subunit A (EC 2.8.3.8) (SEQ ID NO: 19) and Tle2 subunit B (EC 2.8.3.9) (SEQ ID NO: 20), or functional variants thereof having at least 70% homology, similarity or identity thereto, and
c) the acetoacetate decarboxylase Cac (SEQ ID NO: 28) or a functional variant thereof having at least 70% homology, similarity or identity thereto.

In one embodiment, the thermophilic cell expresses: GHH_c20420 (SEQ ID NO: 1), Tle2 and Cac (SEQ ID NO: 28), or functional variants thereof having at least 70% homology, similarity or identity thereto, wherein Tle2 consists of Tle2 subunit A (EC 2.8.3.8) (SEQ ID NO: 19) and Tle2 subunit B (EC 2.8.3.9) (SEQ ID NO: 20), or functional variants thereof having at least 70% homology, similarity or identity thereto.

In another embodiment, the thermophilic cell expresses: Slip_0499 (SEQ ID NO: 2) Tle2 and Cac (SEQ ID NO: 28), or functional variants thereof having at least 70% homology, similarity or identity thereto, wherein Tle2 consists of Tle2 subunit A (EC 2.8.3.8) (SEQ ID NO: 19) and Tle2 subunit B (EC 2.8.3.9) (SEQ ID NO: 20), or functional variants thereof having at least 70% homology, similarity or identity thereto.

In another embodiment, the thermophilic cell expresses: Caur_1461 (SEQ ID NO: 3), Tle2 and Cac (SEQ ID NO: 28), or functional variants thereof having at least 70% homology, similarity or identity thereto, wherein Tle2 consists of Tle2 subunit A (EC 2.8.3.8) (SEQ ID NO: 19) and Tle2 subunit B (EC 2.8.3.9) (SEQ ID NO: 20), or functional variants thereof having at least 70% homology, similarity or identity thereto.

In another embodiment, the thermophilic cell expresses: Slip_0479 (SEQ ID NO: 4), Tle2 and Cac (SEQ ID NO: 28), or functional variants thereof having at least 70% homology, similarity or identity thereto, wherein Tle2 consists of Tle2 subunit A (EC 2.8.3.8) (SEQ ID NO: 19) and Tle2 subunit B (EC 2.8.3.9) (SEQ ID NO: 20), or functional variants thereof having at least 70% homology, similarity or identity thereto.

In another embodiment, the thermophilic cell expresses: Dde1 (SEQ ID NO: 59), Tle2 and Cac (SEQ ID NO: 28), or functional variants thereof having at least 70% homology, similarity or identity thereto, wherein Tle2 consists of Tle2 subunit A (EC 2.8.3.8) (SEQ ID NO: 19) and Tle2 subunit B (EC 2.8.3.9) (SEQ ID NO: 20), or functional variants thereof having at least 70% homology, similarity or identity thereto.

In another embodiment, the thermophilic cell expresses: Rxy2 (SEQ ID NO: 60), Tle2 and Cac (SEQ ID NO: 28), or functional variants thereof having at least 70% homology, similarity or identity thereto, wherein Tle2 consists of Tle2 subunit A (EC 2.8.3.8) (SEQ ID NO: 19) and Tle2 subunit B (EC 2.8.3.9) (SEQ ID NO: 20), or functional variants thereof having at least 70% homology, similarity or identity thereto.

In another embodiment, the thermophilic cell expresses: Tfu_1520 (SEQ ID NO: 5), Tle2 and Cac (SEQ ID NO: 28), or functional variants thereof having at least 70% homology, similarity or identity thereto, wherein Tle2 consists of Tle2 subunit A (EC 2.8.3.8) (SEQ ID NO: 19) and Tle2 subunit B (EC 2.8.3.9) (SEQ ID NO: 20), or functional variants thereof having at least 70% homology, similarity or identity thereto.

In another embodiment, the thermophilic cell expresses: Tfu_0436 (SEQ ID NO: 6), Tle2 and Cac (SEQ ID NO: 28), or functional variants thereof having at least 70% homology, similarity or identity thereto, wherein Tle2 consists of Tle2 subunit A (EC 2.8.3.8) (SEQ ID NO: 19) and Tle2 subunit B (EC 2.8.3.9) (SEQ ID NO: 20), or functional variants thereof having at least 70% homology, similarity or identity thereto.

In some embodiments, the thermophilic cell is used for producing at least butanone, said cell expressing:
a) a first enzyme selected from GHH_c20420 (SEQ ID NO: 1), Slip_0499 (SEQ ID NO: 2), Caur_1461 (SEQ ID NO: 3), Dde1 (SEQ ID NO: 59), Rxy2 (SEQ ID NO: 60), Slip_0479 (SEQ ID NO: 4), Tfu_1520 (SEQ ID NO: 5), or Tfu_0436 (SEQ ID NO: 6), and functional variants thereof having at least 70% homology, similarity or identity thereto;
b) a second enzyme which is Dde2 (EC 2.8.3.5) (SEQ ID NO: 21), or a functional variant thereof having at least 70% homology, similarity or identity thereto, and
c) the acetoacetate decarboxylase Cac (SEQ ID NO: 28) or a functional variant thereof having at least 70% homology, similarity or identity thereto.

In one embodiment, the thermophilic cell expresses: GHH_c20420 (SEQ ID NO: 1), Dde2 (SEQ ID NO: 21) and Cac (SEQ ID NO: 28), or functional variants thereof having at least 70% homology, similarity or identity thereto.

In another embodiment, the thermophilic cell expresses: Slip_0499 (SEQ ID NO: 2), Dde2 (SEQ ID NO: 21) and Cac (SEQ ID NO: 28), or functional variants thereof having at least 70% homology, similarity or identity thereto.

In another embodiment, the thermophilic cell expresses: Caur_1461 (SEQ ID NO: 3), Dde2 (SEQ ID NO: 21) and Cac (SEQ ID NO: 28), or functional variants thereof having at least 70% homology, similarity or identity thereto.

In another embodiment, the thermophilic cell expresses: Slip_0479 (SEQ ID NO: 4), Dde2 (SEQ ID NO: 21) and Cac (SEQ ID NO: 28), or functional variants thereof having at least 70% homology, similarity or identity thereto.

In another embodiment, the thermophilic cell expresses: Dde1 (SEQ ID NO: 59), Dde2 (SEQ ID NO: 21) and Cac (SEQ ID NO: 28), or functional variants thereof having at least 70% homology, similarity or identity thereto.

In another embodiment, the thermophilic cell expresses: Rxy2 (SEQ ID NO: 60), Dde2 (SEQ ID NO: 21) and Cac (SEQ ID NO: 28), or functional variants thereof having at least 70% homology, similarity or identity thereto.

In another embodiment, the thermophilic cell expresses: Tfu_1520 (SEQ ID NO: 5), Dde2 (SEQ ID NO: 21) and Cac (SEQ ID NO: 28), or functional variants thereof having at least 70% homology, similarity or identity thereto.

In another embodiment, the thermophilic cell expresses: Tfu_0436 (SEQ ID NO: 6), Dde2 (SEQ ID NO: 21) and Cac (SEQ ID NO: 28), or functional variants thereof having at least 70% homology, similarity or identity thereto.

In some embodiments, the thermophilic cell is used for producing at least butanone, said cell expressing:
a) a first enzyme selected from GHH_c20420 (SEQ ID NO: 1), Slip_0499 (SEQ ID NO: 2), Caur_1461 (SEQ ID NO: 3), Dde1 (SEQ ID NO: 59), Rxy2 (SEQ ID NO: 60), Slip_0479 (SEQ ID NO: 4), Tfu_1520 (SEQ ID NO: 5), and Tfu_0436 (SEQ ID NO: 6), and functional variants thereof having at least 70% homology, similarity or identity thereto;
b) a second enzyme which is Ghh2 (EC 2.8.3.5), wherein Ghh2 consists of Ghh2 subunit A (SEQ ID NO: 22) and Ghh2 subunit B (SEQ ID NO: 23), or functional variants thereof having at least 70% homology, similarity or identity thereto, and
c) the acetoacetate decarboxylase Cac (SEQ ID NO: 21) or a functional variant thereof having at least 70% homology, similarity or identity thereto.

In one embodiment, the thermophilic cell expresses: GHH_c20420 (SEQ ID NO: 1), Ghh2 and Cac (SEQ ID NO: 21), or functional variants thereof having at least 70% homology, similarity or identity thereto, wherein Ghh2 consists of Ghh2 subunit A (SEQ ID NO: 22) and Ghh2 subunit B (SEQ ID NO: 23), or functional variants thereof having at least 70% homology, similarity or identity thereto.

In another embodiment, the thermophilic cell expresses: Slip_0499 (SEQ ID NO: 2), Ghh2 and Cac (SEQ ID NO: 21), or functional variants thereof having at least 70% homology, similarity or identity thereto, wherein Ghh2 consists of Ghh2 subunit A (SEQ ID NO: 22) and Ghh2 subunit B (SEQ ID NO: 23), or functional variants thereof having at least 70% homology, similarity or identity thereto.

In another embodiment, the thermophilic cell expresses: Caur_1461 (SEQ ID NO: 3), Ghh2 and Cac (SEQ ID NO: 21), or functional variants thereof having at least 70% homology, similarity or identity thereto, wherein Ghh2 consists of Ghh2 subunit A (SEQ ID NO: 22) and Ghh2 subunit B (SEQ ID NO: 23), or functional variants thereof having at least 70% homology, similarity or identity thereto.

In another embodiment, the thermophilic cell expresses: Slip_0479 (SEQ ID NO: 4), Ghh2 and Cac (SEQ ID NO: 21), or functional variants thereof having at least 70% homology, similarity or identity thereto, wherein Ghh2 consists of Ghh2 subunit A (SEQ ID NO: 22) and Ghh2 subunit B (SEQ ID NO: 23), or functional variants thereof having at least 70% homology, similarity or identity thereto.

In another embodiment, the thermophilic cell expresses: Dde1 (SEQ ID NO: 59), Ghh2 and Cac (SEQ ID NO: 21), or functional variants thereof having at least 70% homology, similarity or identity thereto, wherein Ghh2 consists of Ghh2 subunit A (SEQ ID NO: 22) and Ghh2 subunit B (SEQ ID NO: 23), or functional variants thereof having at least 70% homology, similarity or identity thereto.

In another embodiment, the thermophilic cell expresses: Rxy2 (SEQ ID NO: 60), Ghh2 and Cac (SEQ ID NO: 21), or functional variants thereof having at least 70% homology, similarity or identity thereto, wherein Ghh2 consists of Ghh2 subunit A (SEQ ID NO: 22) and Ghh2 subunit B (SEQ ID NO: 23), or functional variants thereof having at least 70% homology, similarity or identity thereto.

In another embodiment, the thermophilic cell expresses: Tfu_1520 (SEQ ID NO: 5), Ghh2 and Cac (SEQ ID NO: 21), or functional variants thereof having at least 70% homology, similarity or identity thereto, wherein Ghh2 consists of Ghh2 subunit A (SEQ ID NO: 22) and Ghh2 subunit B (SEQ ID NO: 23), or functional variants thereof having at least 70% homology, similarity or identity thereto.

In another embodiment, the thermophilic cell expresses: Tfu_0436 (SEQ ID NO: 6), Ghh2 and Cac (SEQ ID NO: 21), or functional variants thereof having at least 70% homology, similarity or identity thereto, wherein Ghh2 consists of Ghh2 subunit A (SEQ ID NO: 22) and Ghh2 subunit B (SEQ ID NO: 23), or functional variants thereof having at least 70% homology, similarity or identity thereto.

In some embodiments, the thermophilic cell is used for producing at least butanone, said cell expressing:
a) a first enzyme selected from GHH_c20420 (SEQ ID NO: 1), Slip_0499 (SEQ ID NO: 2), Caur_1461 (SEQ ID NO: 3), Dde1 (SEQ ID NO: 59), Rxy2 (SEQ ID NO: 60), Slip_0479 (SEQ ID NO: 4), Tfu_1520 (SEQ ID NO: 5), and Tfu_0436 (SEQ ID NO: 6), and functional variants thereof having at least 70% homology, similarity or identity thereto;
b) a second enzyme which is Tme (EC 2.8.3.8), wherein Tme consists of Tme subunit A (SEQ ID NO: 24) and Tme subunit B (SEQ ID NO: 25), or functional variants thereof having at least 70% homology, similarity or identity thereto, and
c) the acetoacetate decarboxylase Cac (SEQ ID NO: 28) or a functional variant thereof having at least 70% homology, similarity or identity thereto.

In one embodiment, the thermophilic cell expresses: GHH_c20420 (SEQ ID NO: 1), Tme and Cac (SEQ ID NO: 21), or functional variants thereof having at least 70% homology, similarity or identity thereto, wherein Tme consists of Tme subunit A (SEQ ID NO: 24) and Tme subunit B (SEQ ID NO: 25), or functional variants thereof having at least 70% homology, similarity or identity thereto.

In another embodiment, the thermophilic cell expresses: Slip_0499 (SEQ ID NO: 2), Tme and Cac (SEQ ID NO: 21), or functional variants thereof having at least 70% homology, similarity or identity thereto, wherein Tme consists of Tme subunit A (SEQ ID NO: 24) and Tme subunit B (SEQ ID NO: 25), or functional variants thereof having at least 70% homology, similarity or identity thereto.

In another embodiment, the thermophilic cell expresses: Caur_1461 (SEQ ID NO: 3), Tme and Cac (SEQ ID NO: 21), or functional variants thereof having at least 70% homology, similarity or identity thereto, wherein Tme consists of Tme subunit A (SEQ ID NO: 24) and Tme subunit B (SEQ ID NO: 25), or functional variants thereof having at least 70% homology, similarity or identity thereto.

In another embodiment, the thermophilic cell expresses: Slip_0479 (SEQ ID NO: 4), Tme and Cac (SEQ ID NO: 21), or functional variants thereof having at least 70% homology, similarity or identity thereto, wherein Tme consists of Tme subunit A (SEQ ID NO: 24) and Tme subunit B (SEQ ID NO: 25), or functional variants thereof having at least 70% homology, similarity or identity thereto.

In another embodiment, the thermophilic cell expresses: Dde1 (SEQ ID NO: 59), Tme and Cac (SEQ ID NO: 21), or functional variants thereof having at least 70% homology, similarity or identity thereto, wherein Tme consists of Tme subunit A (SEQ ID NO: 24) and Tme subunit B (SEQ ID NO: 25), or functional variants thereof having at least 70% homology, similarity or identity thereto.

In another embodiment, the thermophilic cell expresses: Rxy2 (SEQ ID NO: 60), Tme and Cac (SEQ ID NO: 21), or functional variants thereof having at least 70% homology, similarity or identity thereto, wherein Tme consists of Tme subunit A (SEQ ID NO: 24) and Tme subunit B (SEQ ID NO: 25), or functional variants thereof having at least 70% homology, similarity or identity thereto.

In another embodiment, the thermophilic cell expresses: Tfu_1520 (SEQ ID NO: 5), Tme and Cac (SEQ ID NO: 21), or functional variants thereof having at least 70% homology, similarity or identity thereto, wherein Tme consists of Tme subunit A (SEQ ID NO: 24) and Tme subunit B (SEQ ID NO: 25), or functional variants thereof having at least 70% homology, similarity or identity thereto.

In another embodiment, the thermophilic cell expresses: Tfu_0436 (SEQ ID NO: 6), Tme and Cac (SEQ ID NO: 21), or functional variants thereof having at least 70% homology, similarity or identity thereto, wherein Tme consists of Tme subunit A (SEQ ID NO: 24) and Tme subunit B (SEQ ID NO: 25), or functional variants thereof having at least 70% homology, similarity or identity thereto.

In some embodiments, the thermophilic cell is used for producing at least butanone, said cell expressing:
a) a first enzyme selected from GHH_c20420 (SEQ ID NO: 1), Slip_0499 (SEQ ID NO: 2), Caur_1461 (SEQ ID NO: 3), Dde1 (SEQ ID NO: 59), Rxy2 (SEQ ID NO: 60), Slip_0479 (SEQ ID NO: 4), Tfu_1520 (SEQ ID NO: 5), and Tfu_0436 (SEQ ID NO: 6), and functional variants thereof having at least 70% homology, similarity or identity thereto;
b) a second enzyme which is Pth (EC 2.8.3.1) (SEQ ID NO: 26), or a functional variant thereof having at least 70% homology, similarity or identity thereto, and
c) the acetoacetate decarboxylase Cac (SEQ ID NO: 28) or a functional variant thereof having at least 70% homology, similarity or identity thereto.

In one embodiment, the thermophilic cell expresses: GHH_c20420 (SEQ ID NO: 1), Pth (SEQ ID NO: 26) and Cac (SEQ ID NO: 28), or functional variants thereof having at least 70% homology, similarity or identity thereto.

In another embodiment, the thermophilic cell expresses: Slip_0499 (SEQ ID NO: 2), Pth (SEQ ID NO: 26) and Cac (SEQ ID NO: 28), or functional variants thereof having at least 70% homology, similarity or identity thereto.

In another embodiment, the thermophilic cell expresses: Caur_1461 (SEQ ID NO: 3), Pth (SEQ ID NO: 26) and Cac (SEQ ID NO: 28), or functional variants thereof having at least 70% homology, similarity or identity thereto.

In another embodiment, the thermophilic cell expresses: Slip_0479 (SEQ ID NO: 4), Pth (SEQ ID NO: 26) and Cac (SEQ ID NO: 28), or functional variants thereof having at least 70% homology, similarity or identity thereto.

In another embodiment, the thermophilic cell expresses: Dde1 (SEQ ID NO: 59), Pth (SEQ ID NO: 26) and Cac (SEQ ID NO: 28), or functional variants thereof having at least 70% homology, similarity or identity thereto.

In another embodiment, the thermophilic cell expresses: Rxy2 (SEQ ID NO: 60), Pth (SEQ ID NO: 26) and Cac (SEQ ID NO: 28), or functional variants thereof having at least 70% homology, similarity or identity thereto.

In another embodiment, the thermophilic cell expresses: Tfu_1520 (SEQ ID NO: 5), Pth (SEQ ID NO: 26) and Cac (SEQ ID NO: 28), or functional variants thereof having at least 70% homology, similarity or identity thereto.

In another embodiment, the thermophilic cell expresses: Tfu_0436 (SEQ ID NO: 6), Pth (SEQ ID NO: 26) and Cac (SEQ ID NO: 28), or functional variants thereof having at least 70% homology, similarity or identity thereto.

In some embodiments, the thermophilic cell is used for producing at least butanone, said cell expressing:
a) a first enzyme selected from GHH_c20420 (SEQ ID NO: 1), Slip_0499 (SEQ ID NO: 2), Caur_1461 (SEQ ID NO: 3), Dde1 (SEQ ID NO: 59), Rxy2 (SEQ ID NO: 60), Slip_0479 (SEQ ID NO: 4), Tfu_1520 (SEQ ID NO: 5), and Tfu_0436 (SEQ ID NO: 6), and functional variants thereof having at least 70% homology, similarity or identity thereto;
b) a second enzyme which is Rma (EC 3.1.2.-) (SEQ ID NO: 27), or a functional variant thereof having at least 70% homology, similarity or identity thereto, and
c) the acetoacetate decarboxylase Cac (SEQ ID NO: 28) or a functional variant thereof having at least 70% homology, similarity or identity thereto.

In one embodiment, the thermophilic cell expresses: GHH_c20420 (SEQ ID NO: 1), Rma (SEQ ID NO: 27) and Cac (SEQ ID NO: 28), or functional variants thereof having at least 70% homology, similarity or identity thereto.

In another embodiment, the thermophilic cell expresses: Slip_0499 (SEQ ID NO: 2), Rma (SEQ ID NO: 27) and Cac (SEQ ID NO: 28), or functional variants thereof having at least 70% homology, similarity or identity thereto.

In another embodiment, the thermophilic cell expresses: Caur_1461 (SEQ ID NO: 3), Rma (SEQ ID NO: 27) and Cac (SEQ ID NO: 28), or functional variants thereof having at least 70% homology, similarity or identity thereto.

In another embodiment, the thermophilic cell expresses: Slip_0479 (SEQ ID NO: 4), Rma (SEQ ID NO: 27) and Cac (SEQ ID NO: 28), or functional variants thereof having at least 70% homology, similarity or identity thereto.

In another embodiment, the thermophilic cell expresses: Dde1 (SEQ ID NO: 59), Rma (SEQ ID NO: 27) and Cac (SEQ ID NO: 28), or functional variants thereof having at least 70% homology, similarity or identity thereto.

In another embodiment, the thermophilic cell expresses: Rxy2 (SEQ ID NO: 60), Rma (SEQ ID NO: 27) and Cac (SEQ ID NO: 28), or functional variants thereof having at least 70% homology, similarity or identity thereto.

In another embodiment, the thermophilic cell expresses: Tfu_1520 (SEQ ID NO: 5), Rma (SEQ ID NO: 27) and Cac (SEQ ID NO: 28), or functional variants thereof having at least 70% homology, similarity or identity thereto.

In another embodiment, the thermophilic cell expresses: Tfu_0436 (SEQ ID NO: 6), Rma (SEQ ID NO: 27) and Cac (SEQ ID NO: 28), or functional variants thereof having at least 70% homology, similarity or identity thereto.

Preferably, the thermophilic cell can produce at least butanone, and expresses Cac as set forth in SEQ ID NO: 28 or a functional variant thereof having at least 70% identity or similarity thereto and one of the following combinations of first and second enzymes:
i) Caur_1461 as set forth in SEQ ID NO: 3 and Tle2; or functional variants thereof having at least 70% identity or similarity thereto; or
ii) GHH_c20420 as set forth in SEQ ID NO: 1 and Tle2; or functional variants thereof having at least 70% identity or similarity thereto; or
iii) Slip_0499 as set forth in SEQ ID NO: 2 and Tle2; or functional variants thereof having at least 70% identity or similarity thereto; or
iv) Slip_0479 as set forth in SEQ ID NO: 4 and Tle2; or functional variants thereof having at least 70% identity or similarity thereto;
   wherein Tle2 consists of Tle2 subunit A (EC 2.8.3.8) as set forth in SEQ ID NO: 19 and Tle2 subunit B (EC 2.8.3.9) as set forth in SEQ ID NO: 20 or functional variants thereof having at least 70% identity or similarity thereto.

The above thermophilic cells can produce acetone from acetyl-CoA, for example in the presence of a suitable substrate such as acetic acid, which may be synthesised by the cell, and/or butanone from propionyl-CoA, for example in the presence of a suitable substrate such as propionic acid. Supplementing the broth with acetic acid may increase the titer. Propionyl-CoA may be provided in the fermentation. The cell may also have been engineered to be capable of synthesising propionyl-CoA and/or acetyl-CoA, or to synthesise propionyl-CoA and/or acetyl-CoA in greater amounts than a corresponding non-engineered cell. Any of the above thermophilic cells may in addition to the above express an isopropanol dehydrogenase, in particular Tbr (SEQ ID NO: 29) or a functional variant thereof having at least 70% homology, similarity or identity thereto. This allows at least part of the acetone produced by the thermophilic cell (or provided to the cell) to be converted to isopropanol.

In some embodiments, at least isopropanol is produced. The isopropanol titer is preferably at least 0.05 g/L, such as at least 0.075 g/L, such as at least 0.1 g/L, such as at least 0.2 g/L, such as at least 0.3 g/L, such as at least 0.4 g/L, such as at least 0.5 g/L, such as at least 0.75 g/L, such as at least 1.0 g/L, such as at least 2.0 g/L, such as at least 3.0 g/L, such as at least 4.0 g/L, such as at least 5.0 g/L, such as at least 7.5 g/L, such as at least 10.0 g/L, such as at least 12.5 g/L, such as at least 15 g/L, such as at least 20 g/L, such as at least 25 g/L, such as at least 50 g/L, such as at least 75 g/L, such as at least 100 g/L, such as at least 150 g/L, such as at least 250 g/L, or more.

Preferably, the thermophilic cell can produce at least acetone and isopropanol, and expresses Cac as set forth in SEQ ID NO: 28 and Tbr (SEQ ID NO: 29) or functional variants thereof having at least 70% identity or similarity thereto and one of the following combinations of first and second enzymes:
i) Dde1 as set forth in SEQ ID NO: 59 and Dde2 as set forth in SEQ ID NO: 21; or functional variants thereof having at least 70% identity or similarity thereto; or
ii) Caur_1461 as set forth in SEQ ID NO: 3 and Tle2; or functional variants thereof having at least 70% identity or similarity thereto; or
iii) Slip_0880 as set forth in SEQ ID NO: 7 and Tle2; or functional variants thereof having at least 70% identity or similarity thereto;
   wherein Tle2 consists of Tle2 subunit A (EC 2.8.3.8) as set forth in SEQ ID NO: 19 and Tle2 subunit B (EC 2.8.3.9) as set forth in SEQ ID NO: 20 or functional variants thereof having at least 70% identity or similarity thereto.

In some embodiments, the thermophilic cell can produce at least butanone and isopropanol, and expresses Cac as set forth in SEQ ID NO: 28 and Tbr (SEQ ID NO: 29) or functional variants thereof having at least 70% identity or similarity thereto and one of the following combinations of first and second enzymes:
i) Caur_1461 as set forth in SEQ ID NO: 3 and Tle2; or functional variants thereof having at least 70% identity or similarity thereto; or
ii) GHH_c20420 as set forth in SEQ ID NO: 1 and Tle2; or functional variants thereof having at least 70% identity or similarity thereto; or
iii) Slip_0499 as set forth in SEQ ID NO: 2 and Tle2; or functional variants thereof having at least 70% identity or similarity thereto; or
iv) Slip_0479 as set forth in SEQ ID NO: 4 and Tle2; or functional variants thereof having at least 70% identity or similarity thereto;
   wherein Tle2 consists of Tle2 subunit A (EC 2.8.3.8) as set forth in SEQ ID NO: 19 and Tle2 subunit B (EC 2.8.3.9) as set forth in SEQ ID NO: 20 or functional variants thereof having at least 70% identity or similarity thereto.

### Thermophilic Cells and Nucleic Acid Constructs

Useful thermophilic cells have been described in detail herein above. Once the skilled person has determined which enzymes to express in the thermophilic cells of the present disclosure, he/she will have no difficulty to do so.

The enzymes may be expressed by introducing nucleic acid sequences encoding each of them in the cell, for example on a plasmid, or by genomic integration. For example, genes can be inserted into a replicating plasmid, which is then transformed into the cell by means of electroporation. A gene in the same plasmid, which encodes an antibiotic resistance marker, will ensure that only the transformed cells survive in the medium with the respective antibiotic, but other selection systems may also be utilized. Genomic integration is achieved, for example, by using a plasmid, for example a temperature sensitive plasmid, carrying the genes of interest. In the right conditions under non-permissible temperatures, the plasmid will undergo double crossover by homologous recombination, leaving a seamless marker-free integration into the genomic DNA. Gene expression can be controlled as is known in the art, for example by using appropriate vectors, plasmids, promoters or codon-optimisation. For example, in embodiments where the thermophilic cell is a Geobacillus cell, in particular a Geobacillus thermoglucosiadus cell, the method described in Pogrebnyakov et al., 2017, may be employed.

Accordingly, the thermophilic cells disclosed herein may comprise one or more polynucleotides encoding the first enzyme, the second enzyme and the acetoacetate decarboxylase as described herein above, and optionally encoding the isopropanol dehydrogenase as described herein above. Each of said polynucleotides may encode a single enzyme, or it may encode several enzymes which then get expressed simultaneously.

Herein is thus also provided a nucleic acid construct for modifying a thermophilic cell selected from a thermophilic bacterial cell and a thermophilic archaeal cell, comprising:
i) a polynucleotide encoding an acetyl-CoA acetyltransferase (EC 2.3.1.9) or a functional variant thereof having at least 70% homology, similarity or identity thereto, wherein the acetyl-CoA acetyltransferase is selected from GHH_c20420 (SEQ ID NO: 1), Slip_0499 (SEQ ID NO: 2), Caur_1461 (SEQ ID NO: 3), Slip_0479 (SEQ ID NO: 4), Tfu_1520 (SEQ ID NO: 5), Tfu_0436 (SEQ ID NO: 6), Slip_0880 (SEQ ID NO: 7), Tfu_2394 (SEQ ID NO: 8), Slip_1236 (SEQ ID NO: 9), Caur_1540 (SEQ ID NO: 10), Tfu_0253 (SEQ ID NO: 11), CHY_1604 (SEQ ID NO: 14), CHY_1288 (SEQ ID NO: 15), Slip_2085 (SEQ ID NO: 16), Slip_0465 (SEQ ID NO: 17), Dde1 (SEQ ID NO: 59), Rxy2 (SEQ ID NO: 60) and CHY_1355 (SEQ ID NO: 18), and/or an enzyme of EC number 2.3.3.20 selected from the 3-oxoacyl-ACP synthase SVA_3859 (SEQ ID NO: 12) and the acyl-CoA:acyl-CoA alkyltransferase Despr_2661 (SEQ ID NO: 13);
ii) a polynucleotide encoding a second enzyme selected from an acetate CoA transferase, a 3-oxoacid CoA transferase, an acyl CoA:acetate/3-ketoacid CoA-transferase, and an acyl-CoA thioesterase II
   wherein the second enzyme is selected from: Tle2, Dde2 (EC 2.8.3.5) (SEQ ID NO: 21), Ghh2 (EC 2.8.3.5), Tme (EC 2.8.3.8), Pth (EC 2.8.3.1) (SEQ ID NO: 26) and Rma (EC 3.1.2.-) (SEQ ID NO: 27), or functional variants thereof having at least 70% homology, similarity or identity thereto,
   wherein Tle2 consists of Tle2 subunit A (EC 2.8.3.8) (SEQ ID NO: 19) and Tle2 subunit B (EC 2.8.3.9) (SEQ ID NO: 20), wherein Ghh2 consists of Ghh2 subunit A (SEQ ID NO: 22) and Ghh2 subunit B (SEQ ID NO: 23), and wherein Tme consists of Tme subunit A (SEQ ID NO: 24) and Tme subunit B (SEQ ID NO: 25), or functional variants thereof having at least 70% homology, similarity or identity thereto, and
iii) a polynucleotide encoding an acetoacetate decarboxylase (EC 4.1.1.4) or a functional variant thereof having at least 70% homology, similarity or identity thereto, preferably Cac (SEQ ID NO: 28).

In particular, herein is provided a nucleic acid construct for modifying a thermophilic cell selected from a thermophilic bacterial cell and a thermophilic archaeal cell, comprising:
i) a polynucleotide encoding an acetyl-CoA acetyltransferase (EC 2.3.1.9) or a functional variant thereof having at least 70% identity or similarity thereto having acetyl-CoA acetyltransferase activity, wherein the acetyl-CoA acetyltransferase is selected from GHH_c20420 as set forth in SEQ ID NO: 1, Slip_0499 as set forth in SEQ ID NO: 2, Caur_1461 as set forth in SEQ ID NO: 3, Slip_0479 as set forth in SEQ ID NO: 4, Slip_0880 as set forth in SEQ ID NO: 7, and Dde1 as set forth in SEQ ID NO: 59;
ii) a polynucleotide encoding a second enzyme selected from an acetate CoA transferase, a 3-oxoacid CoA transferase, an acyl CoA:acetate/3-ketoacid CoA-transferase, and an acyl-CoA thioesterase II
   wherein the second enzyme is selected from: Tle2 and Dde2 (EC 2.8.3.5) as set forth in SEQ ID NO: 21, or functional variants thereof having at least 70% identity or similarity thereto having at least 70% identity or similarity thereto having acetate CoA transferase, 3-oxoacid CoA transferase, acyl CoA:acetate/3-ketoacid CoA-transferase, or acyl-CoA thioesterase II activity,
   wherein Tle2 consists of Tle2 subunit A (EC 2.8.3.8) as set forth in SEQ ID NO: 19 and Tle2 subunit B (EC 2.8.3.9) as set forth in SEQ ID NO: 20, or functional variants thereof having at least 70% identity or similarity thereto having at least 70% identity or similarity thereto having acetate CoA transferase, 3-oxoacid CoA transferase, acyl CoA:acetate/3-ketoacid CoA-transferase, or acyl-CoA thioesterase II activity, and
iii) a polynucleotide encoding an acetoacetate decarboxylase (EC 4.1.1.4) or a functional variant thereof having at least 70% identity or similarity thereto having acetoacetate decarboxylase activity, wherein the acetoacetate decarboxylase is Cac as set forth in SEQ ID NO: 28, and
iv) optionally a polynucleotide encoding an isopropanol dehydrogenase (EC 1.1.1.80), wherein the isopropanol dehydrogenase is Tbr as set forth in SEQ ID NO: 29 or a functional variant thereof having at least 70% identity or similarity thereto having isopropanol dehydrogenase activity.

Expression of each polynucleotide may be under the control of an inducible promoter, or under the control of a constitutive promoter.

Thus also disclosed herein is a nucleic acid construct for modifying a thermophilic cell, in particular a thermophilic bacterial cell or a thermophilic archaeal cell, which can be used to construct the thermophilic cells of the present disclosure, i.e. cells capable of producing acetone, butanone and/or isopropanol.

Said nucleic acid construct comprises or consists of:
i) a polynucleotide encoding an acetyl-CoA acetyltransferase (EC 2.3.1.9) or a functional variant thereof having at least 70% homology, similarity or identity thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology, similarity or identity thereto, wherein the acetyl-CoA acetyltransferase is selected from GHH_c20420 (SEQ ID NO: 1), Slip_0499 (SEQ ID NO: 2), Caur_1461 (SEQ ID NO: 3), Slip_0479 (SEQ ID NO: 4), Tfu_1520 (SEQ ID NO: 5), Tfu_0436 (SEQ ID NO: 6), Slip_0880 (SEQ ID NO: 7), Tfu_2394 (SEQ ID NO: 8), Slip_1236 (SEQ ID NO: 9), Caur_1540 (SEQ ID NO: 10), Tfu_0253 (SEQ ID NO: 11), CHY_1604 (SEQ ID NO: 14), CHY_1288 (SEQ ID NO: 15), Slip_2085 (SEQ ID NO: 16), Slip_0465 (SEQ ID NO: 17), Dde1 (SEQ ID NO: 59), Rxy2 (SEQ ID NO: 60) and CHY 1355 (SEQ ID NO: 18), and/or an enzyme of EC number 2.3.3.20 selected from the 3-oxoacyl-ACP synthase SVA_3859 (SEQ ID NO: 12) and the acyl-CoA:acyl-CoA alkyltransferase Despr_2661 (SEQ ID NO: 13);
ii) a polynucleotide encoding a second enzyme selected from an acetate CoA transferase, a 3-oxoacid CoA transferase, an acyl CoA:acetate/3-ketoacid CoA-transferase, and an acyl-CoA thioesterase II
   wherein the second enzyme is selected from: Tle2, Dde2 (EC 2.8.3.5) (SEQ ID NO: 21), Ghh2 (EC 2.8.3.5), Tme (EC 2.8.3.8), Pth (EC 2.8.3.1) (SEQ ID NO: 26) and Rma (EC 3.1.2.-) (SEQ ID NO: 27), or functional variants thereof having at least 70% homology, similarity or identity thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology, similarity or identity thereto,
   wherein Tle2 consists of Tle2 subunit A (EC 2.8.3.8) (SEQ ID NO: 19) and Tle2 subunit B (EC 2.8.3.9) (SEQ ID NO: 20), wherein Ghh2 consists of Ghh2 subunit A (SEQ ID NO: 22) and Ghh2 subunit B (SEQ ID NO: 23), and wherein Tme consists of Tme subunit A (SEQ ID NO: 24) and Tme subunit B (SEQ ID NO: 25), or functional variants having at least 70% homology, similarity or identity thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology, similarity or identity thereto, and
iii) a polynucleotide encoding an acetoacetate decarboxylase (EC 4.1.1.4) or a functional variant thereof having at least 70% homology, similarity or identity thereto, preferably Cac (SEQ ID NO: 28), such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology, similarity or identity thereto.

The nucleic acid construct may comprise or consist of one or more polynucleotides. It will be understood that the term “nucleic acid constructs” may refer to one nucleic acid molecule, or to a plurality of nucleic acid molecules, comprising the relevant nucleic acid sequences. The nucleic acid construct may thus be one nucleic acid molecule, which may encode several enzymes, or it may be several nucleic acid molecules, each comprising one sequence encoding an enzyme. The relevant nucleic acid sequences may thus be comprised on one vector, or on several vectors. They may also be integrated in the genome, on one chromosome or even together in one location, or they may be integrated on different chromosomes. It is also possible to have some sequences on one or more vectors, and some integrated in the genome.

The nucleic acid construct comprises a polynucleotide encoding an acetyl-CoA acetyltransferase (EC 2.3.1.9) which is as described herein elsewhere. In some embodiments, the polynucleotide encoding the acetyl-CoA acetyltransferase encodes GHH_c20420, such as set forth in SEQ ID NO: 1, or a functional variant thereof having at least 70% homology, similarity or identity thereto. In some embodiments, said polynucleotide comprises or consists of SEQ ID NO: 30, or a homologue thereof having at least 70% homology, similarity or identity thereto, which encodes an enzyme which retains acetyl-CoA acetyltransferase activity.

In some embodiments, the polynucleotide encoding the acetyl-CoA acetyltransferase encodes Slip_0499, such as set forth in SEQ ID NO: 2, or a functional variant thereof having at least 70% homology, similarity or identity thereto. In some embodiments, said polynucleotide comprises or consists of SEQ ID NO: 31, or a homologue thereof having at least 70% homology, similarity or identity thereto, which encodes an enzyme which retains acetyl-CoA acetyltransferase activity.

In some embodiments, the polynucleotide encoding the acetyl-CoA acetyltransferase encodes Caur 1461, such as set forth in SEQ ID NO: 3, or a functional variant thereof having at least 70% homology, similarity or identity thereto. In some embodiments, said polynucleotide comprises or consists of SEQ ID NO: 32 or SEQ ID NO: 63, or a homologue thereof having at least 70% homology, similarity or identity thereto, which encodes an enzyme which retains acetyl-CoA acetyltransferase activity.

In some embodiments, the polynucleotide encoding the acetyl-CoA acetyltransferase encodes Slip_0479, such as set forth in SEQ ID NO: 4, or a functional variant thereof having at least 70% homology, similarity or identity thereto. In some embodiments, said polynucleotide comprises or consists of SEQ ID NO: 33, or a homologue thereof having at least 70% homology, similarity or identity thereto, which encodes an enzyme which retains acetyl-CoA acetyltransferase activity.

In some embodiments, the polynucleotide encoding the acetyl-CoA acetyltransferase encodes Dde1, such as set forth in SEQ ID NO: 59, or a functional variant thereof having at least 70% homology, similarity or identity thereto. In some embodiments, said polynucleotide comprises or consists of SEQ ID NO: 61, or a homologue thereof having at least 70% homology, similarity or identity thereto, which encodes an enzyme which retains acetyl-CoA acetyltransferase activity.

In some embodiments, the polynucleotide encoding the acetyl-CoA acetyltransferase encodes Rxy2, such as set forth in SEQ ID NO: 60, or a functional variant thereof having at least 70% homology, similarity or identity thereto. In some embodiments, said polynucleotide comprises or consists of SEQ ID NO: 62, or a homologue thereof having at least 70% homology, similarity or identity thereto, which encodes an enzyme which retains acetyl-CoA acetyltransferase activity.

In some embodiments, the polynucleotide encoding the acetyl-CoA acetyltransferase encodes Tfu_1520, such as set forth in SEQ ID NO: 5, or a functional variant thereof having at least 70% homology, similarity or identity thereto. In some embodiments, said polynucleotide comprises or consists of SEQ ID NO: 34, or a homologue thereof having at least 70% homology, similarity or identity thereto, which encodes an enzyme which retains acetyl-CoA acetyltransferase activity.

In some embodiments, the polynucleotide encoding the acetyl-CoA acetyltransferase encodes Tfu_0436, such as set forth in SEQ ID NO: 6, or a functional variant thereof having at least 70% homology, similarity or identity thereto. In some embodiments, said polynucleotide comprises or consists of SEQ ID NO: 35, or a homologue thereof having at least 70% homology, similarity or identity thereto, which encodes an enzyme which retains acetyl-CoA acetyltransferase activity.

In some embodiments, the polynucleotide encoding the acetyl-CoA acetyltransferase encodes Slip_0880, such as set forth in SEQ ID NO: 7, or a functional variant thereof having at least 70% homology, similarity or identity thereto. In some embodiments, said polynucleotide comprises or consists of SEQ ID NO: 36, or a homologue thereof having at least 70% homology, similarity or identity thereto, which encodes an enzyme which retains acetyl-CoA acetyltransferase activity.

In some embodiments, the polynucleotide encoding the acetyl-CoA acetyltransferase encodes Tfu_2394, such as set forth in SEQ ID NO: 8, or a functional variant thereof having at least 70% homology, similarity or identity thereto. In some embodiments, said polynucleotide comprises or consists of SEQ ID NO: 37, or a homologue thereof having at least 70% homology, similarity or identity thereto, which encodes an enzyme which retains acetyl-CoA acetyltransferase activity.

In some embodiments, the polynucleotide encoding the acetyl-CoA acetyltransferase encodes Slip_1236, such as set forth in SEQ ID NO: 9, or a functional variant thereof having at least 70% homology, similarity or identity thereto. In some embodiments, said polynucleotide comprises or consists of SEQ ID NO: 38, or a homologue thereof having at least 70% homology, similarity or identity thereto, which encodes an enzyme which retains acetyl-CoA acetyltransferase activity.

In some embodiments, the polynucleotide encoding the acetyl-CoA acetyltransferase encodes Caur_1540, such as set forth in SEQ ID NO: 10, or a functional variant thereof having at least 70% homology, similarity or identity thereto. In some embodiments, said polynucleotide comprises or consists of SEQ ID NO: 39, or a homologue thereof having at least 70% homology, similarity or identity thereto, which encodes an enzyme which retains acetyl-CoA acetyltransferase activity.

In some embodiments, the polynucleotide encoding the acetyl-CoA acetyltransferase encodes Tfu_0253, such as set forth in SEQ ID NO: 11, or a functional variant thereof having at least 70% homology, similarity or identity thereto. In some embodiments, said polynucleotide comprises or consists of SEQ ID NO: 40, or a homologue thereof having at least 70% homology, similarity or identity thereto, which encodes an enzyme which retains acetyl-CoA acetyltransferase activity.

In some embodiments, the polynucleotide encoding the acetyl-CoA acetyltransferase encodes CHY_1604, such as set forth in SEQ ID NO: 14, or a functional variant thereof having at least 70% homology, similarity or identity thereto. In some embodiments, said polynucleotide comprises or consists of SEQ ID NO: 43, or a homologue thereof having at least 70% homology, similarity or identity thereto, which encodes an enzyme which retains acetyl-CoA acetyltransferase activity.

In some embodiments, the polynucleotide encoding the acetyl-CoA acetyltransferase encodes CHY_1288, such as set forth in SEQ ID NO: 15, or a functional variant thereof having at least 70% homology, similarity or identity thereto. In some embodiments, said polynucleotide comprises or consists of SEQ ID NO: 44, or a homologue thereof having at least 70% homology, similarity or identity thereto, which encodes an enzyme which retains acetyl-CoA acetyltransferase activity.

In some embodiments, the polynucleotide encoding the acetyl-CoA acetyltransferase encodes Slip_2085, such as set forth in SEQ ID NO: 16, or a functional variant thereof having at least 70% homology, similarity or identity thereto. In some embodiments, said polynucleotide comprises or consists of SEQ ID NO: 45, or a homologue thereof having at least 70% homology, similarity or identity thereto, which encodes an enzyme which retains acetyl-CoA acetyltransferase activity.

In some embodiments, the polynucleotide encoding the acetyl-CoA acetyltransferase encodes Slip_0465, such as set forth in SEQ ID NO: 17, or a functional variant thereof having at least 70% homology, similarity or identity thereto. In some embodiments, said polynucleotide comprises or consists of SEQ ID NO: 46, or a homologue thereof having at least 70% homology, similarity or identity thereto, which encodes an enzyme which retains acetyl-CoA acetyltransferase activity.

In some embodiments, the polynucleotide encoding the acetyl-CoA acetyltransferase encodes Dde1, such as set forth in SEQ ID NO: 59, or a functional variant thereof having at least 70% homology, similarity or identity thereto. In some embodiments, said polynucleotide comprises or consists of SEQ ID NO: 61, or a homologue thereof having at least 70% homology, similarity or identity thereto, which encodes an enzyme which retains acetyl-CoA acetyltransferase activity.

In some embodiments, the polynucleotide encoding the acetyl-CoA acetyltransferase encodes Rxy2, such as set forth in SEQ ID NO: 60, or a functional variant thereof having at least 70% homology, similarity or identity thereto. In some embodiments, said polynucleotide comprises or consists of SEQ ID NO: 62, or a homologue thereof having at least 70% homology, similarity or identity thereto, which encodes an enzyme which retains acetyl-CoA acetyltransferase activity.

In some embodiments, the polynucleotide encoding the acetyl-CoA acetyltransferase encodes CHY_1355, such as set forth in SEQ ID NO: 18, or a functional variant thereof having at least 70% homology, similarity or identity thereto. In some embodiments, said polynucleotide comprises or consists of SEQ ID NO: 47, or a homologue thereof having at least 70% homology, similarity or identity thereto, which encodes an enzyme which retains acetyl-CoA acetyltransferase activity.

In preferred embodiments, the polynucleotide encoding the acetyl-CoA acetyltransferase encodes CHY_1288 (SEQ ID NO: 15), CHY_1355 (SEQ ID NO: 18), Caur_1540 (SEQ ID NO: 10), GHH_c20420 (SEQ ID NO: 1), Caur 1461 (SEQ ID NO: 3), Dde1 (SEQ ID NO: 59), Rxy2 (SEQ ID NO: 60) or Slip_0880 (SEQ ID NO: 7), or a functional variant thereof having at least 70% homology, similarity or identity thereto. Thus in some embodiments, the polynucleotide encoding the acetyl-CoA acetyltransferase comprises or consists of SEQ ID NO: 44, SEQ ID NO: 47, SEQ ID NO: 39, SEQ ID NO: 30, SEQ ID NO: 32, SEQ ID NO: 63, SEQ ID NO: 61, SEQ ID NO: 62 or SEQ ID NO: 36, or homologues thereof having at least 70% homology, similarity or identity thereto encoding enzymes which retain acetyl-CoA acetyltransferase activity. In particular embodiments, the polynucleotide encoding the acetyl-CoA acetyltransferase encodes Caur_1461 (SEQ ID NO: 3), Dde1 (SEQ ID NO: 59) or Slip_0880 (SEQ ID NO: 7), or a functional variant thereof having at least 70% homology, similarity or identity thereto. Thus in some embodiments, the polynucleotide encoding the acetyl-CoA acetyltransferase comprises or consists of SEQ ID NO: 32, SEQ ID NO: 61 or SEQ ID NO: 36, or homologues thereof having at least 70% homology, similarity or identity thereto encoding enzymes which retain acetyl-CoA acetyltransferase activity.

In other preferred embodiments, the polynucleotide encoding the acetyl-CoA acetyltransferase encodes GHH_c20420 (SEQ ID NO: 1), Slip_0499 (SEQ ID NO: 2), Caur_1461 (SEQ ID NO: 3), Dde1 (SEQ ID NO: 59), Rxy2 (SEQ ID NO: 60), Slip_0479 (SEQ ID NO: 4), Tfu_1520 (SEQ ID NO: 5), or Tfu_0436 (SEQ ID NO: 6), or a functional variant thereof having at least 70% homology, similarity or identity thereto. Thus in some embodiments, the polynucleotide encoding the acetyl-CoA acetyltransferase comprises or consists of SEQ ID NO: 30, SEQ ID NO: 31, SEQ ID NO: 32, SEQ ID NO: 63, SEQ ID NO: 33, SEQ ID NO: 34, SEQ ID NO: 61, SEQ ID NO: 62 or SEQ ID NO: 35, or homologues thereof having at least 70% homology, similarity or identity thereto encoding enzymes which retain acetyl-CoA acetyltransferase activity. In particular embodiments, the polynucleotide encoding the acetyl-CoA acetyltransferase encodes Caur_1461 (SEQ ID NO: 3) or Dde1 (SEQ ID NO: 59), or a functional variant thereof having at least 70% homology, similarity or identity thereto. Thus in some embodiments, the polynucleotide encoding the acetyl-CoA acetyltransferase comprises or consists of SEQ ID NO: 32, SEQ ID NO: 63, SEQ ID NO: 33 or SEQ ID NO: 61, or homologues thereof having at least 70% homology, similarity or identity thereto encoding enzymes which retain acetyl-CoA acetyltransferase activity.

The nucleic acid construct further comprises a polynucleotide encoding a second enzyme selected from an acetate CoA transferase, a 3-oxoacid CoA transferase, an acyl CoA:acetate/3-ketoacid CoA-transferase, and an acyl-CoA thioesterase II. In some embodiments, the second enzyme is Tle2. This enzyme consists of two subunits; the polynucleotide encoding Tle2 thus preferably encodes both subunits A and B of Tle2 as set forth in SEQ ID NO: 19 and SEQ ID NO: 20, respectively. In some embodiments, the polynucleotide comprises or consists of SEQ ID NO: 48 and SEQ ID NO: 49, or homologues thereof having at least 70% homology, similarity or identity thereto, which encode subunits which together retain acetyl-CoA transferase activity.

In some embodiments, the polynucleotide encoding the second enzyme encodes Dde2, such as set forth in SEQ ID NO: 21, or a functional variant thereof having at least 70% homology, similarity or identity thereto. In some embodiments, said polynucleotide comprises or consists of SEQ ID NO: 50, or a homologue thereof having at least 70% homology, similarity or identity thereto, which encodes an enzyme which retains 3-oxoacid transferase activity.

In some embodiments, the second enzyme is Ghh2. This enzyme consists of two subunits; the polynucleotide encoding Ghh2 thus preferably encodes both subunits A and B of Ghh2 as set forth in SEQ ID NO: 22 and SEQ ID NO: 23, respectively. In some embodiments, the polynucleotide comprises or consists of SEQ ID NO: 51 and SEQ ID NO: 52, or homologues thereof having at least 70% homology, similarity or identity thereto, which encode subunits which together retain 3-oxoacid transferase activity.

In some embodiments, the second enzyme is Tme. This enzyme consists of two subunits; the polynucleotide encoding Tme thus preferably encodes both subunits A and B of Tme as set forth in SEQ ID NO: 24 and SEQ ID NO: 25, respectively. In some embodiments, the polynucleotide comprises or consists of SEQ ID NO: 53 and SEQ ID NO: 54, or homologues thereof having at least 70% homology, similarity or identity thereto, which encode subunits which together retain 3-oxoacid transferase activity.

In some embodiments, the polynucleotide encoding the second enzyme encodes Pth, such as set forth in SEQ ID NO: 26, or a functional variant thereof having at least 70% homology, similarity or identity thereto. In some embodiments, said polynucleotide comprises or consists of SEQ ID NO: 26, or a homologue thereof having at least 70% homology, similarity or identity thereto, which encodes an enzyme which retains acyl CoA:acetate/3-ketoacid CoA-transferase activity.

In some embodiments, the polynucleotide encoding the second enzyme encodes Rma, such as set forth in SEQ ID NO: 27, or a functional variant thereof having at least 70% homology, similarity or identity thereto. In some embodiments, said polynucleotide comprises or consists of SEQ ID NO: 56, or a homologue thereof having at least 70% homology, similarity or identity thereto, which encodes an enzyme which retains acyl-CoA thioesterase activity.

In preferred embodiments, the polynucleotide encodes Tle2 or a functional variant thereof having at least 70% homology, similarity or identity thereto. Thus in preferred embodiments, the polynucleotide encoding the second enzyme comprises or consists of SEQ ID NO: 48 and SEQ ID NO: 49, or homologues thereof having at least 70% homology, similarity or identity thereto.

The nucleic acid construct further comprises a polynucleotide encoding an acetoacetate decarboxylase. Preferably, the acetoacetate decarboxylase is Cac, such as set forth in SEQ ID NO: 28, or a functional variant thereof having at least 70% homology, similarity or identity thereto. In some embodiments said polynucleotide comprises or consists of SEQ ID NO: 57, or a homologue thereof having at least 70% homology, similarity or identity thereto, which encodes an enzyme which retains acetoacetate decarboxylase activity.

The nucleic acid construct may further comprise a polynucleotide encoding an isopropanol dehydrogenase (EC 1.1.1.80). Preferably, the isopropanol dehydrogenase is Tbr such as set forth in SEQ ID NO: 29, or a functional variant thereof having at least 70% homology, similarity or identity thereto. In some embodiments said polynucleotide comprises or consists of SEQ ID NO: 58, or a homologue thereof having at least 70% homology, similarity or identity thereto, which encodes an enzyme which retains isopropanol dehydrogenase activity.

In some embodiments, the nucleic acid construct comprises or consists of:
i) a polynucleotide encoding an acetyl-CoA acetyltransferase (EC 2.3.1.9) or a functional variant thereof having at least 70% homology, similarity or identity thereto, wherein the acetyl-CoA acetyltransferase is selected from CHY_1288 (SEQ ID NO: 15), CHY_1355 (SEQ ID NO: 18), Caur_1540 (SEQ ID NO: 10), GHH_c20420 (SEQ ID NO: 1), Caur 1461 (SEQ ID NO: 3), Dde1 (SEQ ID NO: 59), Rxy2 (SEQ ID NO: 60) and Slip_0880 (SEQ ID NO: 7); preferably the acetyl-CoA acetyltransferase is selected from Caur_1461 (SEQ ID NO: 3), Dde1 (SEQ ID NO: 59) and Slip_0880 (SEQ ID NO: 7);
ii) a polynucleotide encoding Tle2, wherein Tle2 consists of Tle2 subunit A (EC 2.8.3.8) (SEQ ID NO: 19) and Tle2 subunit B (EC 2.8.3.9) (SEQ ID NO: 20), or functional variants thereof having at least 70% homology, similarity or identity thereto, and
iii) a polynucleotide encoding Cac (SEQ ID NO: 28) or a functional variant thereof having at least 70% homology, similarity or identity thereto.

In some embodiments, the nucleic acid construct comprises or consists of:
i) a polynucleotide encoding an acetyl-CoA acetyltransferase (EC 2.3.1.9) or a functional variant thereof having at least 70% homology, similarity or identity thereto, wherein the acetyl-CoA acetyltransferase is selected from CHY_1288 (SEQ ID NO: 15), CHY_1355 (SEQ ID NO: 18), Caur_1540 (SEQ ID NO: 10), GHH_c20420 (SEQ ID NO: 1), Caur 1461 (SEQ ID NO: 3) Dde1 (SEQ ID NO: 59), Rxy2 (SEQ ID NO: 60) and Slip_0880 (SEQ ID NO: 7); preferably the acetyl-CoA acetyltransferase is selected from Caur_1461 (SEQ ID NO: 3), Dde1 (SEQ ID NO: 59) and Slip_0880 (SEQ ID NO: 7);
ii) a polynucleotide encoding Dde2 (EC 2.8.3.5) (SEQ ID NO: 21), or functional variants thereof having at least 70% homology, similarity or identity thereto, and
iii) a polynucleotide encoding Cac (SEQ ID NO: 28) or a functional variant thereof having at least 70% homology, similarity or identity thereto.

In some embodiments, the nucleic acid construct comprises or consists of:
i) a polynucleotide encoding an acetyl-CoA acetyltransferase (EC 2.3.1.9) or a functional variant thereof having at least 70% homology, similarity or identity thereto, wherein the acetyl-CoA acetyltransferase is selected from CHY_1288 (SEQ ID NO: 15), CHY_1355 (SEQ ID NO: 18), Caur_1540 (SEQ ID NO: 10), GHH_c20420 (SEQ ID NO: 1), Caur 1461 (SEQ ID NO: 3), Dde1 (SEQ ID NO: 59), Rxy2 (SEQ ID NO: 60) and Slip_0880 (SEQ ID NO: 7); preferably the acetyl-CoA acetyltransferase is selected from Caur_1461 (SEQ ID NO: 3), Dde1 (SEQ ID NO: 59) and Slip_0880 (SEQ ID NO: 7);
ii) a polynucleotide encoding Ghh2 (EC 2.8.3.5), wherein Ghh2 consists of Ghh2 subunit A (SEQ ID NO: 22) and Ghh2 subunit B (SEQ ID NO: 23), or functional variants thereof having at least 70% homology, similarity or identity thereto, and
iii) a polynucleotide encoding Cac (SEQ ID NO: 28) or a functional variant thereof having at least 70% homology, similarity or identity thereto.

In some embodiments, the nucleic acid construct comprises or consists of:
i) a polynucleotide encoding an acetyl-CoA acetyltransferase (EC 2.3.1.9) or a functional variant thereof having at least 70% homology, similarity or identity thereto, wherein the acetyl-CoA acetyltransferase is selected from CHY_1288 (SEQ ID NO: 15), CHY_1355 (SEQ ID NO: 18), Caur_1540 (SEQ ID NO: 10), GHH_c20420 (SEQ ID NO: 1), Caur 1461 (SEQ ID NO: 3), Dde1 (SEQ ID NO: 59), Rxy2 (SEQ ID NO: 60) and Slip_0880 (SEQ ID NO: 7); preferably the acetyl-CoA acetyltransferase is selected from Caur_1461 (SEQ ID NO: 3), Dde1 (SEQ ID NO: 59) and Slip_0880 (SEQ ID NO: 7);
ii) a polynucleotide encoding Tme (EC 2.8.3.8), wherein Tme consists of Tme subunit A (SEQ ID NO: 24) and Tme subunit B (SEQ ID NO: 25), or functional variants thereof having at least 70% homology, similarity or identity thereto, and
iii) a polynucleotide encoding Cac (SEQ ID NO: 28) or a functional variant thereof having at least 70% homology, similarity or identity thereto.

In some embodiments, the nucleic acid construct comprises or consists of:
i) a polynucleotide encoding an acetyl-CoA acetyltransferase (EC 2.3.1.9) or a functional variant thereof having at least 70% homology, similarity or identity thereto, wherein the acetyl-CoA acetyltransferase is selected from CHY_1288 (SEQ ID NO: 15), CHY_1355 (SEQ ID NO: 18), Caur_1540 (SEQ ID NO: 10), GHH_c20420 (SEQ ID NO: 1), Caur 1461 (SEQ ID NO: 3), Dde1 (SEQ ID NO: 59), Rxy2 (SEQ ID NO: 60) and Slip_0880 (SEQ ID NO: 7); preferably the acetyl-CoA acetyltransferase is selected from Caur_1461 (SEQ ID NO: 3), Dde1 (SEQ ID NO: 59) and Slip_0880 (SEQ ID NO: 7);
ii) a polynucleotide encoding Pth (EC 2.8.3.1) (SEQ ID NO: 26), or functional variants thereof having at least 70% homology, similarity or identity thereto, and
iii) a polynucleotide encoding Cac (SEQ ID NO: 28) or a functional variant thereof having at least 70% homology, similarity or identity thereto.

In some embodiments, the nucleic acid construct comprises or consists of:
i) a polynucleotide encoding an acetyl-CoA acetyltransferase (EC 2.3.1.9) or a functional variant thereof having at least 70% homology, similarity or identity thereto, wherein the acetyl-CoA acetyltransferase is selected from CHY 1288 (SEQ ID NO: 15), CHY_1355 (SEQ ID NO: 18), Caur_1540 (SEQ ID NO: 10), GHH_c20420 (SEQ ID NO: 1), Caur 1461 (SEQ ID NO: 3), Dde1 (SEQ ID NO: 59), Rxy2 (SEQ ID NO: 60) and Slip_0880 (SEQ ID NO: 7); preferably the acetyl-CoA acetyltransferase is selected from Caur_1461 (SEQ ID NO: 3), Dde1 (SEQ ID NO: 59) and Slip_0880 (SEQ ID NO: 7);
ii) a polynucleotide encoding Rma (EC 3.1.2.-) (SEQ ID NO: 27), or functional variants thereof having at least 70% homology, similarity or identity thereto, and
iii) a polynucleotide encoding Cac (SEQ ID NO: 28) or a functional variant thereof having at least 70% homology, similarity or identity thereto.

In some embodiments, the nucleic acid construct comprises or consists of:
i) a polynucleotide encoding GHH_c20420 (SEQ ID NO: 1), Slip_0499 (SEQ ID NO: 2), Caur_1461 (SEQ ID NO: 3), Dde1 (SEQ ID NO: 59), Rxy2 (SEQ ID NO: 60), Slip_0479 (SEQ ID NO: 4), Tfu_1520 (SEQ ID NO: 5), or Tfu_0436 (SEQ ID NO: 6), or a functional variant thereof having at least 70% homology, similarity or identity thereto; preferably the acetyl-CoA acetyltransferase is selected from Caur_1461 (SEQ ID NO: 3), Dde1 (SEQ ID NO: 59) and Slip_0880 (SEQ ID NO: 7);
ii) a polynucleotide encoding Tle2, wherein Tle2 consists of Tle2 subunit A (EC 2.8.3.8) (SEQ ID NO: 19) and Tle2 subunit B (EC 2.8.3.9) (SEQ ID NO: 20), or functional variants thereof having at least 70% homology, similarity or identity thereto, and
iii) a polynucleotide encoding Cac (SEQ ID NO: 28) or a functional variant thereof having at least 70% homology, similarity or identity thereto.

In some embodiments, the nucleic acid construct comprises or consists of:
i) a polynucleotide encoding GHH_c20420 (SEQ ID NO: 1), Slip_0499 (SEQ ID NO: 2), Caur_1461 (SEQ ID NO: 3), Dde1 (SEQ ID NO: 59), Rxy2 (SEQ ID NO: 60), Slip_0479 (SEQ ID NO: 4), Tfu_1520 (SEQ ID NO: 5), or Tfu_0436 (SEQ ID NO: 6), or a functional variant thereof having at least 70% homology, similarity or identity thereto; preferably the acetyl-CoA acetyltransferase is selected from Caur_1461 (SEQ ID NO: 3), Dde1 (SEQ ID NO: 59) and Slip_0880 (SEQ ID NO: 7);
ii) a polynucleotide encoding Dde2 (EC 2.8.3.5) (SEQ ID NO: 21), or functional variants thereof having at least 70% homology, similarity or identity thereto, and
iii) a polynucleotide encoding Cac (SEQ ID NO: 28) or a functional variant thereof having at least 70% homology, similarity or identity thereto.

In some embodiments, the nucleic acid construct comprises or consists of:
i) a polynucleotide encoding GHH_c20420 (SEQ ID NO: 1), Slip_0499 (SEQ ID NO: 2), Caur_1461 (SEQ ID NO: 3), Dde1 (SEQ ID NO: 59), Rxy2 (SEQ ID NO: 60), Slip_0479 (SEQ ID NO: 4), Tfu_1520 (SEQ ID NO: 5), or Tfu_0436 (SEQ ID NO: 6), or a functional variant thereof having at least 70% homology, similarity or identity thereto; preferably the acetyl-CoA acetyltransferase is selected from Caur_1461 (SEQ ID NO: 3), Dde1 (SEQ ID NO: 59) and Slip_0880 (SEQ ID NO: 7);
ii) a polynucleotide encoding Ghh2 (EC 2.8.3.5), wherein Ghh2 consists of Ghh2 subunit A (SEQ ID NO: 22) and Ghh2 subunit B (SEQ ID NO: 23), or functional variants thereof having at least 70% homology, similarity or identity thereto, and
iii) a polynucleotide encoding Cac (SEQ ID NO: 28) or a functional variant thereof having at least 70% homology, similarity or identity thereto.

In some embodiments, the nucleic acid construct comprises or consists of:
i) a polynucleotide encoding GHH_c20420 (SEQ ID NO: 1), Slip_0499 (SEQ ID NO: 2), Caur_1461 (SEQ ID NO: 3), Dde1 (SEQ ID NO: 59), Rxy2 (SEQ ID NO: 60), Slip_0479 (SEQ ID NO: 4), Tfu_1520 (SEQ ID NO: 5), or Tfu_0436 (SEQ ID NO: 6), or a functional variant thereof having at least 70% homology, similarity or identity thereto; preferably the acetyl-CoA acetyltransferase is selected from Caur_1461 (SEQ ID NO: 3), Dde1 (SEQ ID NO: 59) and Slip_0880 (SEQ ID NO: 7);
ii) a polynucleotide encoding Tme (EC 2.8.3.8), wherein Tme consists of Tme subunit A (SEQ ID NO: 24) and Tme subunit B (SEQ ID NO: 25), or functional variants thereof having at least 70% homology, similarity or identity thereto, and
iii) a polynucleotide encoding Cac (SEQ ID NO: 28) or a functional variant thereof having at least 70% homology, similarity or identity thereto.

In some embodiments, the nucleic acid construct comprises or consists of:
i) a polynucleotide encoding GHH_c20420 (SEQ ID NO: 1), Slip_0499 (SEQ ID NO: 2), Caur_1461 (SEQ ID NO: 3), Dde1 (SEQ ID NO: 59), Rxy2 (SEQ ID NO: 60), Slip_0479 (SEQ ID NO: 4), Tfu_1520 (SEQ ID NO: 5), or Tfu_0436 (SEQ ID NO: 6), or a functional variant thereof having at least 70% homology, similarity or identity thereto; preferably the acetyl-CoA acetyltransferase is selected from Caur_1461 (SEQ ID NO: 3), Dde1 (SEQ ID NO: 59) and Slip_0880 (SEQ ID NO: 7);
ii) a polynucleotide encoding Pth (EC 2.8.3.1) (SEQ ID NO: 26), or functional variants thereof having at least 70% homology, similarity or identity thereto, and
iii) a polynucleotide encoding Cac (SEQ ID NO: 28) or a functional variant thereof having at least 70% homology, similarity or identity thereto.

In some embodiments, the nucleic acid construct comprises or consists of:
i) a polynucleotide encoding GHH_c20420 (SEQ ID NO: 1), Slip_0499 (SEQ ID NO: 2), Caur_1461 (SEQ ID NO: 3), Dde1 (SEQ ID NO: 59), Rxy2 (SEQ ID NO: 60), Slip_0479 (SEQ ID NO: 4), Tfu_1520 (SEQ ID NO: 5), or Tfu_0436 (SEQ ID NO: 6), or a functional variant thereof having at least 70% homology, similarity or identity thereto; preferably the acetyl-CoA acetyltransferase is selected from Caur_1461 (SEQ ID NO: 3), Dde1 (SEQ ID NO: 59) and Slip_0880 (SEQ ID NO: 7);
ii) a polynucleotide encoding Rma (EC 3.1.2.-) (SEQ ID NO: 27), or functional variants thereof having at least 70% homology, similarity or identity thereto, and
iii) a polynucleotide encoding Cac (SEQ ID NO: 28) or a functional variant thereof having at least 70% homology, similarity or identity thereto.

In some embodiments, the nucleic acid construct comprises or consists of:
i) SEQ ID NO: 61, SEQ ID NO: 50 and SEQ ID NO: 57; or
ii) SEQ ID NO: 32, SEQ ID NO: 48 and SEQ ID NO: 49, and SEQ ID NO: 57; or
iii) SEQ ID NO: 36, SEQ ID NO: 48 and SEQ ID NO: 49, and SEQ ID NO: 57;
   and optionally SEQ ID NO: 58,
   or homologues thereof having at least 70% identity or similarity thereto.

In some embodiments, the nucleic acid construct comprises or consists of:
i) SEQ ID NO: 32, SEQ ID NO: 48 and SEQ ID NO: 49, and SEQ ID NO: 57; or
ii) SEQ ID NO: 30, SEQ ID NO: 48 and SEQ ID NO: 49, and SEQ ID NO: 57; or
iii) SEQ ID NO: 31, SEQ ID NO: 48 and SEQ ID NO: 49, and SEQ ID NO: 57; or
iv) SEQ ID NO: 33, SEQ ID NO: 48 and SEQ ID NO: 49, and SEQ ID NO: 57;
   and optionally SEQ ID NO: 58,
   or homologues thereof having at least 70% identity or similarity thereto.

Any of the above described nucleic acid constructs may further comprise a polynucleotide encoding an isopropanol dehydrogenase, preferably Tbr (SEQ ID NO: 29) or a functional variant thereof having at least 70% homology, similarity or identity thereto.

The term “at least 70% homology, similarity or identity” in relation to a nucleic acid sequence is herein to be understood as referring to homologues of a given nucleic acid sequence having at least 70% homology, similarity or identity to said nucleic acid sequence, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology, similarity or identity thereto, which still encode an enzyme retaining the activity of the enzyme encoded by said given nucleic acid sequence. How to test for the relevant activities has been described herein above.

The term “at least 70% homology, similarity or identity” in relation to a protein or enzyme is herein to be understood as referring to homologues of a given protein or enzyme having at least 70% homology, similarity or identity to said protein or enzyme, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology, similarity or identity thereto, which preferably retain at least some of the activity of the original protein or enzyme. How to test for the relevant activities has been described herein above.

All nucleic acid sequences may have been codon-optimised for expression in the microorganism, as is known in the art.

It may be of interest to take advantage of inducible promoters. Thus in some embodiments, the nucleic acid constructs comprises one or more of the above nucleic acid sequences under the control of an inducible promoter. This allows more control of when the enzyme encoded by the sequence is actually expressed, and can be advantageous for example if production of one of the volatile compounds negatively affects cell growth. The skilled person will have no difficulty in identifying suitable inducible promoters. In other embodiments, the nucleic acid constructs are under the control of a constitutive promoter. Such constitutive promoters may be strong promoters.

In some embodiments, the nucleic acid construct is one or more vectors, for example integrative or replicative vectors, such as a plurality of vectors which together form the nucleic acid construct. Suitable vectors are known in the art and readily available to the skilled person. Accordingly, herein is also provided a vector comprising any of the above nucleic acid constructs.

The above nucleic acid constructs are useful for modifying a thermophilic cell, in particular a cell of a genus selected from: Geobacillus, Thermoanaerobacterium, Thermoanaerobacter, Caldanaerobacter, Bacillus, Thermoclostridium, Anoxybacillus, Caldicellulosiruptor, Moorella, Thermus, Thermotoga, Pseudothermotoga, Chloroflexus, Anaerocellum, Rhodothermus, Sulfolobus, Thermococcus, Pyrococcus and Clostridium. In some embodiments, the thermophilic cell is selected from Geobacillus thermoglucosidasius, Geobacillus toebii, Geobacillus stearothermophilus, Geobacillus thermodenitrificans, Geobacillus kaustophilus, Geobacillus thermoleovorans, Geobacillus thermocatenulatus, Thermoanaerobacterium xylanolyticum, Thermoanaerobacterium saccharotyticum, Thermoanaerobacterium thermosaccharolyticum, Thermoanaerobacter mathranii, Thermoanaerobacter pseudoethanolicus, Thermoanaerobacter brockii, Thermoanaerobacter kivui, Thermoanaerobacter brockii, Caldanaerobacter subterraneus, Clostridium thermocellum, Clostridium thermosuccinogenes, Thermoclostridium stercorarium, Bacillus subtilis, Bacillus licheniformis, Bacillus coagulans, Bacillus smithii, Bacillus methanolicus, Bacillus flavothermus, Anoxybacillus kamchatkensis, Anoxybacillus gonensis, Caldicellulosiruptor bescii, Caldicellulosiruptor saccharolyticus, Caldicellulosiruptor kristjanssonii, Caldicellulosiruptor owensensis, Caldicellulosiruptor lactoaceticus, Moorella thermoacetica, Moorella thermoautotrophica, Thermus thermophilus, Thermus aquaticus, Thermotoga maritima, Pseudothermotoga lettingae, Pseudothermotoga thermarum, Chloroflexus aurantiacus, Anaerocellum thermophilum, Rhodothermus marinus, Sulfolobus acidocaldarius, Sulfolobus islandicus, Sulfolobus solfataricus, Thermococcus barophilus, Thermococcus kodakarensis, Pyrococcus abyssi, Pyrococcus furiosus, preferably the cell is a Geobacillus thermoglucosidasius cell, a Bacillus subtilis cell or a Clostridium thermocellum cell.

Also provided herein is a thermophilic cell comprising the nucleic acid construct described herein above.

Also provided herein is a vector or a system of vectors comprising the nucleic acid construct described herein above.

Also provided herein is a host cell comprising the nucleic acid construct or the vector described herein above. The host cell may be a prokaryote or a eukaryote. In a preferred embodiment, the cell is a prokaryote, such as a bacterial cell, for example Escherichia coli. The host cell may be the thermophilic cell which is capable of producing acetone, butanone and/or isopropanol as described herein.

### Kit of Parts

Also provided herein is a kit comprising the nucleic acid construct, the vector or the thermophilic cell described herein above, and optionally instructions for use.

In some embodiments, the kit comprises the nucleic acid constructs and/or the vectors described herein, and may further comprise the thermophilic cell to be modified. The thermophilic cell may be any of the cells described herein above. The kit may further comprise reagents useful for modifying the yeast cell.

### EXAMPLES

### Example 1—Materials and Methods

### Strains, Plasmids and Media

Bacterial strains and plasmids used in this study are listed in Table 1.

TABLE 1
Strains and plasmids used in this study.

| Strain or plasmid | Description | Reference |
|---|---|---|
| Strains | | |
| E. coli NEB5-alpha | fhuA2 Δ(argF-lacZ)U169 phoA glnV44 | New |
| | Φ80 Δ(lacZ)M15 gyrA96 recA1 relA1 | England |
| | endA1 thi-1 hsdR17 | Biolabs |
| | | (USA) |
| G. thermoglucosidasius | Wild type isolate | Bacillus |
| 2542^{T} | | Genetic Stock |
| | | Center (USA) |
| G. thermoglucosidasius G11 | Pₜₕₗ-Cau-Ghh2-Cac | This work |
| G. thermoglucosidasius G12 | Pₜₕₗ-Dde1-Ghh2-Cac | This work |
| G. thermoglucosidasius G13 | Pₜₕₗ-Rxy2-Ghh2-Cac | This work |
| G. thermoglucosidasius G14 | Pₜₕₗ-GHH_c20420-Ghh2-Cac | This work |
| G. thermoglucosidasius G22 | Pₜₕₗ-Cau-Dde2-Cac | This work |
| G. thermoglucosidasius G23 | Pₜₕₗ-Dde1-Dde2-Cac | This work |
| G. thermoglucosidasius G24 | Pₜₕₗ-Rxy2-Dde2-Cac | This work |
| G. thermoglucosidasius G25 | Pₜₕₗ-GHH_c20420-Dde2-Cac | This work |
| G. thermoglucosidasius G27 | Pₜₕₗ-Cau-Tle2-Cac | This work |
| G. thermoglucosidasius G28 | Pₜₕₗ-Dde1-Tle2-Cac | This work |
| G. thermoglucosidasius G29 | Pₜₕₗ-Rxy2-Tle2-Cac | This work |
| G. thermoglucosidasius G30 | Pₜₕₗ-GHH_c20420-Tle2-Cac | This work |
| G. thermoglucosidasius G31 | Δacx:P1-Dde1-Dde2-Cac | This work |
| G. thermoglucosidasius DDC | Δacx::P2-Dde1-Dde2-Cac | This work |
| G. thermoglucosidasius G32 | Δacx::P5-Dde1-Dde2-Cac | This work |
| G. thermoglucosidasius G33 | Δacx::P7-Dde1-Dde2-Cac | This work |
| G. thermoglucosidasius G34 | Δacx::P10-Dde1-Dde2-Cac | This work |
| G. thermoglucosidasius G35 | Δacx::P13-Dde1-Dde2-Cac | This work |
| G. thermoglucosidasius G36 | Δacx::P15-Dde1-Dde2-Cac | This work |
| G. thermoglucosidasius G37 | Δacx::P16-Dde1-Dde2-Cac | This work |
| G. thermoglucosidasius G38 | Δacx::P17-Dde1-Dde2-Cac | This work |
| G. thermoglucosidasius CTC | Δldh::P3-Cau-Tle2-Cac | This work |
| G. thermoglucosidasius CTCl | Δldh::P3-Cau-Tle2-Cac-Tbr | This work |
| G. thermoglucosidasius G51 | Pₜₕₗ-Slip_0465; Δldh::P7-Tle2-Cac | This work |
| G. thermoglucosidasius G52 | Pₜₕₗ-Slip_0479; Δldh::P7-Tle2-Cac | This work |
| G. thermoglucosidasius G53 | Pₜₕₗ-Slip_0499; Δldh::P7-Tle2-Cac | This work |
| G. thermoglucosidasius G54 | Pₜₕₗ-Slip_0880; Δldh::P7-Tle2-Cac | This work |
| G. thermoglucosidasius G55 | Pₜₕₗ-Slip_1236; Δldh::P7-Tle2-Cac | This work |
| G. thermoglucosidasius G56 | Pₜₕₗ-Slip_1985; Δldh::P7-Tle2-Cac | This work |
| G. thermoglucosidasius G58 | Pₜₕₗ-Slip_2085; Δldh::P7-Tle2-Cac | This work |
| G. thermoglucosidasius G59 | Pₜₕₗ-CHY_1288; Δldh::P7-Tle2-Cac | This work |
| G. thermoglucosidasius G60 | Pₜₕₗ-CHY_1355; Δldh::P7-Tle2-Cac | This work |
| G. thermoglucosidasius G61 | Pₜₕₗ-CHY_1604; Δldh::P7-Tle2-Cac | This work |
| G. thermoglucosidasius G62 | Pₜₕₗ-CHY_1738; Δldh::P7-Tle2-Cac | This work |
| G. thermoglucosidasius G63 | Pₜₕₗ-Caur_0234; Δldh::P7-Tle2-Cac | This work |
| G. thermoglucosidasius G64 | Pₜₕₗ-Caur_1461; Δldh::P7-Tle2-Cac | This work |
| G. thermoglucosidasius G65 | Pₜₕₗ-Caur_1540; Δldh::P7-Tle2-Cac | This work |
| G. thermoglucosidasius G66 | Pₜₕₗ-Caur_3529; Δldh::P7-Tle2-Cac | This work |
| G. thermoglucosidasius G67 | Pₜₕₗ-Tfu_0253; Δldh::P7-Tle2-Cac | This work |
| G. thermoglucosidasius G68 | Pₜₕₗ-Tfu_0436; Δldh::P7-Tle2-Cac | This work |
| G. thermoglucosidasius G69 | Pₜₕₗ-Tfu_1520; Δldh::P7-Tle2-Cac | This work |
| G. thermoglucosidasius G70 | Pₜₕₗ-Tfu_2394; Δldh::P7-Tle2-Cac | This work |
| G. thermoglucosidasius G71 | Pₜₕₗ-Despr_1243; Δldh::P7-Tle2-Cac | This work |
| G. thermoglucosidasius G72 | Pₜₕₗ-Despr_1721; Δldh::P7-Tle2-Cac | This work |
| G. thermoglucosidasius G73 | Pₜₕₗ-Despr_2661; Δldh::P7-Tle2-Cac | This work |
| G. thermoglucosidasius G74 | Pₜₕₗ-GHH_c14700; Δldh::P7-Tle2-Cac | This work |
| G. thermoglucosidasius G75 | Pₜₕₗ-GHH_c20420; Δldh::P7-Tle2-Cac | This work |
| G. thermoglucosidasius G76 | Pₜₕₗ-GHH_c34920; Δldh::P7-Tle2-Cac | This work |
| G. thermoglucosidasius G77 | Pₜₕₗ-HFX; Δldh::P7-Tle2-Cac | This work |
| G. thermoglucosidasius G79 | Pₜₕₗ-Cne_therm; Δldh::P7-Tle2-Cac | This work |
| G. thermoglucosidasius G80 | Pₜₕₗ-Tcur_2087; Δldh::P7-Tle2-Cac | This work |
| G. thermoglucosidasius G81 | Pₜₕₗ-SVA_3859; Δldh::P7-Tle2-Cac | This work |
| G. thermoglucosidasius G82 | Pₜₕₗ-Isop_2005; Δldh::P7-Tle2-Cac | This work |
| G. thermoglucosidasius STC | Δldh::P3-Slip_0880-Tle2-Cac | This work |
| Plasmids | | |
| pMTL61110 | ColE1 and pBC1 ori; Km^{R} E. coli- | Sheng et |
| | Geobacillus shuttle vector | al., 2016 |
| pTEC151 | ColE1 and pBC1 ori; Pₜₕₗ-Cau-Ghh2- | This work |
| | Cac | |
| pTEC152 | ColE1 and pBC1 ori; Pₜₕₗ-Dde1-Ghh2- | This work |
| | Cac | |
| pTEC153 | ColE1 and pBC1 ori; Pₜₕₗ-Rxy2-Ghh2- | This work |
| | Cac | |
| pTEC154 | ColE1 and pBC1 ori; Pₜₕₗ- | This work |
| | GHH_c20420-Ghh2-Cac | |
| pTEC162 | ColE1 and pBC1 ori; Pₜₕₗ-Cau-Dde2- | This work |
| | Cac | |
| pTEC163 | ColE1 and pBC1 ori; Pₜₕₗ-Dde1-Dde2- | This work |
| | Cac | |
| pTEC164 | ColE1 and pBC1 ori; Pₜₕₗ-Rxy2-Dde2- | This work |
| | Cac | |
| pTEC165 | ColE1 and pBC1 ori; Pₜₕₗ- | This work |
| | GHH_c20420-Dde2-Cac | |
| pTEC167 | ColE1 and pBC1 ori; Pₜₕₗ-Cau-Tle2-Cac | This work |
| pTEC168 | ColE1 and pBC1 ori; Pₜₕₗ-Dde1-Tle2- | This work |
| | Cac | |
| pTEC169 | ColE1 and pBC1 ori; Pₜₕₗ-Rxy2-Tle2- | This work |
| | Cac | |
| pTEC170 | ColE1 and pBC1 ori; Pₜₕₗ- | This work |
| | GHH_c20420-Tle2-Cac | |
| pTEC181 | ColE1 and pBC1 ori; Δacx::Dde1- | This work |
| | Dde2-Cac | |
| pTEC186 | ColE1 and pBC1 ori; Δacx::P1-Dde1, | This work |
| | integration of P1 promoter | |
| pTEC187 | ColE1 and pBC1 ori; Δacx::P2-Dde1, | This work |
| | integration of P2 promoter | |
| pTEC188 | ColE1 and pBC1 ori; Δacx::P5-Dde1, | This work |
| | integration of P5 promoter | |
| pTEC189 | ColE1 and pBC1 ori; Δacx::P7-Dde1, | This work |
| | integration of P7 promoter | |
| pTEC190 | ColE1 and pBC1 ori; Δacx::P10-Dde1, | This work |
| | integration of P10 promoter | |
| pTEC191 | ColE1 and pBC1 ori; Δacx::P13-Dde1, | This work |
| | integration of P13 promoter | |
| pTEC192 | ColE1 and pBC1 ori; Δacx::P15-Dde1, | This work |
| | integration of P15 promoter | |
| pTEC193 | ColE1 and pBC1 ori; Δacx::P16-Dde1, | This work |
| | integration of P16 promoter | |
| pTEC194 | ColE1 and pBC1 ori; Δacx::P17-Dde1, | This work |
| | integration of P17 promoter | |
| pTEC241 | ColE1 and pBC1 ori; Δldh::Cau-Tle2- | This work |
| | Cac | |
| pTEC251 | ColE1 and pBC1 ori; Δldh::P3-Cau, | This work |
| | integration of P3 promoter | |
| pTEC281 | ColE1 and pBC1 ori; integration of P7 | This work |
| | promoter upstream Tle2-Cac | |
| pTEC286 | ColE1 and pBC1 ori; Pₜₕₗ-Slip_0465 | This work |
| pTEC287 | ColE1 and pBC1 ori; Pₜₕₗ-Slip_0479 | This work |
| pTEC288 | ColE1 and pBC1 ori; Pₜₕₗ-Slip_0499 | This work |
| pTEC289 | ColE1 and pBC1 ori; Pₜₕₗ-Slip_0880 | This work |
| pTEC290 | ColE1 and pBC1 ori; Pₜₕₗ-Slip_1236 | This work |
| pTEC291 | ColE1 and pBC1 ori; Pₜₕₗ-Slip_1985 | This work |
| pTEC293 | ColE1 and pBC1 ori; Pₜₕₗ-Slip_2085 | This work |
| pTEC294 | ColE1 and pBC1 ori; Pₜₕₗ-CHY_1288 | This work |
| pTEC295 | ColE1 and pBC1 ori; Pₜₕₗ-CHY_1355 | This work |
| pTEC296 | ColE1 and pBC1 ori; Pₜₕₗ-CHY_1604 | This work |
| pTEC297 | ColE1 and pBC1 ori; Pₜₕₗ-CHY_1738 | This work |
| pTEC298 | ColE1 and pBC1 ori; Pₜₕₗ-Caur_0234 | This work |
| pTEC299 | ColE1 and pBC1 ori; Pₜₕₗ-Caur_1461 | This work |
| pTEC300 | ColE1 and pBC1 ori; Pₜₕₗ-Caur_1540 | This work |
| pTEC301 | ColE1 and pBC1 ori; Pₜₕₗ-Caur_3529 | This work |
| pTEC302 | ColE1 and pBC1 ori; Pₜₕₗ-Tfu_0253 | This work |
| pTEC303 | ColE1 and pBC1 ori; Pₜₕₗ-Tfu_0436 | This work |
| pTEC304 | ColE1 and pBC1 ori; Pₜₕₗ-Tfu_1520 | This work |
| pTEC305 | ColE1 and pBC1 ori; Pₜₕₗ-Tfu_2394 | This work |
| pTEC306 | ColE1 and pBC1 ori; Pₜₕₗ-Despr_1243 | This work |
| pTEC307 | ColE1 and pBC1 ori; Pₜₕₗ-Despr_1721 | This work |
| pTEC308 | ColE1 and pBC1 ori; Pₜₕₗ-Despr_2661 | This work |
| pTEC309 | ColE1 and pBC1 ori; Pₜₕₗ-GHH_c14700 | This work |
| pTEC310 | ColE1 and pBC1 ori; Pₜₕₗ-GHH_c20420 | This work |
| pTEC311 | ColE1 and pBC1 ori; Pₜₕₗ-GHH_c34920 | This work |
| pTEC312 | ColE1 and pBC1 ori; Pₜₕₗ-HFX | This work |
| pTEC314 | ColE1 and pBC1 ori; Pₜₕₗ-Cne_therm | This work |
| pTEC315 | ColE1 and pBC1 ori; Pₜₕₗ-Tcur_2087 | This work |
| pTEC316 | ColE1 and pBC1 ori; Pₜₕₗ-SVA_3859 | This work |
| pTEC317 | ColE1 and pBC1 ori; Pₜₕₗ-Isop_2005 | This work |
| pTEC322 | ColE1 and pBC1 ori; Tbr integration | This work |

E. coli cells were grown in lysogeny broth (LB) with 100 μg/mL ampicillin or 6.25 μg/mL kanamycin added when needed. Geobacillus strains were grown in either of several media.

The mTGP (modified from Taylor et al., 2008) medium contained per liter: 17 g tryptone, 3 g soy peptone, 5 g NaCl, 2.5 g K₂HPO₄. After autoclavation, sterile solutions were added to final concentrations: 4 mL/L glycerol, 4 g/L sodium pyruvate, 0.59 mM MgSO₄, 0.91 mM CaCl₂ and 0.04 mM FeSO₄. Tripticase soy agar (TSA) contained per liter: 15 g pancreatic digest of casein, 5 g papaic digest of soybean, 5 g NaCl, 15 g agar. SPY medium consisted of 16 g/l soy peptone, 10 g/l yeast extract, 5 g/l NaCl. Its pH was adjusted to 7.0 by adding 5M NaOH. When indicated, glycerol was added to the final concentration of 10 g/l.

Thermophile minimal medium (TMM) was adapted from Fong et al., 2006, with some modifications. It contained, per liter: Six salts solution (SSS), 930 mL; 1 M MOPS (pH 8.2), 40 mL; 1 mM FeSO₄ in 0.4 M tricine, 10 mL; 0.132 M K₂HPO₄, 10 mL; 0.953 M NH₄Cl, 10 mL; 1 M CaCl₂, 0.5 mL; trace elements solution, 0.5 ml; Wolfe's vitamin solution, 10 mL. SSS contained, per 930 mL: 4.6 g NaCl, 1.35 g Na₂SO₄, 0.23 g KCl, 0.037 g KBr, 1.72 g MgCl₂·6H₂O, 0.83 g NaNO₃. Trace elements solution contained, per liter: 1 g FeCl₃·6H₂O, 0.18 g ZnSO₄·7H₂O, 0.12 g CuCl₂·2H₂O, 0.12 g MnSO₄·H₂O, 0.18 g CoCl₂·6H₂O. Yeast extract in final concentration of 0.05% (w/v) was added when indicated. For Geobacillus spp. selection 12.5 μg/mL kanamycin was used.

### DNA Manipulations

Genomic DNA was extracted using the Wizard® Genomic DNA Purification Kit (Promega) according to producer's specifications. Plasmid extractions were performed using NucleoSpin® Plasmid EasyPure kit (Macherey-Nagel).

### PCR and Cloning

Primers used in this study are described in Table 2.

TABLE 2
Oligonucleotides used in this study

| Primer name | Target sequence | Amplified region |
|---|---|---|
| PNJ23 | pMTL61110 backbone | |
| PNJ24b | pMTL61110 backbone | |
| PNJ560 | Promoter of thl gene (Pₜₕₗ) | AE001437: 3007142 . . . 3007364 |
| PAP0008 | Promoter of thl gene (Pₜₕₗ) | AE001437: 3007142 . . . 3007364 |
| PNJ564 | Cac | AE001438: 179848 . . . 180582 |
| PNJ565 | Cac | AE001438: 179848 . . . 180582 |
| PNJ575 | Cau | Synthetic DNA fragment |
| PNJ576 | Cau | Synthetic DNA fragment |
| PNJ579 | Dde2 | Synthetic DNA fragment |
| PNJ580 | Dde2 | Synthetic DNA fragment |
| PNJ587 | Rxy2 | Synthetic DNA fragment |
| PNJ588 | Rxy2 | Synthetic DNA fragment |
| PNJ601 | Ghh2 | CP004008: 2062407 . . . 2063782 |
| PNJ602 | Ghh2 | CP004008: 2062407 . . . 2063782 |
| PNJ603 | Tle2 | Synthetic DNA fragment |
| PNJ604 | Tle2 | Synthetic DNA fragment |
| PNJ834 | Promoter of groE gene | Synthetic DNA fragment |
| | and its derivatives | |
| PNJ855 | Promoter of groE gene | Synthetic DNA fragment |
| | and its derivatives | |
| PNJ856 | Tbr | Synthetic DNA fragment |
| PNJ865 | Tbr | Synthetic DNA fragment |
| PNJ836 | Cau fragment (750 bp) | Synthetic DNA fragment |
| PNJ1130 | Cau fragment (750 bp) | Synthetic DNA fragment |
| PNJ851 | Δldh left flank 1000 bp | NZ_CP012712: 1231012 . . . 1231993 |
| PNJ852 | Δldh left flank 1000 bp | NZ_CP012712: 1231012 . . . 1231993 |
| PNJ853 | Δldh right flank 1000 bp | NZ_CP012712: 1228849 . . . 1229848 |
| PNJ854 | Δldh right flank 1000 bp | NZ_CP012712: 1228849 . . . 1229848 |
| PAP0011 | GHH_c14700 | NC_020210.1: 1479170 . . . 1480348 |
| PAP0012 | GHH_c14700 | NC_020210.1: 1479170 . . . 1480348 |
| PAP0013 | GHH_c16740 | NC_020210.1: 1673461 . . . 1674630 |
| PAP0014 | GHH_c16740 | NC_020210.1: 1673461 . . . 1674630 |
| PAP0015 | GHH_c34920 | NC_020210.1: 3486863 . . . 3488041 |
| PAP0016 | GHH_c34920 | NC_020210.1: 3486863 . . . 3488041 |
| PAP0017 | Slip_0465 | NC_014220.1: 466521 . . . 467774 |
| PAP0018 | Slip_0465 | NC_014220.1: 466521 . . . 467774 |
| PAP0019 | Slip_0479 | NC_014220.1: 485033 . . . 486211 |
| PAP0020 | Slip_0479 | NC_014220.1: 485033 . . . 486211 |
| PAP0021 | Slip_0499 | NC_014220.1: 507904 . . . 509106 |
| PAP0022 | Slip_0499 | NC_014220.1: 507904 . . . 509106 |
| PAP0023 | Slip_0880 | NC_014220.1: 915786 . . . 916970 |
| PAP0024 | Slip_0880 | NC_014220.1: 915786 . . . 916970 |
| PAP0025 | Slip_1236 | NC_014220.1: 1267071 . . . 1268252 |
| PAP0026 | Slip_1236 | NC_014220.1: 1267071 . . . 1268252 |
| PAP0027 | Slip_1985 | NC_014220.1: 2030049 . . . 2031233 |
| PAP0028 | Slip_1985 | NC_014220.1: 2030049 . . . 2031233 |
| PAP0031 | Slip_2085 | NC_014220.1: 2121617 . . . 2122924 |
| PAP0032 | Slip_2085 | NC_014220.1: 2121617 . . . 2122924 |
| PAP0033 | CHY_1288 | NC_007503.1: 1145592 . . . 1146773 |
| PAP0034 | CHY_1288 | NC_007503.1: 1145592 . . . 1146773 |
| PAP0035 | CHY_1355 | NC_007503.1: 1213541 . . . 1214722 |
| PAP0036 | CHY_1355 | NC_007503.1: 1213541 . . . 1214722 |
| PAP0037 | CHY_1604 | NC_007503.1: 1442386 . . . 1443567 |
| PAP0038 | CHY_1604 | NC_007503.1: 1442386 . . . 1443567 |
| PAP0039 | CHY_1738 | NC_007503.1: 1555755 . . . 1556942 |
| PAP0040 | CHY_1738 | NC_007503.1: 1555755 . . . 1556942 |
| PAP0041 | Caur_0234 | NC_010175.1: 295186 . . . 296352 |
| PAP0042 | Caur_0234 | NC_010175.1: 295186 . . . 296352 |
| PAP0043 | Caur_1461 | NC_010175.1: 2018661 . . . 2019848 |
| PAP0044 | Caur_1461 | NC_010175.1: 2018661 . . . 2019848 |
| PAP0045 | Caur_1540 | NC_010175.1: 2116262 . . . 2117443 |
| PAP0046 | Caur_1540 | NC_010175.1: 2116262 . . . 2117443 |
| PAP0053 | GHH_c20420 | NC_020210.1: 2058835 . . . 2060010 |
| PAP0054 | GHH_c20420 | NC_020210.1: 2058835 . . . 2060010 |
| PAP0055 | Tfu_0253 | NC_007333.1: 283496 . . . 284638 |
| PAP0056 | Tfu_0253 | NC_007333.1: 283496 . . . 284638 |
| PAP0057 | Tfu_0436 | NC_007333.1: 493716 . . . 494939 |
| PAP0058 | Tfu_0436 | NC_007333.1: 493716 . . . 494939 |
| PAP0059 | Tfu_1520 | NC_007333.1: 1752203 . . . 1753384 |
| PAP0060 | Tfu_1520 | NC_007333.1: 1752203 . . . 1753384 |
| PAP0061 | Tfu_2394 | NC_007333.1: 2816439 . . . 2817626 |
| PAP0062 | Tfu_2394 | NC_007333.1: 2816439 . . . 2817626 |
| PAP0065 | Despr_1243 | NC_014972.1: 1413019 . . . 1414203 |
| PAP0066 | Despr_1243 | NC_014972.1: 1413019 . . . 1414203 |
| PAP0067 | Despr_1721 | NC_014972.1: 1983716 . . . 1984891 |
| PAP0068 | Despr_1721 | NC_014972.1: 1983716 . . . 1984891 |

PCR of DNA fragments for USER cloning was performed with primers containing uracil using the Phusion U Hot Start DNA Polymerase (Thermo Fisher Scientific). Colony PCR was performed with Taq 2x Master Mix (New England Biolabs) in order to detect positive colonies. Reactions were done according to manufacturers' recommendations with elongation times and annealing temperatures adjusted for specific targets and primers. DNA cloning was performed using USER (uracil-specific excision reagent) technology (Cavaleiro et al., 2015).

PCR-amplified DNA fragments containing a primer-incorporated uracil close to both of their 5′-ends were mixed (purification after PCR was not necessary) and treated with DpnI enzyme (Thermo Fisher Scientific) for 30 min at 37° C. to digest template DNA. USER™ enzyme (New England Biolabs) was then added, and the mixture was incubated in three steps: 1) 37° C. for 15 min; 2) 12° C. for 15 min; 3) 10° C. for 10 min. It was then transferred on ice and mixed with chemically competent E. coli cells.

Transformation of E. coli

Chemically competent E. coli NEB5-alpha cells (New England Biolabs) were transformed according to manufacturer's recommendations.

Transformation of G. thermoglucosidasius

The procedure was based on the protocol described by Taylor et al., 2008, with some steps modified. G. thermoglucosidasius was grown overnight on a TSA agar plate at 60° C. A loopful of cells was inoculated into 50 mL of pre-warmed liquid SPY medium in a 250 ml flask and incubated at 60° C. and 250 rpm until the culture reached OD600 of approximately 2.0. Cells were cooled down on ice for 10 min and harvested by centrifugation at 2600 g for 10 min. They were washed four times (2600 g for 10 min) with freshly prepared ice-cold electroporation buffer. The buffer contained (per 100 mL) 9.1 g mannitol, 9.1 g sorbitol, 10 mL glycerol, and was sterilized by filtration. Buffer was added at the volumes of 25 ml, 15 ml, 15 ml and 10 ml for each consecutive step. After the last washing step, the cell pellet was dissolved in 2 mL of electroporation buffer, distributed in 60 μL aliquots and stored at −80° C. until further use.

For the transformation, an aliquot was thawed on ice and mixed with DNA. It was transferred into an electroporation cuvette with a 1 mm gap between electrodes (Bio-Rad) and subjected to a discharge using the Gene Pulser Xcel™ (Bio-Rad) under following conditions: 2.5 kV, 600 Ω, 10 μF. Time constants typically were 4-5 ms. Immediately after electroporation cells were dissolved in 1 mL of pre-heated SPY medium supplemented with glycerol and recovered at 52° C. for 4 hours at 200 rpm. Afterwards they were spun down and seeded on selective agar media plates.

### DNA Design and Analysis

Codon optimization was done using the online service of Integrated DNA Technologies, Inc. (https://eu.idtdna.com/CodonOpt) or Gene Designer from DNA 2.0 (Villalobos et al., 2006). DNA sequencing was performed by Eurofins Scientific (Luxembourg).

### GC-MS Analysis

Strains expressing acetone pathways were grown in 2 ml of TMM with respective supplements in sterile 20 ml headspace vials. To prevent acetone loss the vials were kept closed during the whole time of culture growth until sampling for chromatography. After 20 hours of incubation cultures were frozen at −20° C. to stop growth and metabolic activity, and transferred for measurement.

Acetone concentrations were measured with analytical GC-MS (Bruker Scion 436 GC TQ) using BP20 capillary column (30 m, internal diameter 0.25 mm, film thickness 0.25 mm). Helium was used as a carrier. The inlet temperature was set to 250° C., and the oven temperature was programmed as follows: run at 37° C. for 5 min, then raised at a rate of 5° C./min until it reached 100° C., followed by the increase at 15° C./min up to 250° C., and finally hold for 3 min. Mass spectrometry was run using electron ionization method. Full scan mode was used with scan range from 35 to 400 amu. Injection volumes were from 1 μl to 5 μl in splitless mode for low acetone concentrations and split mode (1:1) for high concentrations.

### Example 2—Acetone Production in G. thermoglucosidasius

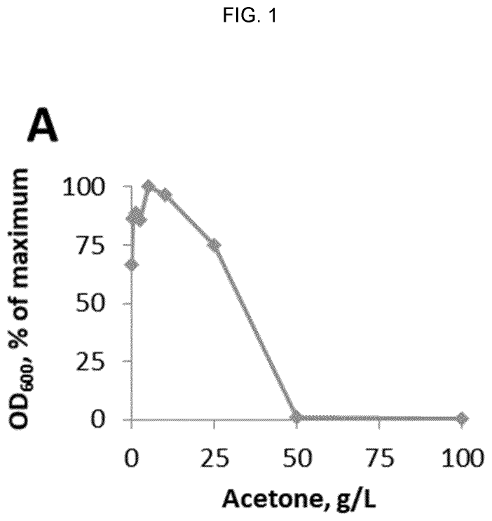

For production of acetone in G. thermoglucosidasius, we initially tested the tolerance of G. thermoglucosidasius towards acetone. When grown in tightly sealed vessels with 1:10 medium to headspace volume ratio, this strain was found to tolerate at least 25 g/l of acetone (FIG. 1). We therefore next sought to express functional acetone pathways in Geobacillus sp. The acetone operons were introduced using the vector backbone to the recently developed pMTL61110 (Sheng et al., 2016).

Combinations of four thiolase variants and three oxoacid CoA-transferase variants were constructed together with acetoacetate decarboxylase from C. acetobutylicum as operons under the control of thiolase promoter from C. acetobutylicum. Strains of G. thermoglucosidasius carrying these operons were grown in: i) semi-defined medium with 1% glucose, and ii) nutritionally rich medium supplemented with 0.2% glucose. Among all combinations, Dde1-Dde2-Cac produced highest titers in semi-defined medium, whereas Cau-Tle2-Cac performed best in rich broth. Results are shown in Table 3.

TABLE 3
Acetone titers (mg/l) by G. thermoglucosidasius carrying different
combinations of thiolase variants (rows) and oxoacid CoA-transferase
variants (columns). In each combination, two enzyme genes were
expressed from plasmid in one operon together with acetoacetate
decarboxylase from Clostridium acetobutylicum
(UniProt ID P23670) under control of promoter of thiolase gene
(AE001437: 3007142 . . . 3007364). Strains were cultivated in either
minimal (upper value in the cell) or in rich medium (lower value).

| | | | | |
|---|---|---|---|---|
| ACoAT | THL | Dde2 | Tle2 | Ghh2 |

| | | | |
|---|---|---|---|
| Cau | 81 | 59 | 8 |
| | 124 | 1043 | 132 |
| Dde1 | 203 | 3 | 17 |
| | 586 | 18 | 73 |
| GHH_c20420 | 38 | 30 | 7 |
| | 128 | 550 | 167 |
| Rxy2 | 73 | 32 | 7 |
| | 125 | 126 | 252 |

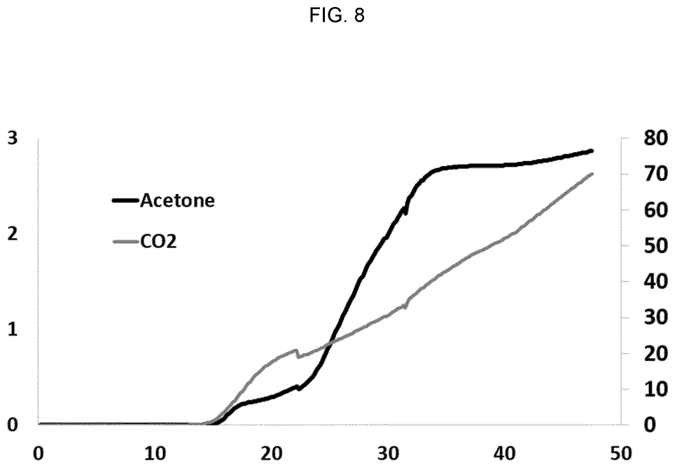

The CTC strain was also tested in a 30 L fed-batch fermentation, fed with 2 g/L/h glucose, 1 g/L/h acetic acid, 1 g/L/h yeast extract. The CTC strain was grown in TMM medium, supplemented with 2% glucose, 0.2% acetic acid, 1% yeast extract. The CTC genes were integrated into the genome under the control of the strong promoter P3 (Pogrebnyakov et al., 2017). Up to 2.9 g/L acetone were achieved (FIG. 8).

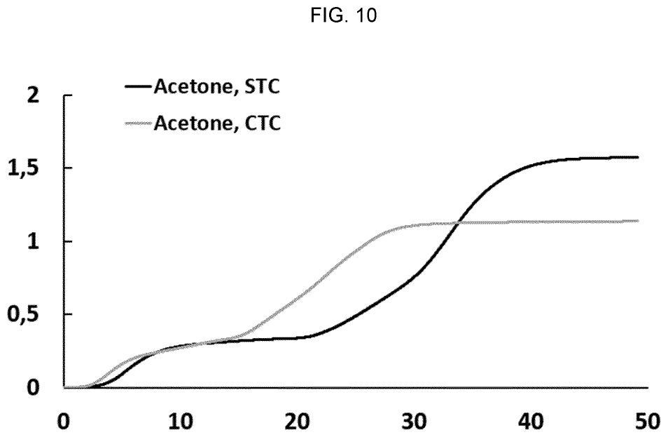

Both the STC strain (Slip_0880-Tle2-Cac) and the CTC strain (Caur_1461-Tle2-Cac) were also tested in a 1 L constant fed-batch fermentation. The strains were grown in TMM medium, supplemented with 2% glucose, 0.2% acetic acid, 1% yeast extract. The CTC and STC genes were integrated into the genome under the control of the strong promoter P3. The STC strain achieved a final acetone titer of 1.6 g/L, while the CTC strain achieved a final acetone titer of 1.1 g/L (FIG. 10). The STC strain reached a higher titer in spite of its growth rate and maximal cell density being lower than the CTC strain (1.5 h⁻¹ and OD600 of 6.9 for STC against 2.1 h⁻¹ and OD600 of 16.5 for CTC). The STC strain also consumed more glucose and acetate than the CTC strain, indicating that it is even more efficient in converting substrates into the desired product (acetone) rather than cell biomass.

### Example 3—Effect of Promoter Strength

Two of the best performing enzyme combinations, Dde1-Dde2-Cac and Cau-Tle2-Cac, where Cau is a codon optimized version of Caur_1461, were overexpressed in G. thermoglucosidasius. Each operon was integrated into the chromosome of G. thermoglucosidasius, replacing either putative acetone carboxylase (AOT13_RS09545, AOT13_RS09550 and AOT13_RS09555) in case of Dde1-Dde2-Cac, or lactate dehydrogenase (AOT13_RS05985) in case of Cau-Tle2-Cac. A range of constitutive promoters with various levels of activity, from low to high (Pogrebnyakov et al., 2017), were integrated upstream of these operons to drive their expression, yielding G. thermoglucosidasius strains G31-G38 and DDC. Acetone titers from Dde1-Dde2-Cac increased with the increasing strength of the promoter (Table 3).

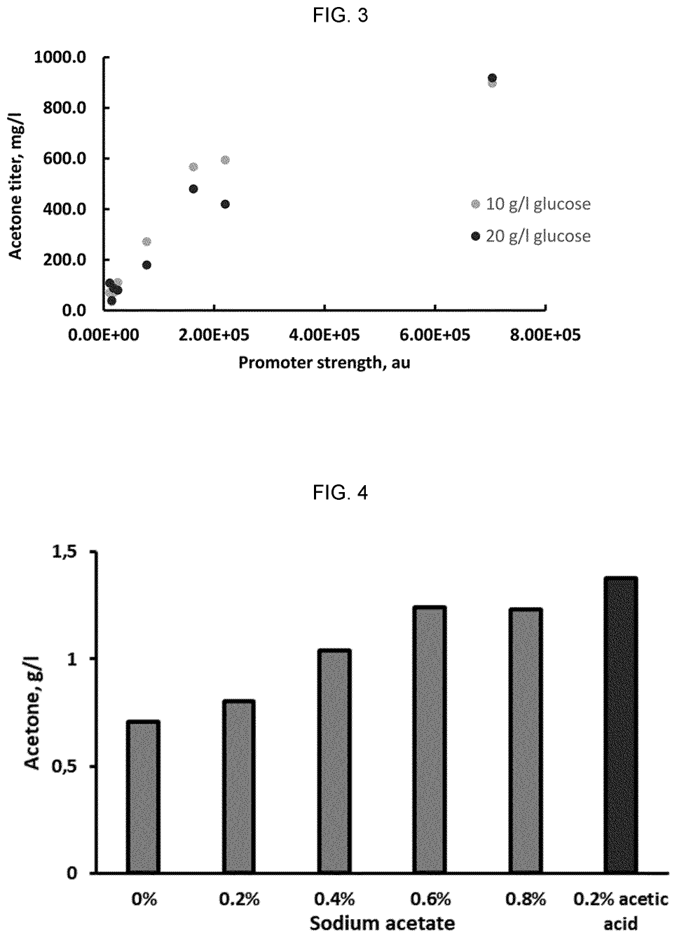

Overexpression of Cau-Tle2-Cac operon in G. thermoglucosidasius strain CTC also led to increase in acetone titers. Addition of sodium acetate and especially acetic acid further increases acetone titers up to two-fold (FIG. 4, Table 4). In nutritionally rich SPY medium supplemented with 0.2% acetic acid strain CTC produced 1.61 g/l acetone.

TABLE 4
Production of acetone by G. thermoglucosidasius
strain CTC in semi-defined medium with 1% glucose
and different concentrations of acetate

| Concentration of additive | Acetone, mg/l |
|---|---|
| 0% | 0.71 |
| 0.2% sodium acetate | 0.80 |
| 0.4% sodium acetate | 1.04 |
| 0.6% sodium acetate | 1.24 |
| 0.8% sodium acetate | 1.23 |
| 0.2% acetic acid | 1.38 |

### Example 4—Effect of Sugar Composition

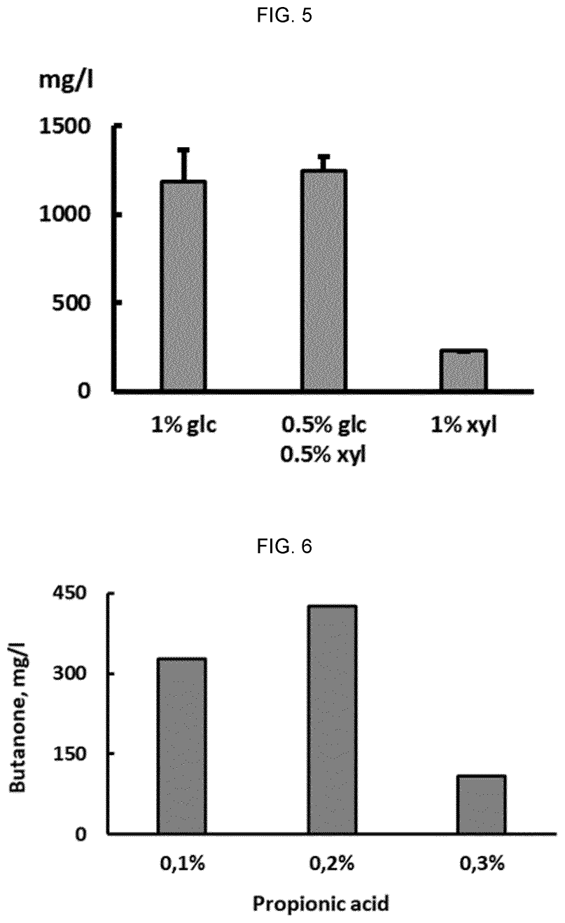

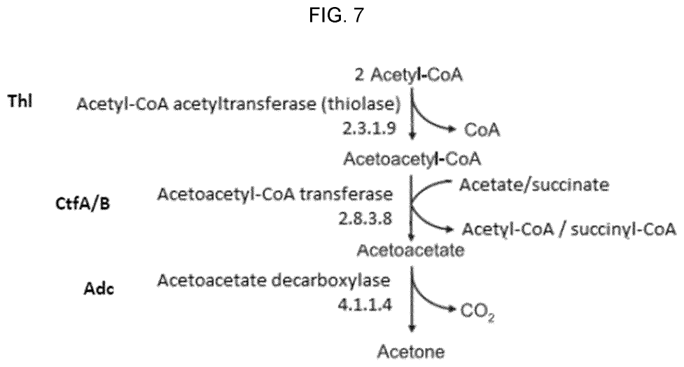

Sugar composition of the substrate also affects acetone production by G. thermoglucosidasius. This species is able to utilize many pentose and hexose sugars, in particular glucose and xylose. G. thermoglucosidasius CTC was grown in the presence of a mixture of these monosaccharides and converted them into high yields of acetone (FIG. 5).

### Example 5—Acetone and Butanone Production in G. thermoglucosidasius

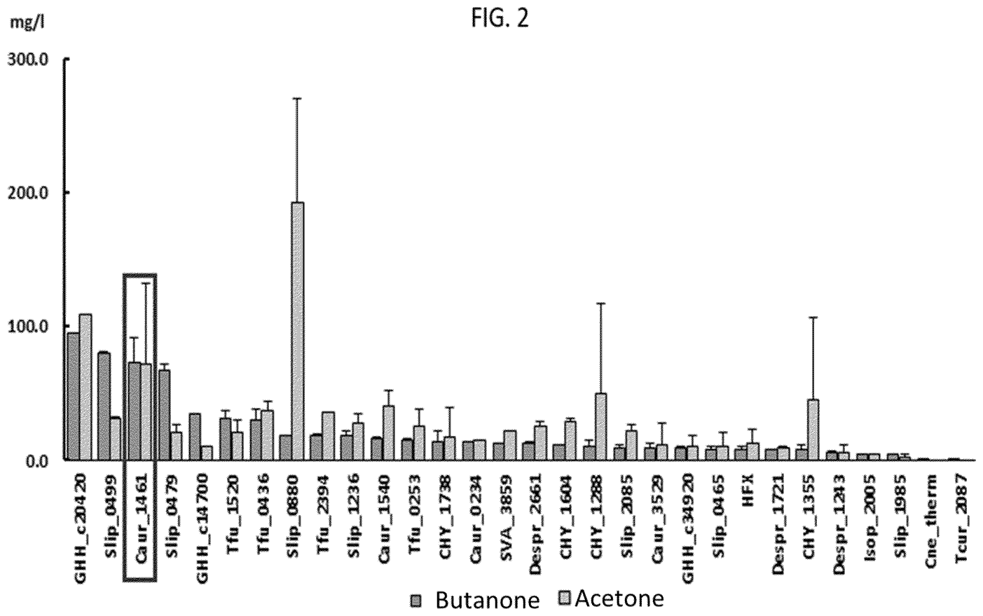

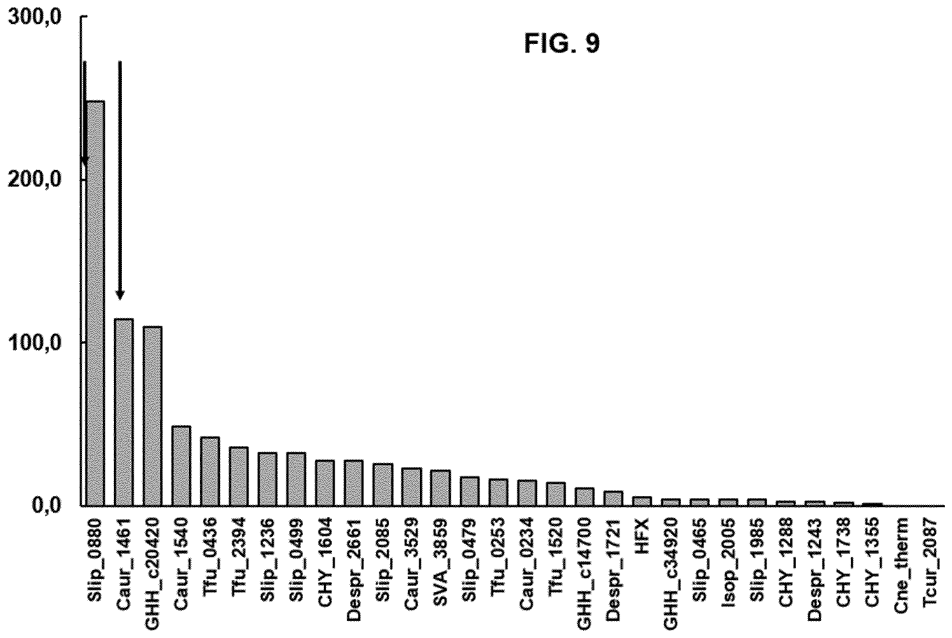

Multiple variants of thiolases and acyl-CoA:acyl-CoA alkyltransferases from thermophilic organisms were screened for the production of butanone and acetone. They were expressed under the control of thiolase promoter from C. acetobutylicum in the strain of G. thermoglucosidasius carrying Tle2 and Cac genes in the chromosome under the control of medium strength promoter P7, created previously (Pogrebnyakov et al., 2017). The resulting strains were named G51-G82. They were grown in semi-defined TMM medium supplemented with 1% glucose and 0.2% propionic acid. In these conditions, most strains produced a mixture of butanone and acetone at different ratios and titers (Table 5). Thiolase variants, which contributed to the highest butanone production, were Caur_1461, GHH_c20420, Slip_0499 and Slip_0479. Expression of variant Slip_0880 resulted in the highest acetone titer with relatively low butanone amounts. Results are shown in FIGS. 2 and 9; the data in FIG. 9 show results from the same experiments as FIG. 2, merged with an additional independent measurement performed under the same conditions.

TABLE 5
production of butanone and acetone in G. thermoglucosidasius
expressing the indicated thiolase, and otherwise expressing
the acetyl CoA transferase Tle2 from Pseudothermotoga lettingae
(UniProt ID A8F7H7, A8F7H6) and the acetoacetate decarboxylase
Cac from Clostridium acetobutylicum (P23670).

| Strain | Thiolase | Butanone, mg/l | Acetone, mg/l |
|---|---|---|---|
| G75 | GHH_c20420 | 95.5 | 109.6 |
| G53 | Slip_0499 | 80.2 | 31.8 |
| G64 | Caur_1461 | 72.6 | 72.3 |
| G52 | Slip_0479 | 67.0 | 21.2 |
| G74 | GHH_c14700 | 34.5 | 10.8 |
| G69 | Tfu_1520 | 30.9 | 20.8 |
| G68 | Tfu_0436 | 30.0 | 37.0 |
| G54 | Slip_0880 | 18.9 | 193.2 |
| G70 | Tfu_2394 | 18.2 | 36.1 |
| G55 | Slip_1236 | 18.0 | 27.8 |
| G65 | Caur_1540 | 15.7 | 40.9 |
| G67 | Tfu_0253 | 15.1 | 24.9 |
| G62 | CHY_1738 | 13.4 | 17.4 |
| G63 | Caur_0234 | 13.3 | 15.5 |
| G81 | SVA_3859 | 12.8 | 21.8 |
| G73 | Despr_2661 | 12.7 | 25.9 |
| G61 | CHY_1604 | 11.3 | 29.4 |
| G59 | CHY_1288 | 9.8 | 50.3 |
| G58 | Slip_2085 | 9.7 | 21.5 |
| G66 | Caur_3529 | 9.5 | 11.6 |
| G76 | GHH_c34920 | 8.8 | 10.1 |
| G51 | Slip_0465 | 8.3 | 10.8 |
| G77 | HFX | 7.9 | 13.0 |
| G72 | Despr_1721 | 7.6 | 9.2 |
| G60 | CHY_1355 | 7.5 | 45.0 |
| G71 | Despr_1243 | 5.3 | 6.2 |
| G82 | Isop_2005 | 4.2 | 4.2 |
| G56 | Slip_1985 | 4.1 | 2.1 |
| G79 | Cne_therm | 1.4 | 0.0 |
| G80 | Tcur_2087 | 1.1 | 0.0 |

G. thermoglucosidasius strain CTC from previous example overexpresses Cau-Tle2-Cac operon, where Cau is a codon optimized version of Caur_1461, one of the best butanone producers in this example. G. thermoglucosidasius CTC was grown in TMM supplemented with 1% glucose and 0.1% to 0.3% propionic acid, and produced up to 0.43 g/l butanone (FIG. 6, Table 6).

TABLE 6
Production of butanone by G. thermoglucosidasius
strain CTC in semi-defined medium with 1% glucose
and different concentrations of propionic acid.

| Propionic acid | Butanone, mg/l |
|---|---|
| 0.1% | 326 |
| 0.2% | 426 |
| 0.3% | 109 |

### Example 6—Isopropanol Production in G. thermoglucosidasius

One enzymatic step involving alcohol dehydrogenase is required to convert acetone into isopropanol. A specific isopropanol dehydrogenase from Thermoanaerobacter brockii has been identified previously (Hanai et al., 2007). Codon optimized version of this gene was integrated into the genome of G. thermoglucosidasius CTC downstream of Cac gene, yielding strain CTCI. G. thermoglucosidasius CTCI grown in TMM supplemented with 1% glucose produced 0.11 g/l isopropanol.

### REFERENCES

“Acetone market: global industry analysis and opportunity assessment, 2014-2020,” 2015
Z. C. Baer, S. Bormann, S. Sreekumar, A. Grippo, F. D. Toste, H. W. Blanch, and D. S.

Clark, “Co-production of acetone and ethanol with molar ratio control enables production of improved gasoline or jet fuel blends,” Biotechnol Bioeng, vol. 9999, no. 10, pp. 1-9, 2016
A. Banerjee, C. Leang, T. Ueki, K. P. Nevin, and D. R. Lovley, “Lactose-inducible system for metabolic engineering of Clostridium ljungdahlii,” Appl Env. Microb, vol. 80, no. 8, pp. 2410-2416, 2014.
L. L. Bermejo, N. E. Welker, and E. T. Papoutsakis, “Expression of Clostridium acetobutylicum ATCC 824 genes in Escherichia coli for acetone production and acetate detoxification,” Appl Env. Microbiol, vol. 64, no. 3, pp. 1079-1085, 1998
I. W. Bogorad, T. Lin, and J. C. Liao, “Synthetic non-oxidative glycolysis enables complete carbon conservation,” Nature, vol. 502, no. 7473, pp. 693-697, 2013
E. F. Bosma, J. Van Der Oost, W. M. De Vos, and R. Van Kranenburg, “Sustainable production of bio-based chemicals by extremophiles,” Curr Biotechnol, vol. 2, pp. 360-379, 2013
A. M. Cavaleiro, S. H. Kim, S. Seppsls, M. T. Nielsen, and M. H. H. Norholm, “Accurate DNA Assembly and Genome Engineering with Optimized Uracil Excision Cloning,” ACS Synth. Biol., vol. 4, no. 9, pp. 1042-1046, 2015
R. E. Cripps, K. Eley, D. J. Leak, B. Rudd, M. Taylor, M. Todd, S. Boakes, S. Martin, and T. Atkinson, “Metabolic engineering of Geobacillus thermoglucosidasius for high yield ethanol production,” Metab. Eng., vol. 11, no. 6, pp. 398-408, 2009
J. C. N. Fong, C. J. Svenson, K. Nakasugi, C. T. C. Leong, J. P. Bowman, B. Chen, D.

R. Glenn, B. A. Neilan, and P. L. Rogers, “Isolation and characterization of two novel ethanol-tolerant facultative-anaerobic thermophilic bacteria strains from waste compost,” Extremophiles, vol. 10, no. 5, pp. 363-372, 2006
T. Hanai, S. Atsumi, and J. C. Liao, “Engineered synthetic pathway for isopropanol production in Escherichia coli,” Appl. Environ. Microbiol., vol. 73, no. 24, pp. 7814-7818, 2007.
M. C. Ho, J. F. Ménétret, H. Tsuruta, and K. N. Allen, “The origin of the electrostatic perturbation in acetoacetate decarboxylase,” Nature, vol. 459, pp. 393-397, 2009.
S. Hoffmeister, M. Gerdom, F. R. Bengelsdorf, S. Linder, S. Flüchter, H. Özturk, W. Blümke, A. May, R. Fischer, H. Bahl, and P. Dürre, “Acetone production with metabolically engineered strains of Acetobacterium woodii,” Metab Eng, vol. 36, pp. 37-47, 2016.
D. T. Jones and D. R. Woods, “Acetone-butanol fermentation revisited,” Microbiol Rev, vol. 50, no. 4, pp. 484-524, 1986
M. Mathieu et al., “The 1.8 Å crystal structure of the dimeric peroxisomal 3-ketoacyl-CoA thiolase of Saccharomyces cerevisiae: Implications for substrate binding and reaction mechanism,” J. Mol. Biol., vol. 273, no. 3, pp. 714-728, 1997
A. May, R.-J. Fischer, M. S. Thum, S. Schaffer, S. Verseck, P. Durre, and H. Bahl, “A modified pathway for the production of acetone in Escherichia coli,” Metab Eng, vol. 15, pp. 218-225, 2013.
I. Pogrebnyakov, C. B. Jendresen, and A. T. Nielsen, “Genetic toolbox for controlled expression of functional proteins in Geobacillus spp.,” PLoS One, vol. 12, no. 2, pp. 1-15, 2017
L. Sheng and N. P. Minton, “Development of the shuttle vector pMTL for transformation in Geobacillus spp. and Escherichia coli,” in print, 2016.
M. P. Taylor, C. D. Esteban, and D. J. Leak, “Development of a versatile shuttle vector for gene expression in Geobacillus spp.,” Plasmid, vol. 60, no. 1, pp. 45-52, 2008
A. Villalobos, J. E. Ness, C. Gustafsson, J. Minshull, and S. Govindarajan, “Gene Designer: A synthetic biology tool for constructing artificial DNA segments,” BMC Bioinformatics, vol. 7, pp. 285-292, 2006
X. Yang, Q. Yuan, Y. Zheng, H. Ma, T. Chen, and X. Zhao, “An engineered non-oxidative glycolysis pathway for acetone production in Escherichia coli” Biotechnol Lett, vol. 38, no. 8, pp. 1359-1365, 2016.
J. Zhou, H. Zhang, Y. Zhang, Y. Li, and Y. Ma, “Designing and creating a modularized synthetic pathway in cyanobacterium Synechocystis enables production of acetone from carbon dioxide,” Metab Eng, vol. 14, no. 4, pp. 394-400, 2012
J. Zhou, K. Wu, and C. V Rao, “Evolutionary engineering of Geobacillus thermoglucosidasius for improved ethanol production,” Biotechnol Bioeng, vol. 9999, pp. 1-12, 2016

### Sequence Overview

Sequence ID NO:

Amino acid sequences

| | | |
|---|---|---|
| 1 | GHH_c20420 | acetyl-CoA acetyltransferase (EC 2.3.1.9) |
| | | from Geobacillus sp. GHH01, NCBI accession |
| | | number: WP_015375064 |
| 2 | Slip_0499 | acetyl-CoA acetyltransferase (EC 2.3.1.9) |
| | | from Syntrophothermus lipocalidus DSM |
| | | 12680, NCBI accession number: |
| | | WP_013174685 |
| 3 | Caur_1461 | acetyl-CoA acetyltransferase (EC 2.3.1.9) |
| | | from Chloroflexus aurantiacus J-10-fl, NCBI |
| | | accession number: WP_012257345 |
| 4 | Slip_0479 | acetyl-CoA acetyltransferase (EC 2.3.1.9) |
| | | from Syntrophothermus lipocalidus DSM |
| | | 12680, NCBI accession number: |
| | | WP_013174665 |
| 5 | Tfu_1520 | acetyl-CoA acetyltransferase (EC 2.3.1.9) |
| | | from Thermobifida fusca YX, NCBI accession |
| | | number: WP_011291951 |
| 6 | Tfu_0436 | acetyl-CoA acetyltransferase (EC 2.3.1.9) |
| | | from Thermobifida fusca YX, NCBI accession |
| | | number: WP_011290883 |
| 7 | Slip_0880 | acetyl-CoA acetyltransferase (EC 2.3.1.9) |
| | | from Syntrophothermus lipocalidus DSM |
| | | 12680, NCBI accession number: |
| | | WP_013175062 |
| 8 | Tfu_2394 | acetyl-CoA acetyltransferase (EC 2.3.1.9) |
| | | from Thermobifida fusca YX, NCBI accession |
| | | number: WP_011292817 |
| 9 | Slip_1236 | acetyl-CoA acetyltransferase (EC 2.3.1.9) |
| | | from Syntrophothermus lipocalidus DSM |
| | | 12680, NCBI accession number: |
| | | WP_013175411 |
| 10 | Caur_1540 | acetyl-CoA acetyltransferase (EC 2.3.1.9) |
| | | from Chloroflexus aurantiacus J-10-fl, NCBI |
| | | accession number: WP_012257413 |
| 11 | Tfu_0253 | acetyl-CoA acetyltransferase (EC 2.3.1.9) |
| | | from Thermobifida fusca YX, NCBI accession |
| | | number: WP_011290700 |
| 12 | SVA_3859 | 3-oxoacyl-ACP synthase (EC 2.3.3.20) from |
| | | Sulfurifustis variabilis, NCBI accession |
| | | number: BAU50393 |
| 13 | Despr_2661 | acyl-CoA:acyl-CoA alkyltransferase (EC |
| | | 2.3.3.20) from Desulfobulbus propionicus |
| | | DSM 2032, NCBI accession number: |
| | | WP_015725323 |
| 14 | CHY_1604 | acetyl-CoA acetyltransferase (EC 2.3.1.9) |
| | | from Carboxydothermus hydrogenoformans Z- |
| | | 2901, NCBI accession number: |
| | | WP_011344507 |
| 15 | CHY_1288 | acetyl-CoA acetyltransferase (EC 2.3.1.9) |
| | | from Carboxydothermus hydrogenoformans Z- |
| | | 2901, NCBI accession number: |
| | | WP_011344198 |
| 16 | Slip_2085 | acetyl-CoA acetyltransferase (EC 2.3.1.9) |
| | | from Syntrophothermus lipocalidus DSM |
| | | 12680, NCBI accession number: |
| | | WP_013176234 |
| 17 | Slip_0465 | acetyl-CoA acetyltransferase (EC 2.3.1.9) |
| | | from Syntrophothermus lipocalidus DSM |
| | | 12680, NCBI accession number: |
| | | WP_013174651 |
| 18 | CHY_1355 | acetyl-CoA acetyltransferase (EC 2.3.1.9) |
| | | from Carboxydothermus hydrogenoformans Z- |
| | | 2901, NCBI accession number: |
| | | WP_011344263 |
| 19 | Tle2 subunit A | acetyl CoA transferase subunit A (EC 2.8.3.8) |
| | | from Pseudothermotoga lettingae TMO, NCBI |
| | | accession number: WP_012003587 |
| 20 | Tle2 subunit B | acetyl CoA transferase subunit B (EC 2.8.3.9) |
| | | from Pseudothermotoga lettingae TMO, NCBI |
| | | accession number: WP_012003586 |
| 21 | Dde2 | 3-oxoacid CoA transferase (EC 2.8.3.5) from |
| | | Deferribacter desulfuricans SSM1, NCBI |
| | | accession number: WP_013008615 |
| 22 | Ghh2 subunit A | 3-oxoacid CoA transferase (EC 2.8.3.5) |
| | | subunit A from Geobacillus sp. GHH01, NCBI |
| | | accession number: WP_015375068 |
| 23 | Ghh2 subunit B | 3-oxoacid CoA transferase (EC 2.8.3.5) |
| | | subunit B from Geobacillus sp. GHH01, NCBI |
| | | accession number: WP_041470504 |
| 24 | Tme subunit A | 3-oxoacid CoA transferase (EC 2.8.3.5) |
| | | subunit A from Thermosipho melanesiensis, |
| | | NCBI accession number: WP_012057350 |
| 25 | Tme subunit B | 3-oxoacid CoA transferase (EC 2.8.3.5) |
| | | subunit B from Thermosipho melanesiensis |
| | | BI429, NCBI accession number: |
| | | WP_012057349 |
| 26 | Pth | propionate CoA-transferase/acyl |
| | | CoA:acetate/3-ketoacid CoA-transferase (EC |
| | | 2.8.3.1) from Pelotomaculum |
| | | thermopropionicum SI, NCBI accession |
| | | number: WP_012031684 |
| 27 | Rma | acyl-CoA thioesterase II (EC 3.1.2 .—) from |
| | | Rhodothermus marinus DSM 4252, NCBI |
| | | accession number: WP_012844627 |
| 28 | Cac | acetoacetate decarboxylase (EC 4.1.1.4), |
| | | from Clostridium acetobutylicum ATCC |
| | | 824, NCBI accession number: WP_010890849 |
| 29 | Tbr | isopropanol dehydrogenase (EC 1.1.1.80) |
| | | from Thermoanaerobacter brockii Ako-1, NCBI |
| | | accession number: WP_041589967 |
| 59 | Dde1 | acetyl-CoA acetyltransferase (EC 2.3.1.9) |
| | | from Deferribacter desulfuricans SSM1, NCBI |
| | | accession number: WP_013008537 |
| 60 | Rxy2 | acetyl-CoA acetyltransferase (EC 2.3.1.9) |
| | | from Rubrobacter xylanophilus, NCBI |
| | | accession number: WP_011565330 |

DNA sequences

| | | |
|---|---|---|
| 30 | GHH_c20420 | NCBI accession number: GHH_RS10045 |
| 31 | Slip_0499 | NCBI accession number: SLIP_RS02420 |
| 32 | Caur_1461 | NCBI accession number: CAUR_RS07625, |
| | | codon-optimised sequence |
| 33 | Slip_0479 | NCBI accession number: SLIP_RS02325 |
| 34 | Tfu_1520 | NCBI accession number: TFU_RS07525 |
| 35 | Tfu_0436 | NCBI accession number: TFU_RS02175 |
| 36 | Slip_0880 | NCBI accession number: SLIP_RS04340 |
| 37 | Tfu_2394 | NCBI accession number: TFU_RS11895 |
| 38 | Slip_1236 | NCBI accession number: SLIP_RS06120 |
| 39 | Caur_1540 | NCBI accession number: CAUR_RS08085 |
| 40 | Tfu_0253 | NCBI accession number: TFU_RS01260 |
| 41 | SVA_3859 | NCBI accession number: SVA_3859 |
| 42 | Despr_2661 | NCBI accession number: DESPR_RS13350 |
| 43 | CHY_1604 | NCBI accession number: CHY_RS07495 |
| 44 | CHY_1288 | NCBI accession number: CHY_RS05980 |
| 45 | Slip_2085 | NCBI accession number: SLIP_RS10325 |
| 46 | Slip_0465 | NCBI accession number: SLIP_RS02255 |
| 47 | CHY_1355 | NCBI accession number: CHY_RS06295 |
| 48 | Tle2 subunit A | NCBI accession number: TLET_RS07995 |
| | | (codon optimized) |
| 49 | Tle2 subunit B | NCBI accession number: TLET_RS07990 |
| | | (codon optimized) |
| 50 | Dde2 | NCBI accession number: DEFDS_RS09685 |
| | | (codon optimized) |
| 51 | Ghh2 subunit A | NCBI accession number: GHH_RS10065 |
| 52 | Ghh2 subunit B | NCBI accession number: GHH_RS10060 |
| 53 | Tme subunit A | NCBI accession number: TMEL_RS05880 |
| | | (codon optimized) |
| 54 | Tme subunit B | NCBI accession number: TMEL_RS05885 |
| | | (codon optimized) |
| 55 | Pth | NCBI accession number: PTH_RS02645 |
| | | (codon optimized) |
| 56 | Rma | NCBI accession number: RMAR_RS10655 |
| | | (codon optimized) |
| 57 | Cac | NCBI accession number: CA_RS20035 |
| 58 | Tbr | NCBI accession number: THEBR_RS01110 |
| | | (codon optimized) |
| 61 | Dde1 | NCBI accession number: WP_013008537 |
| 62 | Rxy2 | NCBI accession number: WP_011565330 |
| 63 | Caur_1461 | NCBI accession number: CAUR_RS07625, |

### Items

1. A method of producing one or more compounds selected from acetone, butanone and isopropanol, said method comprising the steps of:
   a) Providing a thermophilic cell, preferably a thermophilic bacterial or a thermophilic archaeal cell expressing:
      i) a first enzyme selected from:
         an acetyl-CoA acetyltransferase (EC 2.3.1.9) selected from GHH_c20420 as set forth in SEQ ID NO: 1, Slip_0499 as set forth in SEQ ID NO: 2, Caur_1461 as set forth in SEQ ID NO: 3, Slip_0479 as set forth in SEQ ID NO: 4, Tfu_1520 as set forth in SEQ ID NO: 5, Tfu_0436 as set forth in SEQ ID NO: 6, Slip_0880 as set forth in SEQ ID NO: 7, Tfu_2394 as set forth in SEQ ID NO: 8, Slip_1236 as set forth in SEQ ID NO: 9, Caur 1540 as set forth in SEQ ID NO: 10, Tfu_0253 as set forth in SEQ ID NO: 11, CHY_1604 as set forth in SEQ ID NO: 14, CHY_1288 as set forth in SEQ ID NO: 15, Slip_2085 as set forth in SEQ ID NO: 16, Slip_0465 as set forth in SEQ ID NO: 17, Dde1 as set forth in SEQ ID NO: 59, Rxy2 as set forth in SEQ ID NO: 60 and CHY_1355 as set forth in SEQ ID NO: 18,
         an enzyme of EC number 2.3.3.20 selected from the 3-oxoacyl-ACP synthase SVA_3859 as set forth in SEQ ID NO: 12 and the acyl-CoA:acyl-CoA alkyltransferase Despr_2661 as set forth in SEQ ID NO: 13; and
         functional variants thereof having at least 70% homology, similarity or identity thereto;
      ii) a second enzyme selected from an acetate CoA transferase, a 3-oxoacid CoA transferase, an acyl CoA:acetate/3-ketoacid CoA-transferase, and an acyl-CoA thioesterase II,
         wherein the second enzyme is selected from: Tle2, Dde2 (EC 2.8.3.5) as set forth in SEQ ID NO: 21, Ghh2 (EC 2.8.3.5), Tme (EC 2.8.3.8), Pth (EC 2.8.3.1) as set forth in SEQ ID NO: 26 and Rma (EC 3.1.2.-) as set forth in SEQ ID NO: 27, or functional variants thereof having at least 70% homology, similarity or identity thereto, wherein Tle2 consists of Tle2 subunit A (EC 2.8.3.8) as set forth in SEQ ID NO: 19 and Tle2 subunit B (EC 2.8.3.9) as set forth in SEQ ID NO: 20, wherein Ghh2 consists of Ghh2 subunit A as set forth in SEQ ID NO: 22 and Ghh2 subunit B as set forth in SEQ ID NO: 23, and wherein Tme consists of Tme subunit A as set forth in SEQ ID NO: 24 and Tme subunit B as set forth in SEQ ID NO: 25, or functional variants thereof having at least 70% homology, similarity or identity thereto,
         and
      iii) an acetoacetate decarboxylase (EC 4.1.1.4), preferably Cac as set forth in SEQ ID NO: 28 or a functional variant thereof having at least 70% homology, similarity or identity thereto;
      iv) optionally an isopropanol dehydrogenase (EC 1.1.1.80), preferably Tbr as set forth in SEQ ID NO: 29 or a functional variant thereof having at least 70% homology, similarity or identity thereto;
   b) cultivating the bacterial cell in a bioreactor comprising a cultivation broth at a temperature of between 42 and 80° C., such as between 50 and 75° C., for example at 60° C., thereby producing the one or more compounds;
   c) recovering the one or more compounds produced during step b).
2. The method according to item 1, wherein the thermophilic cell has an optimal growth temperature of between 42 and 80° C., such as between 50 and 75° C., for example at 60° C.
3. The method according to any one of the preceding items, wherein the thermophilic cell is of a genus selected from: Geobacillus, Thermoanaerobacterium, Thermoanaerobacter, Caldanaerobacter, Bacillus, Thermoclostridium, Anoxybacillus, Caldicellulosiruptor, Moorella, Thermus, Thermotoga, Pseudothermotoga, Chloroflexus, Anaerocellum, Rhodothermus, Sulfolobus, Thermococcus, Pyrococcus and Clostridium.
4. The method according to any one of the preceding items, wherein the thermophilic cell is of a species selected from: Geobacillus thermoglucosidasius, Geobacillus toebii, Geobacillus stearothermophilus, Geobacillus thermodenitrificans, Geobacillus kaustophilus, Geobacillus thermoleovorans, Geobacillus thermocatenulatus, Thermoanaerobacterium xylanolyticum, Thermoanaerobacterium saccharotyticum, Thermoanaerobacterium thermosaccharolyticum, Thermoanaerobacter mathranii, Thermoanaerobacter pseudoethanolicus, Thermoanaerobacter brockii, Thermoanaerobacter kivui, Thermoanaerobacter brockii, Caldanaerobacter subterraneus, Clostridium thermocellum, Clostridium thermosuccinogenes, Thermoclostridium stercorarium, Bacillus subtilis, Bacillus licheniformis, Bacillus coagulans, Bacillus smithii, Bacillus methanolicus, Bacillus flavothermus, Anoxybacillus kamchatkensis, Anoxybacillus gonensis, Caldicellulosiruptor bescii, Caldicellulosiruptor saccharolyticus, Caldicellulosiruptor kristjanssonii, Caldicellulosiruptor owensensis, Caldicellulosiruptor lactoaceticus, Moorella thermoacetica, Moorella thermoautotrophica, Thermus thermophilus, Thermus aquaticus, Thermotoga maritima, Pseudothermotoga lettingae, Pseudothermotoga thermarum, Chloroflexus aurantiacus, Anaerocellum thermophilum, Rhodothermus marinus, Sulfolobus acidocaldarius, Sulfolobus islandicus, Sulfolobus solfataricus, Thermococcus barophilus, Thermococcus kodakarensis, Pyrococcus abyssi, Pyrococcus furiosus, preferably the cell is a Geobacillus thermoglucosidasius cell, a Bacillus subtilis cell or a Clostridium thermocellum cell.
5. The method according to any one of the preceding items, wherein the cultivation broth comprises a fermentable substrate comprising a carbon source such as a carbohydrate, for example glucose, xylose, or a mixture thereof, or such as a biomass hydrolysate.
6. The method according to any one of the preceding items, wherein the thermophilic cell is an acetogenic cell, and wherein the cell is provided with carbon monoxide, carbon dioxide, hydrogen or a mixture thereof.
7. The method according to any one of the preceding items, wherein the one or more compounds comprises acetone and optionally isopropanol, wherein the cell is capable of synthesising acetyl-CoA and/or wherein the cultivation broth comprises acetic acid or acetate.
8. The method according to any one of the preceding items, wherein acetone is produced with a titer of at least 0.8 g/L, such as at least 0.9 g/L, such as at least 1.0 g/L, such as at least 1.1 g/L, such as at least 1.2 g/L, such as at least 1.3 g/L, such as at least 1.4 g/L, such as at least 1.5 g/L, such as at least 1.6 g/L, such as at least 1.7 g/L, such as at least 1.8 g/L, such as at least 1.9 g/L, such as at least 2.0 g/L, such as at least 5 g/L, such as at least 7.5 g/L, such as at least 10 g/L, such as at least 12.5 g/L, such as at least 15 g/L, such as at least 20 g/L, such as at least 25 g/L, such as at least 50 g/L, such as at least 75 g/L, such as at least 100 g/L, such as at least 150 g/L, such as at least 250 g/L, or more.
9. The method according to any one of the preceding items, wherein at least acetone is produced and wherein the first enzyme is CHY_1288 as set forth in SEQ ID NO: 15, CHY_1355 as set forth in SEQ ID NO: 18, Caur_1540 as set forth in SEQ ID NO: 10, GHH_c20420 as set forth in SEQ ID NO: 1, Caur_1461 as set forth in SEQ ID NO: 3, Dde1 as set forth in SEQ ID NO: 59, Rxy2 as set forth in SEQ ID NO: 60 or Slip_0880 as set forth in SEQ ID NO: 7, or a functional variant thereof having at least 70% homology, similarity or identity thereto, preferably Caur_1461 as set forth in SEQ ID NO: 3, Rxy2 as set forth in SEQ ID NO: 60, Slip_0880 as set forth in SEQ ID NO: 7 or Dde1 as set forth in SEQ ID NO: 59.
10. The method according to any one of the preceding items, wherein the thermophilic cell expresses Tbr as set forth in SEQ ID NO: 29 or a functional variant thereof having at least 70% homology, similarity or identity thereto, whereby at least part of the acetone produced is converted to isopropanol.
11. The method according to any one of the preceding items, wherein at least isopropanol is produced with a titer of at least 0.05 g/L, such as at least 0.075 g/L, such as at least 0.1 g/L, such as at least 0.2 g/L, such as at least 0.3 g/L, such as at least 0.4 g/L, such as at least 0.5 g/L, such as at least 0.75 g/L, such as at least 1.0 g/L, such as at least 2.0 g/L, such as at least 3.0 g/L, such as at least 4.0 g/L, such as at least 5.0 g/L, such as at least 7.5 g/L, such as at least 10.0 g/L or more, such as at least 12.5 g/L, such as at least 15 g/L, such as at least 20 g/L, such as at least 25 g/L, such as at least 50 g/L, such as at least 75 g/L, such as at least 100 g/L, such as at least 150 g/L, such as at least 250 g/L, or more.
12. The method according to any one of the preceding items, wherein the one or more compounds comprises butanone, wherein the cultivation broth comprises propionic acid or propionate.
13. The method according to any one of the preceding items, wherein at least butanone is produced, and wherein the first enzyme is GHH_c20420 as set forth in SEQ ID NO: 1, Slip_0499 as set forth in SEQ ID NO: 2, Caur_1461 as set forth in SEQ ID NO: 3, Slip_0479 as set forth in SEQ ID NO: 4, Dde1 as set forth in SEQ ID NO: 59, Rxy2 as set forth in SEQ ID NO: 60, Tfu_1520 as set forth in SEQ ID NO: 5, or Tfu_0436 as set forth in SEQ ID NO: 6, or a functional variant thereof having at least 70% homology, similarity or identity thereto, preferably GHH_c20420 as set forth in SEQ ID NO: 1, Slip_0499 as set forth in SEQ ID NO: 2, Caur_1461 as set forth in SEQ ID NO: 3, Dde1 as set forth in SEQ ID NO: 59, Rxy2 as set forth in SEQ ID NO: 60 or Slip_0479 as set forth in SEQ ID NO: 4.
14. The method according to any one of the preceding items, wherein butanone is produced with a titer of at least 0.05 g/L, such as at least 0.075 g/L, such as at least 0.1 g/L, such as at least 0.2 g/L, such as at least 0.3 g/L, such as at least 0.4 g/L, such as at least 0.5 g/L, such as at least 0.75 g/L, such as at least 1.0 g/L, such as at least 2.0 g/L, such as at least 3.0 g/L, such as at least 4.0 g/L, such as at least 5.0 g/L, such as at least 7.5 g/L, such as at least 10.0 g/L or more, such as at least 12.5 g/L, such as at least 15 g/L, such as at least 20 g/L, such as at least 25 g/L, such as at least 50 g/L, such as at least 75 g/L, such as at least 100 g/L, such as at least 150 g/L, such as at least 250 g/L, or more.
15. The method according to any one of the preceding items, wherein the second enzyme is:
   i) Tle2 or a functional variant thereof, wherein Tle2 consists of Tle2 subunit A (EC 2.8.3.8) as set forth in SEQ ID NO: 19 and Tle2 subunit B (EC 2.8.3.9) as set forth in SEQ ID NO: 20, and wherein the functional variant of Tle2 consists of a subunit having at least 70% homology, similarity or identity to Tle2 subunit A (EC 2.8.3.8) as set forth in SEQ ID NO: 19 and another subunit having at least 70% homology, similarity or identity to Tle2 subunit B (EC 2.8.3.9) as set forth in SEQ ID NO: 20;
   ii) Dde2 as set forth in SEQ ID NO: 21 or a functional variant thereof having at least 70% homology, similarity or identity thereto; or
   iii) Ghh2 or a functional variant thereof, wherein Ghh2 consists of Ghh2 subunit A as set forth in SEQ ID NO: 22 and Ghh2 subunit B as set forth in SEQ ID NO: 23 and wherein the functional variant of Ghh2 consists of a subunit having at least 70% homology, similarity or identity to Ghh2 subunit A as set forth in SEQ ID NO: 22 and another subunit having at least 70% homology, similarity or identity to Ghh2 subunit B as set forth in SEQ ID NO: 23.
16. The method according to any one of the preceding items, wherein the acetoacetate decarboxylase is Cac as set forth in SEQ ID NO: 28 or a functional variant thereof having at least 70% homology, similarity or identity thereto.
17. The method according to any one of the preceding items, wherein the cultivation in step b) is a continuous fermentation.
18. The method according to any one of the preceding items, wherein step c) comprises recovering the one or more volatile compounds from the off-gas produced during step b), such as by condensation.
19. The method according to any one of the preceding items, wherein the first enzyme consists of an acetyl-CoA acetyltransferase (EC 2.3.1.9) selected from GHH_c20420 as set forth in SEQ ID NO: 1, Slip_0499 as set forth in SEQ ID NO: 2, Caur_1461 as set forth in SEQ ID NO: 3, Slip_0479 as set forth in SEQ ID NO: 4, Slip_0880 as set forth in SEQ ID NO: 7, and Dde1 as set forth in SEQ ID NO: 59, or functional variants thereof having at least 70% identity or similarity thereto having acetyl-CoA acetyltransferase activity.
20. The method according to any one of the preceding items, wherein the second enzyme is selected from: Tle2 and Dde2 (EC 2.8.3.5) as set forth in SEQ ID NO: 21 or functional variants thereof having at least 70% identity or similarity thereto having acetate CoA transferase, 3-oxoacid CoA transferase, acyl CoA:acetate/3-ketoacid CoA-transferase, or acyl-CoA thioesterase II activity.
21. The method according to any one of the preceding items, wherein at least acetone is produced and wherein the first enzyme is Caur_1461 (SEQ ID NO: 3), Slip_0880 (SEQ ID NO: 7) or Dde1 (SEQ ID NO: 59) or a functional variant thereof having at least 70% identity or similarity thereto having acetyl-CoA acetyltransferase activity.
22. The method according to any one of the preceding items, wherein the one or more compounds comprises butanone, wherein the cultivation broth comprises propionic acid or propionate, and/or wherein the first enzyme is GHH_c20420 as set forth in SEQ ID NO: 1, Slip_0499 as set forth in SEQ ID NO: 2, Caur_1461 as set forth in SEQ ID NO: 3, Dde1 as set forth in SEQ ID NO: 59, or Slip_0479 as set forth in SEQ ID NO: 4, or a functional variant thereof having at least 70% identity or similarity thereto having acetyl-CoA acetyltransferase activity.
23. The method according to any one of the preceding items, wherein the thermophilic cell expresses Cac as set forth in SEQ ID NO: 28 or a functional variant thereof having at least 70% identity or similarity thereto having acetoacetate decarboxylase activity, wherein the thermophilic cell further expresses:
   i) Dde1 as set forth in SEQ ID NO: 59 and Dde2 as set forth in SEQ ID NO: 21; or functional variants thereof having at least 70% identity or similarity thereto having acetyl-CoA acetyltransferase activity or acetate CoA transferase, 3-oxoacid CoA transferase, acyl CoA:acetate/3-ketoacid CoA-transferase, or acyl-CoA thioesterase II activity, respectively; whereby at least acetone is produced; or
   ii) Caur_1461 as set forth in SEQ ID NO: 3 and Tle2; or functional variants thereof having at least 70% identity or similarity thereto having acetyl-CoA acetyltransferase activity or acetate CoA transferase, 3-oxoacid CoA transferase, acyl CoA:acetate/3-ketoacid CoA-transferase, or acyl-CoA thioesterase II activity, respectively; whereby at least acetone and/or butanone is produced; or
   iii) Slip_0880 as set forth in SEQ ID NO: 7 and Tle2; or functional variants thereof having at least 70% identity or similarity thereto having acetyl-CoA acetyltransferase activity or acetate CoA transferase, 3-oxoacid CoA transferase, acyl CoA:acetate/3-ketoacid CoA-transferase, or acyl-CoA thioesterase II activity, respectively; whereby at least acetone is produced; or
   iv) GHH_c20420 as set forth in SEQ ID NO: 1 and Tle2; or functional variants thereof having at least 70% identity or similarity thereto having acetyl-CoA acetyltransferase activity or acetate CoA transferase, 3-oxoacid CoA transferase, acyl CoA:acetate/3-ketoacid CoA-transferase, or acyl-CoA thioesterase II activity, respectively; whereby at least butanone is produced; or
   v) Slip_0499 as set forth in SEQ ID NO: 2 and Tle2; or functional variants thereof having at least 70% identity or similarity thereto having acetyl-CoA acetyltransferase activity or acetate CoA transferase, 3-oxoacid CoA transferase, acyl CoA:acetate/3-ketoacid CoA-transferase, or acyl-CoA thioesterase II activity, respectively; whereby at least butanone is produced; or
   vi) Slip_0479 as set forth in SEQ ID NO: 4 and Tle2; or functional variants thereof having at least 70% identity or similarity thereto having acetyl-CoA acetyltransferase activity or acetate CoA transferase, 3-oxoacid CoA transferase, acyl CoA:acetate/3-ketoacid CoA-transferase, or acyl-CoA thioesterase II activity, respectively; whereby at least butanone is produced;
      wherein Tle2 consists of Tle2 subunit A (EC 2.8.3.8) as set forth in SEQ ID NO: 19 and Tle2 subunit B (EC 2.8.3.9) as set forth in SEQ ID NO: 20 or functional variants thereof having at least 70% identity or similarity thereto having acetate CoA transferase, 3-oxoacid CoA transferase, acyl CoA:acetate/3-ketoacid CoA-transferase, or acyl-CoA thioesterase II activity.
24. A thermophilic cell capable of producing acetone and/or butanone and optionally isopropanol, said cell being a bacterial cell or an archaeal cell and expressing:
   i) a first enzyme selected from:
      an acetyl-CoA acetyltransferase (EC 2.3.1.9) selected from GHH_c20420 as set forth in SEQ ID NO: 1, Slip_0499 as set forth in SEQ ID NO: 2, Caur_1461 as set forth in SEQ ID NO: 3, Slip_0479 as set forth in SEQ ID NO: 4, Tfu_1520 as set forth in SEQ ID NO: 5, Tfu_0436 as set forth in SEQ ID NO: 6, Slip_0880 as set forth in SEQ ID NO: 7, Tfu_2394 as set forth in SEQ ID NO: 8, Slip_1236 as set forth in SEQ ID NO: 9, Caur_1540 as set forth in SEQ ID NO: 10, Tfu_0253 as set forth in SEQ ID NO: 11, CHY_1604 as set forth in SEQ ID NO: 14, CHY_1288 as set forth in SEQ ID NO: 15, Slip_2085 as set forth in SEQ ID NO: 16, Slip_0465 as set forth in SEQ ID NO: 17, Dde1 as set forth in SEQ ID NO: 59, Rxy2 as set forth in SEQ ID NO: 60 and CHY_1355 as set forth in SEQ ID NO: 18, or
      an enzyme of EC number 2.3.3.20 selected from the 3-oxoacyl-ACP synthase SVA_3859 as set forth in SEQ ID NO: 12 and the acyl-CoA:acyl-CoA alkyltransferase Despr_2661 as set forth in SEQ ID NO: 13; and
      functional variants thereof having at least 70% homology, similarity or identity thereto;
   ii) a second enzyme selected from an acetate CoA transferase, a 3-oxoacid CoA transferase, an acyl CoA:acetate/3-ketoacid CoA-transferase, and an acyl-CoA thioesterase II
      wherein the second enzyme is selected from: Tle2, Dde2 (EC 2.8.3.5) as set forth in SEQ ID NO: 21, Ghh2 (EC 2.8.3.5), Tme (EC 2.8.3.8), Pth (EC 2.8.3.1) as set forth in SEQ ID NO: 26 and Rma (EC 3.1.2.-) as set forth in SEQ ID NO: 27, or functional variants thereof having at least 70% homology, similarity or identity thereto, wherein Tle2 consists of Tle2 subunit A (EC 2.8.3.8) as set forth in SEQ ID NO: 19 and Tle2 subunit B (EC 2.8.3.9) as set forth in SEQ ID NO: 20, wherein Ghh2 consists of Ghh2 subunit A as set forth in SEQ ID NO: 22 and Ghh2 subunit B as set forth in SEQ ID NO: 23, and wherein Tme consists of Tme subunit A as set forth in SEQ ID NO: 24 and Tme subunit B as set forth in SEQ ID NO: 25, and
   iii) an acetoacetate decarboxylase (EC 4.1.1.4), preferably Cac as set forth in SEQ ID NO: 28 or a functional variant thereof having at least 70% homology, similarity or identity thereto;
whereby the cell can convert acetyl-CoA to acetone, thereby producing acetone with a titer of at least 0.8 g/L;
and/or whereby the cell can convert acetyl-CoA and propionyl-CoA to butanone, thereby producing butanone;
   iv) optionally an isopropanol dehydrogenase (EC 1.1.1.80), preferably Tbr as set forth in SEQ ID NO: 29 or a functional variant thereof having at least 70% homology, similarity or identity thereto,
      whereby the cell can further convert acetone to isopropanol, thereby producing isopropanol.
25. The thermophilic cell according to item 24, said cell being of a genus selected from: Geobacillus, Thermoanaerobacterium, Thermoanaerobacter, Caldanaerobacter, Bacillus, Thermoclostridium, Anoxybacillus, Caldicellulosiruptor, Moorella, Thermus, Thermotoga, Pseudothermotoga, Chloroflexus, Anaerocellum, Rhodothermus, Sulfolobus, Thermococcus, Pyrococcus and Clostridium.
26. The thermophilic cell according to item 25, said cell being of a species selected from: Geobacillus thermoglucosidasius, Geobacillus toebii, Geobacillus stearothermophilus, Geobacillus thermodenitrificans, Geobacillus kaustophilus, Geobacillus thermoleovorans, Geobacillus thermocatenulatus, Thermoanaerobacterium xylanolyticum, Thermoanaerobacterium saccharotyticum, Thermoanaerobacterium thermosaccharolyticum, Thermoanaerobacter mathranii, Thermoanaerobacter pseudoethanolicus, Thermoanaerobacter brockii, Thermoanaerobacter kivui Thermoanaerobacter brockii, Caldanaerobacter subterraneus, Clostridium thermocellum, Clostridium thermosuccinogenes, Thermoclostridium stercorarium, Bacillus subtilis, Bacillus licheniformis, Bacillus coagulans, Bacillus smithii, Bacillus methanolicus, Bacillus flavothermus, Anoxybacillus kamchatkensis, Anoxybacillus gonensis, Caldicellulosiruptor bescii, Caldicellulosiruptor saccharolyticus, Caldicellulosiruptor kristjanssonii, Caldicellulosiruptor owensensis, Caldicellulosiruptor lactoaceticus, Moorella thermoacetica, Moorella thermoautotrophica, Thermus thermophilus, Thermus aquaticus, Thermotoga maritima, Pseudothermotoga lettingae, Pseudothermotoga thermarum, Chloroflexus aurantiacus, Anaerocellum thermophilum, Rhodothermus marinus, Sulfolobus acidocaldarius, Sulfolobus islandicus, Sulfolobus solfataricus, Thermococcus barophilus, Thermococcus kodakarensis, Pyrococcus abyssi, Pyrococcus furiosus, preferably the cell is a Geobacillus thermoglucosidasius cell, a Bacillus subtilis cell or a Clostridium thermocellum cell.
27. The thermophilic cell according to any one of items 24 to 26, wherein the cell is capable of synthesising acetyl-CoA.
28. The thermophilic cell according to any one of items 24 to 27, wherein the cell comprises one or more polynucleotides encoding the first enzyme, the second enzyme, the acetoacetate decarboxylase and optionally the isopropanol dehydrogenase.
29. The thermophilic cell according to any one of items 24 to 28, wherein the one or more polynucleotides is codon optimised for expression in the cell.
30. The thermophilic cell according to any one of items 24 to 29, wherein the one or more polynucleotides is comprised within a vector or is integrated in the genome of the cell.
31. The thermophilic cell according to any one of items 24 to 30, wherein the one or more polynucleotides is under the control of an inducible promoter or under the control of a constitutive promoter.
32. The thermophilic cell according to any one of items 24 to 31, wherein the cell is a non-natural cell.
33. The thermophilic cell according to any one of items 24 to 32, wherein the first enzyme consists of an acetyl-CoA acetyltransferase (EC 2.3.1.9) selected from GHH_c20420 as set forth in SEQ ID NO: 1, Slip_0499 as set forth in SEQ ID NO: 2, Caur_1461 as set forth in SEQ ID NO: 3, Slip_0479 as set forth in SEQ ID NO: 4, Slip_0880 as set forth in SEQ ID NO: 7, and Dde1 as set forth in SEQ ID NO: 59, or functional variants thereof having at least 70% identity or similarity thereto having acetyl-CoA acetyltransferase activity.
34. The thermophilic cell according to any one of items 24 to 33, wherein the second enzyme is selected from: Tle2 and Dde2 (EC 2.8.3.5) as set forth in SEQ ID NO: 21 or functional variants thereof having at least 70% identity or similarity thereto having acetate CoA transferase, 3-oxoacid CoA transferase, acyl CoA:acetate/3-ketoacid CoA-transferase, or acyl-CoA thioesterase II activity.
35. The thermophilic cell according to any one of items 24 to 34, wherein the first enzyme is Caur_1461 (SEQ ID NO: 3), Slip_0880 (SEQ ID NO: 7) or Dde1 (SEQ ID NO: 59) or a functional variant thereof having at least 70% identity or similarity thereto having acetyl-CoA acetyltransferase activity.
36. The thermophilic cell according to any one of items 24 to 35, wherein the first enzyme is GHH_c20420 as set forth in SEQ ID NO: 1, Slip_0499 as set forth in SEQ ID NO: 2, Caur_1461 as set forth in SEQ ID NO: 3, Dde1 as set forth in SEQ ID NO: 59, or Slip 0479 as set forth in SEQ ID NO: 4, or a functional variant thereof having at least 70% identity or similarity thereto having acetyl-CoA acetyltransferase activity.
37. The thermophilic cell according to any one of items 24 to 36, wherein the thermophilic cell expresses Cac as set forth in SEQ ID NO: 28 or a functional variant thereof having at least 70% identity or similarity thereto having acetoacetate decarboxylase activity, wherein the thermophilic cell further expresses:
   i) Dde1 as set forth in SEQ ID NO: 59 and Dde2 as set forth in SEQ ID NO: 21; or functional variants thereof having at least 70% identity or similarity thereto having acetyl-CoA acetyltransferase activity or acetate CoA transferase, 3-oxoacid CoA transferase, acyl CoA:acetate/3-ketoacid CoA-transferase, or acyl-CoA thioesterase II activity, respectively; whereby at least acetone is produced; or
   ii) Caur_1461 as set forth in SEQ ID NO: 3 and Tle2; or functional variants thereof having at least 70% identity or similarity thereto having acetyl-CoA acetyltransferase activity or acetate CoA transferase, 3-oxoacid CoA transferase, acyl CoA:acetate/3-ketoacid CoA-transferase, or acyl-CoA thioesterase II activity, respectively; whereby at least acetone and/or butanone is produced; or
   iii) Slip_0880 as set forth in SEQ ID NO: 7 and Tle2; or functional variants thereof having at least 70% identity or similarity thereto having acetyl-CoA acetyltransferase activity or acetate CoA transferase, 3-oxoacid CoA transferase, acyl CoA:acetate/3-ketoacid CoA-transferase, or acyl-CoA thioesterase II activity, respectively; whereby at least acetone is produced; or
   iv) GHH_c20420 as set forth in SEQ ID NO: 1 and Tle2; or functional variants thereof having at least 70% identity or similarity thereto having acetyl-CoA acetyltransferase activity or acetate CoA transferase, 3-oxoacid CoA transferase, acyl CoA:acetate/3-ketoacid CoA-transferase, or acyl-CoA thioesterase II activity, respectively; whereby at least butanone is produced; or
   v) Slip_0499 as set forth in SEQ ID NO: 2 and Tle2; or functional variants thereof having at least 70% identity or similarity thereto having acetyl-CoA acetyltransferase activity or acetate CoA transferase, 3-oxoacid CoA transferase, acyl CoA:acetate/3-ketoacid CoA-transferase, or acyl-CoA thioesterase II activity, respectively; whereby at least butanone is produced; or
   vi) Slip_0479 as set forth in SEQ ID NO: 4 and Tle2; or functional variants thereof having at least 70% identity or similarity thereto having acetyl-CoA acetyltransferase activity or acetate CoA transferase, 3-oxoacid CoA transferase, acyl CoA:acetate/3-ketoacid CoA-transferase, or acyl-CoA thioesterase II activity, respectively; whereby at least butanone is produced;
wherein Tle2 consists of Tle2 subunit A (EC 2.8.3.8) as set forth in SEQ ID NO: 19 and Tle2 subunit B (EC 2.8.3.9) as set forth in SEQ ID NO: 20 or functional variants thereof having at least 70% identity or similarity thereto having acetate CoA transferase, 3-oxoacid CoA transferase, acyl CoA:acetate/3-ketoacid CoA-transferase, or acyl-CoA thioesterase II activity.
38. A nucleic acid construct for modifying a thermophilic cell selected from a thermophilic bacterial cell and a thermophilic archaeal cell, comprising:
   i) a polynucleotide encoding an acetyl-CoA acetyltransferase (EC 2.3.1.9) or a functional variant thereof having at least 70% homology, similarity or identity thereto, wherein the acetyl-CoA acetyltransferase is selected from GHH_c20420 as set forth in SEQ ID NO: 1, Slip_0499 as set forth in SEQ ID NO: 2, Caur_1461 as set forth in SEQ ID NO: 3, Slip_0479 as set forth in SEQ ID NO: 4, Tfu_1520 as set forth in SEQ ID NO: 5, Tfu_0436 as set forth in SEQ ID NO: 6, Slip_0880 as set forth in SEQ ID NO: 7, Tfu_2394 as set forth in SEQ ID NO: 8, Slip_1236 as set forth in SEQ ID NO: 9, Caur_1540 as set forth in SEQ ID NO: 10, Tfu_0253 as set forth in SEQ ID NO: 11, CHY_1604 as set forth in SEQ ID NO: 14, CHY_1288 as set forth in SEQ ID NO: 15, Slip_2085 as set forth in SEQ ID NO: 16, Slip_0465 as set forth in SEQ ID NO: 17, Dde1 as set forth in SEQ ID NO: 59, Rxy2 as set forth in SEQ ID NO: 60 and CHY_1355 as set forth in SEQ ID NO: 18, and/or an enzyme of EC number 2.3.3.20 selected from the 3-oxoacyl-ACP synthase SVA_3859 as set forth in SEQ ID NO: 12 and the acyl-CoA:acyl-CoA alkyltransferase Despr 2661 as set forth in SEQ ID NO: 13;
   ii) a polynucleotide encoding a second enzyme selected from an acetate CoA transferase, a 3-oxoacid CoA transferase, an acyl CoA:acetate/3-ketoacid CoA-transferase, and an acyl-CoA thioesterase II
      wherein the second enzyme is selected from: Tle2, Dde2 (EC 2.8.3.5) as set forth in SEQ ID NO: 21, Ghh2 (EC 2.8.3.5), Tme (EC 2.8.3.8), Pth (EC 2.8.3.1) as set forth in SEQ ID NO: 26 and Rma (EC 3.1.2.-) as set forth in SEQ ID NO: 27, or functional variants thereof having at least 70% homology, similarity or identity thereto, wherein Tle2 consists of Tle2 subunit A (EC 2.8.3.8) as set forth in SEQ ID NO: 19 and Tle2 subunit B (EC 2.8.3.9) as set forth in SEQ ID NO: 20, wherein Ghh2 consists of Ghh2 subunit A as set forth in SEQ ID NO: 22 and Ghh2 subunit B as set forth in SEQ ID NO: 23, and wherein Tme consists of Tme subunit A as set forth in SEQ ID NO: 24 and Tme subunit B as set forth in SEQ ID NO: 25, or functional variants thereof having at least 70% homology, similarity or identity thereto, and
   iii) a polynucleotide encoding an acetoacetate decarboxylase (EC 4.1.1.4) or a functional variant thereof having at least 70% homology, similarity or identity thereto, preferably Cac as set forth in SEQ ID NO: 28.
39. The nucleic acid construct according to item 38, further comprising a polynucleotide encoding an isopropanol dehydrogenase (EC 1.1.1.80), preferably Tbr as set forth in SEQ ID NO: 29 or a functional variant thereof having at least 70% homology, similarity or identity thereto, preferably wherein the polynucleotide encoding the isopropanol dehydrogenase or a functional variant thereof having at least 70% identity or similarity thereto having isopropanol dehydrogenase activity is SEQ ID NO: 58 or a homologue thereof having at least 70% identity thereto.
40. The nucleic acid construct according to any one of items 38 to 39, wherein one or more of the polynucleotides is codon-optimised for expression in said thermophilic cell.
41. The nucleic acid construct according to any one of items 38 to 40, wherein one or more of the polynucleotides is under the control of an inducible promoter or of a constitutive promoter.
42. The nucleic acid construct according to any one of items 38 to 41, wherein the thermophilic cell is of a genus selected from: Geobacillus, Thermoanaerobacterium, Thermoanaerobacter, Caldanaerobacter, Bacillus, Thermoclostridium, Anoxybacillus, Caldicellulosiruptor, Moorella, Thermus, Thermotoga, Pseudothermotoga, Chloroflexus, Anaerocellum, Rhodothermus, Sulfolobus, Thermococcus, Pyrococcus and Clostridium.
43. The nucleic acid construct according to any one of items 38 to 42, wherein the thermophilic cell is of a species selected from: Geobacillus thermoglucosidasius, Geobacillus toebii, Geobacillus stearothermophilus, Geobacillus thermodenitrificans, Geobacillus kaustophilus, Geobacillus thermoleovorans, Geobacillus thermocatenulatus, Thermoanaerobacterium xylanolyticum, Thermoanaerobacterium saccharotyticum, Thermoanaerobacterium thermosaccharolyticum, Thermoanaerobacter mathranii, Thermoanaerobacter pseudoethanolicus, Thermoanaerobacter brockii, Thermoanaerobacter kivui, Thermoanaerobacter brockii, Caldanaerobacter subterraneus, Clostridium thermocellum, Clostridium thermosuccinogenes, Thermoclostridium stercorarium, Bacillus subtilis, Bacillus licheniformis, Bacillus coagulans, Bacillus smithii, Bacillus methanolicus, Bacillus flavothermus, Anoxybacillus kamchatkensis, Anoxybacillus gonensis, Caldicellulosiruptor bescii, Caldicellulosiruptor saccharolyticus, Caldicellulosiruptor kristjanssonii, Caldicellulosiruptor owensensis, Caldicellulosiruptor lactoaceticus, Moorella thermoacetica, Moorella thermoautotrophica, Thermus thermophilus, Thermus aquaticus, Thermotoga maritima, Pseudothermotoga lettingae, Pseudothermotoga thermarum, Chloroflexus aurantiacus, Anaerocellum thermophilum, Rhodothermus marinus, Sulfolobus acidocaldarius, Sulfolobus islandicus, Sulfolobus solfataricus, Thermococcus barophilus, Thermococcus kodakarensis, Pyrococcus abyssi, Pyrococcus furiosus, preferably the cell is a Geobacillus thermoglucosidasius cell, a Bacillus subtilis cell or a Clostridium thermocellum cell.
44. The nucleic acid construct according to any one of items 38 to 43, wherein the acetyl-CoA acetyltransferase is selected from CHY_1288 as set forth in SEQ ID NO: 15, CHY_1355 as set forth in SEQ ID NO: 18, Caur_1540 as set forth in SEQ ID NO: 10, GHH_c20420 as set forth in SEQ ID NO: 1, Caur_1461 as set forth in SEQ ID NO: 3, Dde1 as set forth in SEQ ID NO: 59, Rxy2 as set forth in SEQ ID NO: 60 and Slip_0880 as set forth in SEQ ID NO: 7, or functional variants thereof having at least 70% homology, similarity or identity thereto.
45. The nucleic acid construct according to any one of items 38 to 44, wherein the isopropanol dehydrogenase (EC 1.1.1.80) is Tbr as set forth in SEQ ID NO: 29 or a functional variant thereof having at least 70% homology, similarity or identity thereto.
46. The nucleic acid construct according to any one of items 38 to 45, wherein the acetyl-CoA acetyltransferase is GHH_c20420 as set forth in SEQ ID NO: 1, Slip_0499 as set forth in SEQ ID NO: 2, Caur_1461 as set forth in SEQ ID NO: 3, Slip_0479 as set forth in SEQ ID NO: 4, Dde1 as set forth in SEQ ID NO: 59, Rxy2 as set forth in SEQ ID NO: 60, Tfu_1520 as set forth in SEQ ID NO: 5, or Tfu_0436 as set forth in SEQ ID NO: 6, or a functional variant thereof having at least 70% homology, similarity or identity thereto, preferably GHH_c20420 as set forth in SEQ ID NO: 1, Slip_0499 as set forth in SEQ ID NO: 2, Caur_1461 as set forth in SEQ ID NO: 3, Dde1 as set forth in SEQ ID NO: 59, Rxy2 as set forth in SEQ ID NO: 60, or Slip_0479 as set forth in SEQ ID NO: 4.
47. The nucleic acid construct according to any one of items 38 to 46, wherein the second enzyme is:
   i) Tle2 or a functional variant thereof, wherein Tle2 consists of Tle2 subunit A (EC 2.8.3.8) as set forth in SEQ ID NO: 19 and Tle2 subunit B (EC 2.8.3.9) as set forth in SEQ ID NO: 20, and wherein the functional variant of Tle2 consists of a subunit having at least 70% homology, similarity or identity to Tle2 subunit A (EC 2.8.3.8) as set forth in SEQ ID NO: 19 and another subunit having at least 70% homology, similarity or identity to Tle2 subunit B (EC 2.8.3.9) as set forth in SEQ ID NO: 20;
   ii) Dde2 as set forth in SEQ ID NO: 21 or a functional variant thereof having at least 70% homology, similarity or identity thereto; or
   iii) Ghh2 or a functional variant thereof, wherein Ghh2 consists of Ghh2 subunit A as set forth in SEQ ID NO: 22 and Ghh2 subunit B as set forth in SEQ ID NO: 23 and wherein the functional variant of Ghh2 consists of a subunit having at least 70% homology, similarity or identity to Ghh2 subunit A as set forth in SEQ ID NO: 22 and another subunit having at least 70% homology, similarity or identity to Ghh2 subunit B as set forth in SEQ ID NO: 23.
48. The nucleic acid construct according to any one of items 38 to 47, wherein the acetoacetate decarboxylase is Cac as set forth in SEQ ID NO: 28 or a functional variant thereof having at least 70% homology, similarity or identity thereto.
49. The nucleic acid construct according to any one of items 38 to 48, wherein:
   i) the acetyl-CoA acetyltransferase is selected from GHH_c20420 as set forth in SEQ ID NO: 1, Slip_0499 as set forth in SEQ ID NO: 2, Caur_1461 as set forth in SEQ ID NO: 3, Slip_0479 as set forth in SEQ ID NO: 4, Slip_0880 as set forth in SEQ ID NO: 7, and Dde1 as set forth in SEQ ID NO: 59, or functional variants thereof having at least 70% identity or similarity thereto having acetyl-CoA acetyltransferase activity; and
   ii) the second enzyme is selected from: Tle2 and Dde2 as set forth in SEQ ID NO: 21, or functional variants thereof having at least 70% identity or similarity thereto having at least 70% identity or similarity thereto having acetate CoA transferase, 3-oxoacid CoA transferase, acyl CoA:acetate activity.
50. The nucleic acid construct according to any one of items 38 to 49, wherein:
   i) the polynucleotide encoding the acetyl-CoA acetyltransferase (EC 2.3.1.9) or the functional variant thereof having at least 70% identity or similarity thereto having acetyl-CoA acetyltransferase activity is selected from SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO:43, SEQ ID NO: 44, SEQ ID NO: 46, SEQ ID NO: 61, SEQ ID NO: 62 and SEQ ID NO: 47 or homologues thereof having at least 70% identity thereto; and
   ii) the polynucleotide encoding the second enzyme is selected from: SEQ ID NO: 48 and SEQ ID NO: 49; SEQ ID NO: 50; SEQ ID NO: 51 and SEQ ID NO: 52, SEQ ID NO: 53 and SEQ ID NO: 54; and SEQ ID NO: 56, or homologues thereof having at least 70% identity thereto; and
   iii) the polynucleotide encoding the acetoacetate decarboxylase or the functional variant thereof having at least 70% homology, similarity or identity thereto is SEQ ID NO: 57, or a homologue thereof having at least 70% identity thereto.
51. The nucleic acid construct according to any one of items 38 to 50, wherein the polynucleotide encoding an acetyl-CoA acetyltransferase (EC 2.3.1.9) or a functional variant thereof having at least 70% identity or similarity thereto having acetyl-CoA acetyltransferase activity is selected from SEQ ID NO: 30, SEQ ID NO: 31, SEQ ID NO: 32, SEQ ID NO: 33, SEQ ID NO: 36, and SEQ ID NO: 61, or homologues thereof having at least 70% identity thereto;
   and/or
   wherein the polynucleotide encoding the second enzyme is selected from:
   i) SEQ ID NO: 48 and SEQ ID NO: 49, or homologues thereof having at least 70% identity thereto; and
   ii) SEQ ID NO: 50 or a homologue thereof having at least 70% identity thereto;
      and/or
   wherein the polynucleotide encoding the acetoacetate decarboxylase or a functional variant thereof having at least 70% identity or similarity thereto having acetoacetate decarboxylase activity is SEQ ID NO: 57 or a homologue thereof having at least 70% identity thereto.
52. The nucleic acid construct according to any one of items 38 to 51, comprising SEQ ID NO: 57 or a homologue thereof having at least 70% identity thereto and further comprising:
   i) SEQ ID NO: 61 and SEQ ID NO: 50; or
   ii) SEQ ID NO: 32; and SEQ ID NO: 48 and SEQ ID NO: 49; or
   iii) SEQ ID NO: 36; and SEQ ID NO: 48 and SEQ ID NO: 49; or
   iv) SEQ ID NO: 30; and SEQ ID NO: 48 and SEQ ID NO: 49; or
   v) SEQ ID NO: 31; and SEQ ID NO: 48 and SEQ ID NO: 49; or
   vi) SEQ ID NO: 33; and SEQ ID NO: 48 and SEQ ID NO: 49;
   or homologues thereof having at least 70% identity thereto;
   preferably wherein the nucleic acid construct comprises SEQ ID NO: 57 and ii) or iii).
53. A vector comprising the nucleic acid construct according to any one of items 38 to 52.
54. A thermophilic cell comprising the nucleic acid construct according to any one of items 38 to 52 and/or the vector according to item 53, wherein the thermophilic cell is a thermophilic bacterial cell or a thermophilic archaeal cell.
55. A kit comprising the nucleic acid construct according to any one of items 38 to 52, the vector according to item 53 or the thermophilic cell according to item 54.

1. A method of producing one or more compounds selected from acetone, butanone and isopropanol, said method comprising the steps of:
a) Providing a thermophilic cell, preferably a thermophilic bacterial or a thermophilic archaeal cell expressing:
i) a first enzyme consisting of an acetyl-CoA acetyltransferase (EC 2.3.1.9) selected from Slip_0880 as set forth in SEQ ID NO: 7, Caur_1461 as set forth in SEQ ID NO: 3, GHH_c20420 as set forth in SEQ ID NO: 1, Slip_0499 as set forth in SEQ ID NO: 2, Slip_0479 as set forth in SEQ ID NO: 4, and Dde1 as set forth in SEQ ID NO: 59,
or functional variants thereof having at least 70% identity or similarity thereto having acetyl-CoA acetyltransferase activity;
ii) a second enzyme selected from an acetate CoA transferase, a 3-oxoacid CoA transferase, an acyl CoA:acetate/3-ketoacid CoA-transferase, and an acyl-CoA thioesterase 1l,
wherein the second enzyme is selected from: Tle2 and Dde2 (EC 2.8.3.5) as set forth in SEQ ID NO: 21 or functional variants thereof having at least 70% identity or similarity thereto having acetate CoA transferase, 3-oxoacid CoA transferase, acyl CoA:acetate/3-ketoacid CoA-transferase, or acyl-CoA thioesterase II activity,
wherein Tle2 consists of Tle2 subunit A (EC 2.8.3.8) as set forth in SEQ ID NO: 19 and Tle2 subunit B (EC 2.8.3.9) as set forth in SEQ ID NO: 20 or functional variants thereof having at least 70% identity or similarity thereto having acetate CoA transferase, 3-oxoacid CoA transferase, acyl CoA:acetate/3-ketoacid CoA-transferase, or acyl-CoA thioesterase II activity,
and
iii) an acetoacetate decarboxylase (EC 4.1.1.4), wherein the acetoacetate decarboxylase is Cac as set forth in SEQ ID NO: 28 or a functional variant thereof having at least 70% identity or similarity thereto having acetoacetate decarboxylase activity; and
iv) optionally an isopropanol dehydrogenase (EC 1.1.1.80), wherein the isopropanol dehydrogenase is Tbr as set forth in SEQ ID NO: 29 or a functional variant thereof having at least 70% identity or similarity thereto having isopropanol dehydrogenase activity;
b) cultivating the thermophilic cell in a bioreactor comprising a cultivation broth at a temperature of between 42 and 80° C., such as between 50 and 75° C., for example at 60° C., thereby producing the one or more compounds;
c) recovering the one or more compounds produced during step b).

2. The method according to claim 1, wherein the thermophilic cell has an optimal growth temperature of between 42 and 80° C., such as between 50 and 75° C., for example at 60° C.

3. The method according to any one of the preceding claims, wherein the thermophilic cell is of a genus selected from: Geobacillus, Thermoanaerobacterium, Thermoanaerobacter, Caldanaerobacter, Bacillus, Thermoclostridium, Anoxybacillus, Caldicellulosiruptor, Moorella, Thermus, Thermotoga, Pseudothermotoga, Chloroflexus, Anaerocellum, Rhodothermus, Sulfolobus, Thermococcus, Pyrococcus and Clostridium, preferably wherein the thermophilic cell is of a species selected from: Geobacillus thermoglucosidasius, Geobacillus toebii, Geobacillus stearothermophilus, Geobacillus thermodenitrificans, Geobacillus kaustophilus, Geobacillus thermoleovorans, Geobacillus thermocatenulatus, Thermoanaerobacterium xylanolyticum, Thermoanaerobacterium saccharotyticum, Thermoanaerobacterium thermosaccharolyticum, Thermoanaerobacter mathranii, Thermoanaerobacter pseudoethanolicus, Thermoanaerobacter brockii, Thermoanaerobacter kivui, Thermoanaerobacter brockii, Caldanaerobacter subterraneus, Clostridium thermocellum, Clostridium thermosuccinogenes, Thermoclostridium stercorarium, Bacillus subtilis, Bacillus licheniformis, Bacillus coagulans, Bacillus smithii, Bacillus methanolicus, Bacillus flavothermus, Anoxybacillus kamchatkensis, Anoxybacillus gonensis, Caldicellulosiruptor bescii, Caldicellulosiruptor saccharolyticus, Caldicellulosiruptor kristjanssonii, Caldicellulosiruptor owensensis, Caldicellulosiruptor lactoaceticus, Moorella thermoacetica, Moorella thermoautotrophica, Thermus thermophilus, Thermus aquaticus, Thermotoga maritima, Pseudothermotoga lettingae, Pseudothermotoga thermarum, Chloroflexus aurantiacus, Anaerocellum thermophilum, Rhodothermus marinus, Sulfolobus acidocaldarius, Sulfolobus islandicus, Sulfolobus solfataricus, Thermococcus barophilus, Thermococcus kodakarensis, Pyrococcus abyssi, Pyrococcus furiosus, preferably the cell is a Geobacillus thermoglucosidasius cell, a Bacillus subtilis cell or a Clostridium thermocellum cell.

4. The method according to any one of the preceding claims, wherein the cultivation broth comprises a fermentable substrate comprising a carbon source such as a carbohydrate, for example glucose, xylose, or a mixture thereof, or such as a biomass hydrolysate.

5. The method according to any one of the preceding claims, wherein the one or more compounds comprises acetone and optionally isopropanol, wherein the cell is capable of synthesising acetyl-CoA and/or wherein the cultivation broth comprises acetic acid or acetate, and/or wherein acetone is produced with a titer of at least 0.8 g/L, such as at least 0.9 g/L, such as at least 1.0 g/L, such as at least 1.1 g/L, such as at least 1.2 g/L, such as at least 1.3 g/L, such as at least 1.4 g/L, such as at least 1.5 g/L, such as at least 1.6 g/L, such as at least 1.7 g/L, such as at least 1.8 g/L, such as at least 1.9 g/L, such as at least 2.0 g/L, such as at least 5 g/L, such as at least 7.5 g/L, such as at least 10 g/L, such as at least 12.5 g/L, such as at least 15 g/L, such as at least 20 g/L, such as at least 25 g/L, such as at least 50 g/L, such as at least 75 g/L, such as at least 100 g/L, such as at least 150 g/L, such as at least 250 g/L, or more.

6. The method according to any one of the preceding claims, wherein at least acetone is produced and wherein the first enzyme is Caur_1461 (SEQ ID NO: 3), Slip_0880 (SEQ ID NO: 7) or Dde1 (SEQ ID NO: 59) or a functional variant thereof having at least 70% identity or similarity thereto having acetyl-CoA acetyltransferase activity.

7. The method according to any one of the preceding claims, wherein the thermophilic cell expresses Tbr as set forth in SEQ ID NO: 29 or a functional variant thereof having at least 70% identity or similarity thereto having isopropanol dehydrogenase activity, whereby at least part of the acetone produced is converted to isopropanol, preferably wherein at least isopropanol is produced with a titer of at least 0.05 g/L, such as at least 0.075 g/L, such as at least 0.1 g/L, such as at least 0.2 g/L, such as at least 0.3 g/L, such as at least 0.4 g/L, such as at least 0.5 g/L, such as at least 0.75 g/L, such as at least 1.0 g/L, such as at least 2.0 g/L, such as at least 3.0 g/L, such as at least 4.0 g/L, such as at least 5.0 g/L, such as at least 7.5 g/L, such as at least 10.0 g/L or more, such as at least 12.5 g/L, such as at least 15 g/L, such as at least 20 g/L, such as at least 25 g/L, such as at least 50 g/L, such as at least 75 g/L, such as at least 100 g/L, such as at least 150 g/L, such as at least 250 g/L, or more.

8. The method according to any one of the preceding claims, wherein the one or more compounds comprises butanone, wherein the cultivation broth comprises propionic acid or propionate, and/or
wherein the first enzyme is Caur_1461 as set forth in SEQ ID NO: 3, GHH_c20420 as set forth in SEQ ID NO: 1, Slip_0499 as set forth in SEQ ID NO: 2, Dde1 as set forth in SEQ ID NO: 59, or Slip_0479 as set forth in SEQ ID NO: 4, or a functional variant thereof having at least 70% identity or similarity thereto having acetyl-CoA acetyltransferase activity, and/or
wherein butanone is produced with a titer of at least 0.05 g/L, such as at least 0.075 g/L, such as at least 0.1 g/L, such as at least 0.2 g/L, such as at least 0.3 g/L, such as at least 0.4 g/L, such as at least 0.5 g/L, such as at least 0.75 g/L, such as at least 1.0 g/L, such as at least 2.0 g/L, such as at least 3.0 g/L, such as at least 4.0 g/L, such as at least 5.0 g/L, such as at least 7.5 g/L, such as at least 10.0 g/L or more, such as at least 12.5 g/L, such as at least 15 g/L, such as at least 20 g/L, such as at least 25 g/L, such as at least 50 g/L, such as at least 75 g/L, such as at least 100 g/L, such as at least 150 g/L, such as at least 250 g/L, or more.

9. The method according to any one of the preceding claims, wherein the thermophilic cell expresses Cac as set forth in SEQ ID NO: 28 or a functional variant thereof having at least 70% identity or similarity thereto having acetoacetate decarboxylase activity, wherein the thermophilic cell further expresses:
i) Slip_0880 as set forth in SEQ ID NO: 7 and Tle2; or functional variants thereof having at least 70% identity or similarity thereto having acetyl-CoA acetyltransferase activity or acetate CoA transferase, 3-oxoacid CoA transferase, acyl CoA:acetate/3-ketoacid CoA-transferase, or acyl-CoA thioesterase II activity, respectively; whereby at least acetone is produced; or
ii) Caur_1461 as set forth in SEQ ID NO: 3 and Tle2; or functional variants thereof having at least 70% identity or similarity thereto having acetyl-CoA acetyltransferase activity or acetate CoA transferase, 3-oxoacid CoA transferase, acyl CoA:acetate/3-ketoacid CoA-transferase, or acyl-CoA thioesterase II activity, respectively; whereby at least acetone and/or butanone is produced; or
iii) GHH_c20420 as set forth in SEQ ID NO: 1 and Tle2; or functional variants thereof having at least 70% identity or similarity thereto having acetyl-CoA acetyltransferase activity or acetate CoA transferase, 3-oxoacid CoA transferase, acyl CoA:acetate/3-ketoacid CoA-transferase, or acyl-CoA thioesterase II activity, respectively; whereby at least butanone is produced; or
iv) Dde1 as set forth in SEQ ID NO: 59 and Dde2 as set forth in SEQ ID NO: 21; or functional variants thereof having at least 70% identity or similarity thereto having acetyl-CoA acetyltransferase activity or acetate CoA transferase, 3-oxoacid CoA transferase, acyl CoA:acetate/3-ketoacid CoA-transferase, or acyl-CoA thioesterase II activity, respectively; whereby at least acetone is produced; or;
v) Slip_0499 as set forth in SEQ ID NO: 2 and Tle2; or functional variants thereof having at least 70% identity or similarity thereto having acetyl-CoA acetyltransferase activity or acetate CoA transferase, 3-oxoacid CoA transferase, acyl CoA:acetate/3-ketoacid CoA-transferase, or acyl-CoA thioesterase II activity, respectively; whereby at least butanone is produced; or
vi) Slip_0479 as set forth in SEQ ID NO: 4 and Tle2; or functional variants thereof having at least 70% identity or similarity thereto having acetyl-CoA acetyltransferase activity or acetate CoA transferase, 3-oxoacid CoA transferase, acyl CoA:acetate/3-ketoacid CoA-transferase, or acyl-CoA thioesterase II activity, respectively; whereby at least butanone is produced;
wherein Tle2 consists of Tle2 subunit A (EC 2.8.3.8) as set forth in SEQ ID NO: 19 and Tle2 subunit B (EC 2.8.3.9) as set forth in SEQ ID NO: 20 or functional variants thereof having at least 70% identity or similarity thereto having acetate CoA transferase, 3-oxoacid CoA transferase, acyl CoA:acetate/3-ketoacid CoA-transferase, or acyl-CoA thioesterase II activity,
preferably wherein the thermophilic cell expresses Cac as set forth in SEQ ID NO: 28 or a functional variant thereof having at least 70% identity or similarity thereto having acetoacetate decarboxylase activity, and further expresses i) or ii).

10. A thermophilic cell capable of producing acetone and/or butanone and optionally isopropanol, said cell being a bacterial cell or an archaeal cell and expressing:
i) a first enzyme selected consisting of
an acetyl-CoA acetyltransferase (EC 2.3.1.9) selected from Slip_0880 as set forth in SEQ ID NO: 7, Caur_1461 as set forth in SEQ ID NO: 3, GHH_c20420 as set forth in SEQ ID NO: 1, Slip_0499 as set forth in SEQ ID NO: 2, Slip_0479 as set forth in SEQ ID NO: 4, and Dde1 as set forth in SEQ ID NO: 59, or functional variants thereof having at least 70% identity or similarity thereto having acetyl-CoA acetyltransferase activity;
ii) a second enzyme selected from an acetate CoA transferase, a 3-oxoacid CoA transferase, an acyl CoA:acetate/3-ketoacid CoA-transferase, and an acyl-CoA thioesterase II
wherein the second enzyme is selected from: Tle2 and Dde2 (EC 2.8.3.5) as set forth in SEQ ID NO: 21, or functional variants thereof having at least 70% identity or similarity thereto having acetate CoA transferase, 3-oxoacid CoA transferase, acyl CoA:acetate/3-ketoacid CoA-transferase, or acyl-CoA thioesterase II activity,
wherein Tle2 consists of Tle2 subunit A (EC 2.8.3.8) as set forth in SEQ ID NO: 19 and Tle2 subunit B (EC 2.8.3.9) as set forth in SEQ ID NO: 20 or functional variants thereof having at least 70% identity or similarity thereto having acetate CoA transferase, 3-oxoacid CoA transferase, acyl CoA:acetate/3-ketoacid CoA-transferase, or acyl-CoA thioesterase II activity, and
iii) an acetoacetate decarboxylase (EC 4.1.1.4), wherein the acetoacetate decarboxylase is Cac as set forth in SEQ ID NO: 28 or a functional variant thereof having at least 70% identity or similarity thereto having acetoacetate decarboxylase activity;
whereby the cell can convert acetyl-CoA to acetone, thereby producing acetone with a titer of at least 0.8 g/L;
and/or whereby the cell can convert acetyl-CoA and propionyl-CoA to butanone, thereby producing butanone; and
iv) optionally an isopropanol dehydrogenase (EC 1.1.1.80), wherein the isopropanol dehydrogenase is Tbr as set forth in SEQ ID NO: 29 or a functional variant thereof having at least 70% identity or similarity thereto having isopropanol dehydrogenase activity,
whereby the cell can further convert acetone to isopropanol, thereby producing isopropanol,
preferably wherein the thermophilic cell is according to any one of the preceding claims.

11. The thermophilic cell according to claim 10, wherein the thermophilic cell expresses Cac as set forth in SEQ ID NO: 28 or a functional variant thereof having at least 70% identity or similarity thereto having acetoacetate decarboxylase activity, wherein the thermophilic cell further expresses:
i) Slip_0880 as set forth in SEQ ID NO: 7 and Tle2; or functional variants thereof having at least 70% identity or similarity thereto having acetyl-CoA acetyltransferase activity or acetate CoA transferase, 3-oxoacid CoA transferase, acyl CoA:acetate/3-ketoacid CoA-transferase, or acyl-CoA thioesterase II activity, respectively; or
ii) Caur_1461 as set forth in SEQ ID NO: 3 and Tle2; or functional variants thereof having at least 70% identity or similarity thereto having acetyl-CoA acetyltransferase activity or acetate CoA transferase, 3-oxoacid CoA transferase, acyl CoA:acetate/3-ketoacid CoA-transferase, or acyl-CoA thioesterase II activity, respectively; or
iii) GHH_c20420 as set forth in SEQ ID NO: 1 and Tle2; or functional variants thereof having at least 70% identity or similarity thereto having acetyl-CoA acetyltransferase activity or acetate CoA transferase, 3-oxoacid CoA transferase, acyl CoA:acetate/3-ketoacid CoA-transferase, or acyl-CoA thioesterase II activity, respectively; or
iv) Dde1 as set forth in SEQ ID NO: 59 and Dde2 as set forth in SEQ ID NO: 21; or functional variants thereof having at least 70% identity or similarity thereto having acetyl-CoA acetyltransferase activity or acetate CoA transferase, 3-oxoacid CoA transferase, acyl CoA:acetate/3-ketoacid CoA-transferase, or acyl-CoA thioesterase II activity, respectively; or
v) Slip_0499 as set forth in SEQ ID NO: 2 and Tle2; or functional variants thereof having at least 70% identity or similarity thereto having acetyl-CoA acetyltransferase activity or acetate CoA transferase, 3-oxoacid CoA transferase, acyl CoA:acetate/3-ketoacid CoA-transferase, or acyl-CoA thioesterase II activity, respectively; or
vi) Slip_0479 as set forth in SEQ ID NO: 4 and Tle2; or functional variants thereof having at least 70% identity or similarity thereto having acetyl-CoA acetyltransferase activity or acetate CoA transferase, 3-oxoacid CoA transferase, acyl CoA:acetate/3-ketoacid CoA-transferase, or acyl-CoA thioesterase II activity, respectively;
wherein Tle2 consists of Tle2 subunit A (EC 2.8.3.8) as set forth in SEQ ID NO: 19 and Tle2 subunit B (EC 2.8.3.9) as set forth in SEQ ID NO: 20 or functional variants thereof having at least 70% identity or similarity thereto having acetate CoA transferase, 3-oxoacid CoA transferase, acyl CoA:acetate/3-ketoacid CoA-transferase, or acyl-CoA thioesterase II activity.

12. The thermophilic cell according to claim 11, wherein the thermophilic cell expresses i) or ii).

13. The thermophilic cell according to any one of claims 10 to 12, wherein the thermophilic cell further expresses Tbr as set forth in SEQ ID NO: 29 or a functional variant thereof having at least 70% identity or similarity thereto having isopropanol dehydrogenase activity.

14. A nucleic acid construct for modifying a thermophilic cell selected from a thermophilic bacterial cell and a thermophilic archaeal cell, comprising:
i) a polynucleotide encoding an acetyl-CoA acetyltransferase (EC 2.3.1.9) or a functional variant thereof having at least 70% identity or similarity thereto having acetyl-CoA acetyltransferase activity, wherein the acetyl-CoA acetyltransferase is selected from Slip_0880 as set forth in SEQ ID NO: 7, Caur_1461 as set forth in SEQ ID NO: 3, GHH_c20420 as set forth in SEQ ID NO: 1, Slip_0499 as set forth in SEQ ID NO: 2, Slip_0479 as set forth in SEQ ID NO: 4, and Dde1 as set forth in SEQ ID NO: 59;
ii) a polynucleotide encoding a second enzyme selected from an acetate CoA transferase, a 3-oxoacid CoA transferase, an acyl CoA:acetate/3-ketoacid CoA-transferase, and an acyl-CoA thioesterase II
wherein the second enzyme is selected from: Tle2 and Dde2 (EC 2.8.3.5) as set forth in SEQ ID NO: 21, or functional variants thereof having at least 70% identity or similarity thereto having at least 70% identity or similarity thereto having acetate CoA transferase, 3-oxoacid CoA transferase, acyl CoA:acetate/3-ketoacid CoA-transferase, or acyl-CoA thioesterase II activity,
wherein Tle2 consists of Tle2 subunit A (EC 2.8.3.8) as set forth in SEQ ID NO: 19 and Tle2 subunit B (EC 2.8.3.9) as set forth in SEQ ID NO: 20, or functional variants thereof having at least 70% identity or similarity thereto having at least 70% identity or similarity thereto having acetate CoA transferase, 3-oxoacid CoA transferase, acyl CoA:acetate/3-ketoacid CoA-transferase, or acyl-CoA thioesterase II activity, and
iii) a polynucleotide encoding an acetoacetate decarboxylase (EC 4.1.1.4) or a functional variant thereof having at least 70% identity or similarity thereto having acetoacetate decarboxylase activity, wherein the acetoacetate decarboxylase is Cac as set forth in SEQ ID NO: 28, and
iv) optionally a polynucleotide encoding an isopropanol dehydrogenase (EC 1.1.1.80), wherein the isopropanol dehydrogenase is Tbr as set forth in SEQ ID NO: 29 or a functional variant thereof having at least 70% identity or similarity thereto having isopropanol dehydrogenase activity.

15. The nucleic acid construct according to claim 14,
wherein the polynucleotide encoding an acetyl-CoA acetyltransferase (EC 2.3.1.9) or a functional variant thereof having at least 70% identity or similarity thereto having acetyl-CoA acetyltransferase activity is selected from SEQ ID NO: 30, SEQ ID NO: 31, SEQ ID NO: 32, SEQ ID NO: 33, SEQ ID NO: 36, and SEQ ID NO: 61, or homologues thereof having at least 70% identity thereto; and/or
wherein the polynucleotide encoding the second enzyme is selected from:
iii) SEQ ID NO: 48 and SEQ ID NO: 49, or homologues thereof having at least 70% identity thereto; and
iv) SEQ ID NO: 50 or a homologue thereof having at least 70% identity thereto;
and/or
wherein the polynucleotide encoding the acetoacetate decarboxylase or a functional variant thereof having at least 70% identity or similarity thereto having acetoacetate decarboxylase activity is SEQ ID NO: 57 or a homologue thereof having at least 70% identity thereto.

16. The nucleic acid construct according to any one of claims 14 to 15, wherein the polynucleotide encoding the isopropanol dehydrogenase or a functional variant thereof having at least 70% identity or similarity thereto having isopropanol dehydrogenase activity is SEQ ID NO: 58 or a homologue thereof having at least 70% identity thereto.

17. A vector comprising the nucleic acid construct according to any one of claims 14 to 16.

18. A thermophilic cell comprising the nucleic acid construct according to any one of claims 14 to 16 and/or the vector according to claim 17, wherein the thermophilic cell is a thermophilic bacterial cell or a thermophilic archaeal cell.


SEQUENCE LISTING (63 sequences)

SEQ ID NO: 1 - protein, 391 aa, Geobacillus sp. GHH01
MREVVITAAVRTPIGTFGGVFKDLLPTDLIVPVLEEAVKRSQIEKDEVNEVILGHCIQRTDIPNTARTAALLAGFPHTTTGFTIQRQCASGMQAVISAAMQIQVGLSDVVIAGGVESMSSSPYILKQHRWGARLQHQQVRDSVWEVLEDPIHHVMMGETAENLAERYGITREEQDELALLSHRRAILAMESGYFDSQIVPITVKTRKEEIVVTKDEHPRADVTKEKLASLRPVFRKNGTVTAGNASGINDGAAALVLMSAEYAQQRGIEPLAKVVGYSVAGVDPLVMGRGPVPAVQKGLERVNWTLAEADLIEINEAFAAQYLAVERELRLDRDKVNVNGSGISLGHPIGCTGARIVVSLIHELQRRQLEKGIASLCVGGGMGTAVFIEAL

SEQ ID NO: 2 - protein, 400 aa, Syntrophothermus lipocalidus DSM 12680
MINEVVMVSACRTAIGDFMGSLKDLKANDLSAITATEALKRAGIQPEMVDSLVLGMCLHHGNDSGPARQVAMAIGMRHSSWACMVNQNCASAMRALEIAANELMLGKSEISLVVGTESMTNVPYILRKARFGYRLFDGDKAEDAMICDGLFDKMVPGHMAITAENVAEKYGITREECDELALLSHTRALKANAEGIFAREIVPVEIKTKKGVKVVDKDEHPMDTSLEKLAQLPPVFKKGGVVTAGNASGINDGSAAAVLMTKKKAEELGIKPLMKLLYVCSEGVDPKFMGLGPAVAIPKVLNKAGLKFEDVEYWEINEAFAAQWLGVGRMLKEDFGIELDLDKVNHNGSGIGLGHPVGCTGLRIQVSMYYEMERLGLTIGGASLCVGGGPAMAALWTRDI

SEQ ID NO: 3 - protein, 395 aa, Chloroflexus aurantiacus J-10-fl
MSEKREVVVLSGVRTAIGTFGGSLKDIPPTELAALVTREAVARSGLQPNEIGHVVFGHVINTEPHDMYLARYAAVRGGLSVETPALTLNRLCGSGLQAIVSAAQYILQGDAEAAVAGGAECMSRGPYSLPAMRFGARMNDSKVVDMMVGALTDPFDDCHMGVTAENVAAKWGISREDQDQLAYESHMRAARAIDEGRFANQIVPVEIKVKGGTAQFMVDEGVRRDTTIDKLAKLRPVFLKDGSVTAGNASSINDAAAAVVLMDRATAERRGYKPLARLVGYSHAAVEPKYMGIGPVPAVRRLLERTGLRISDIDLFEVNEAFAAQALAVIRDLELPPDRTNPNGSGISLGHPIGATGCILTVKAIHELHRTGGRYALVTMCIGGGQGIAAIFERM

SEQ ID NO: 4 - protein, 392 aa, Syntrophothermus lipocalidus DSM 12680
MRDVVIVSGKRTAIGNFLGALKDFSAVDLGTIALKAAINSAGISPDTVEEVAAGHVYQAGCKGNPARQITIGAGCPVETVSVTVNQQCPSAMRALEIISQEIMLGKIDAGAAVGIESMSNVPYLLLKARTGYRMGNGELVDGMLYDALIDAFGNGHQGITAENLAEMYNISREEQDEWAFISHQRACQAIKEGKFKDEIVPVEVKTKKETFLFDTDEHPNPDTTLESLAKLKPAFKKDGTVTAGNASSINDAACAAVVMAHDKAVELGIKPLARIVATASAAVEPRIMGIGVVPAVKRALKFAGMSLDDVQLWEINEAFAAQFLACNRELKLDTEKINVNGSGISLGHPVGCTGLRLVITLINEMKRRNLRYGCAALCAGGGPAMATIIEVL

SEQ ID NO: 5 - protein, 393 aa, Thermobifida fusca YX
MSSPERIIVVDGARTPVGSFGGAFKDVPAHELGAVAARAALQRSGIAASDIDEVVMGCIGQVGPDAYNARRVAIAAGLPESVPAYTVNRLCGSGLQAVWSGAMQIRWGAADIVLAGGDENMSRMPFYDFGARSGYRLGDRTLVDGTVAMLTDPFSNVHMGCTAEAVARKYGVSRAEQDEFALESQRRAAADAARAAFAEEITPVEVGGRKPVVVEVDEHPRPDTTLEGLARLRPVFEKDGTVTAGNASGINDGAAALVLARESVVRERGLKGLAVVESVATAAMDPQLMGYAPVLALRKLFEQTGTSPAVVDVVELNEAFAAQAVAVIRDAGLDPEKTNPYGGAIALGHPVGATGAILTLRVARDLVRRDLELGVVTMCIGGGQALAALLRRV

SEQ ID NO: 6 - protein, 407 aa, Thermobifida fusca YX
MPEAVIVATARSPIGRAFKGSLKDIRPDDLTAQIISAALAKVPQLDPATIDDLLLGCGLPGGEQGFNMARVVAVQLGLDSVPGTTITRYCSSSLQTTRMAFHAIKAGEGDVFISAGVEMVSRFTKGNSDTLPDTKNPLFAEAEARTARRAEGGAEPWRDPREEGKLPDIYIAMGQTAENVAQLRGVSRQRQDEFAVRSQNLAEKALDNGFWEREITPVTLPDGTVVSTDDGPRRGTTYEKVAALDPVFRPDGTVTAGNCCPLNDGAAALIIMSDRKAAELGITPLARIVSTGVSALSPEIMGLGPVEASRQALARANMSIRDIDLVEINEAFAAQVLPSADDLGIDIDSQLNVNGGAIAIGHPFGMTGARITTTLINALQFHDKTFGLETMCVGGGQGMAAIFERLS

SEQ ID NO: 7 - protein, 394 aa, Syntrophothermus lipocalidus DSM 12680
MSREVVLVGACRTPIGTFGGTLKDMTAVQLGTIVMKEALKRAGISGDQVDEVIFGCVLQAGQGQNVARQCAIHAGIPETVTSFTINKVCGSGLRAVSLAAQIIKAGDADIVLAGGTESMTNAPYLVPKARYGYRMGDGKLVDEMVFGGLTDIFNGYHMGITAENVNEMYGITREEQDEFGLRSQERAFAAIESGRFKDEIVPVVIKTKKGEVVFDTDEHPRRTTMEALAKLKPAFKKDGSVTAGNASGINDGAAAVVVMSKEKADELGIKPMARVVSYASGGVDPKIMGVGPVPATKKALAKAGLTLDDIDLIEANEAFAAQSIAVARDMGWDKMMDKVNVNGGAIALGHPIGASGCRILVTLLYEMQKRNAKRGLATLCIGGGQGTTLIVESL

SEQ ID NO: 8 - protein, 395 aa, Thermobifida fusca YX
MPGSVIVGGARTPIGKLLGALSGFAAVDLGAIAIKAALQRAGISGDQVDYVIMGQVLQAGQGQIPSRQASVKAGIPMSVPSLTINKVCLSGLDAIALADQLITAGEFDVVVAGGMESMTNAPHVLPKARHGYKYGSIEVLDATAHDALTDAFDHVSMGLSTERYNARHGMTREEQDAFAARSHQRAAAAIEAGLFKDEIVPVEVPRRKGDPTIVDTDEGVRPDTTVEALARLRPAFDPDGTITAGSSSQISDGACAVVVMSRTKAEELGCEILAEIQAHGNVAGPDNSLHCQPANAIKHALAKAGRDVADLDLVEINEAFASVAIQSMRELGVSEDIVNVNGGAIALGHPVGMSGARIVLHLVHELRRRGGGLGAAGLCGGGGQGDALLLSVPAS

SEQ ID NO: 9 - protein, 393 aa, Syntrophothermus lipocalidus DSM 12680
MAAGIKDKAAVIGMGCTKFGERFDCNLEDLMLEAIEEALADSGLEFNDIDAFWFGTFTSGMAGLAFSNRMKSQYKPVTRIENMCCTGLDAFRNACYAVVSGAYDVVMAIGAEKLKDGGYSGLEVPAEDSDRTMPDLTAPARFAVIAPAYAHKYGLSMQQMKEVMARIAWKNHKNGSLNPKAQFQAEVPIENILKSPMICSPLGIMDCSGVSDGAACAIIVRSEDAKKYRPDPMYVKGIQIAAGPGHSEKHQSYDFTTAWETYYAGQAAYREAGITNPREQIDLAEVHDCFTPTELIIYEDLQFSARGQGWRDALDGFFDLDGKLPVNPDGGLKSFGHPIGASGIRMLYESWLQFHGKAGKRQLENPKIGLAHNLGGQPYQCVVGVAVVGKELG

SEQ ID NO: 10 - protein, 393 aa, Chloroflexus aurantiacus J-10-fl
MDDVVIVGAARTPIGRFNSAYSGLSAIDLGAAAVQAAVQRAGIEADSIDECIMGCVVTAGLGQSPARQAALRAGLPHTIGGLTINKVCGSGLKAVMIGTALIKAGEADVIVAGGMEHMSGAPYLLPQARHGYRLGHGQIIDAVVHDGLWCAFEHHHMGVAAEWIARTFNVTREQQDAYALQSHQRAVAAQDSGAFQAEIAPVTVPGPKGQVNLVTTDEGPRRDTSLAALAKLKPAFVTDGTVTAGNAPGITDGAAAVVLMRASRAAQLGVQPLARIGTAAQAAVKPLELFTAPAFAIERLMKRAGRTLDDYDLFEINEAFAAQVIANLRALALDADRVNVHGGAIALGHPIGASGARVLVTLISALRQRGGQRGIAALCLGGGEAVALEVEVV

SEQ ID NO: 11 - protein, 380 aa, Thermobifida fusca YX
MAEAYIVGAVRTPIGTRKGALAAVHPADLGAHVLKELVNRTGIDPAAVEDVIMGCVTQVGPQALDLARTAWLSAGLPESTPGVTIDRQCGSSQQAVHFAAQGVMSGTQDLVIAAGVENMGMVPMGANVQFAVDNGLSVYGQGWVERYGTQEISQFRGAQLMCEKWGYTREDLEKYALESHRRAAAAIEAGYFDAETAPLAGVTHDEGVRPDTSLEKMAELAPLREGWALTAAVSSQISVGASALLIASERAVAEHGLTPLARIVQLALAGDDPVYMLTAPIPATRIALRKAGLDIDDIDVVEINEAFAPVPMAWIDEIGADPAKVNPNGGAIALGHPLGATGAVLMTKLVHELRRTGGRYGLQTMCEGGGQANVTIIERV

SEQ ID NO: 12 - protein, 342 aa, Sulfurifustis variabilis
MHSVGHSRIISTGMYLPPERLSSRELMEMFRSRERFGLPYEWLERTTGIRERRFAPPDFKSSEMAVAAAREALELGEVSPSQIDAIIYCGVLRDHVEPATAHVVQDKIGARNAIAFDVSNACLGFMNGMHLMDALIATGQARRGLVVTGERGNHYIRKALRVLAELPDNGDFSDLAAALTLGDAGAAAVMGPKLDPETGIKGFVVQSQGQHNGLCVCGDNGEDTHLVTKITEIVRETTRLVGPLYQALMHEHLGWQPSELSRYIPHQVGLRSVRKHAEVAQVPLEIIPITVDYLGNIISATIPVNISLLMKDKKLTNGERIYLSGTGSGISIAQAAMVWDAA

SEQ ID NO: 13 - protein, 346 aa, Desulfobulbus propionicus DSM 2032
MTLRYTQVCLHDFGYQLPPVELSSAAIEERLQPLYERLKLPAGRLELMTGINTRRLWQPGTRPSAGAAAAGADAMAKAGVDVADLGCLLFTSVSRDMMEPATAAFVHRSLGLPSSCLLFDISNACLGFLDGMIMLANMLELGQVKAGLVVAGETAEGLVESTLAHLLAETGLTRKSIKPLFASLTIGSGAVALVMTRRDYRDTGHYLHGGACWAQTVHNDLCQGGQNAEQGTLMSTDSEQLLEKGIETAAACWQQFHATLGWDKGSIDRFFCHQVGKAHAQLLFETLELDPAKNFETLPLLGNVGSVSAPITMALGIEQGALGAGQRAAILGIGSGINSLMLGIDW

SEQ ID NO: 14 - protein, 393 aa, Carboxydothermus hydrogenoformansZ-2901
MREVVIVSAARTPFGKFGGGLSALKAVDLGAIAIKAAVERSGVSPEEFDYVYMGQVLQGGAGQIPSRQAARKAGLPWEVPSVTVNKVCASGLIAVAMAAKMIALGEIDVAVAGGMESMSNAPYILPSARWGQRMFNFEAIDLMVHDGLWCAFYDRHMAVHGSEVAKEYGISRQAQDEWAYISQMRAKEAMEKGRLNDEIVKVEVPGKKGEVVVIEKDEQPRPNTTIEALSKLPPVFDANGTVTAGNAPGVNDGAGALVLMSREKARELGIKPLATYLNHAEVALDAKYIATAPGQAINKLLAKKGMKIEQIDLLEVNEAFAAVVLVSQKIAGYNLEKVNVNGGAVAFGHPIGASGARILMTLIYELRRRGGGTGIAAICSGAAQGDAMLIKVE

SEQ ID NO: 15 - protein, 393 aa, Carboxydothermus hydrogenoformansZ-2901
MQEVVILSAVRTAIGKFGGSLKDIPAAELGAIVIKEALVRAQIPPAEVDEVIFGNVLQAGQGQNPARQAAIKAGIPVDIPAMTVNMVCGSGLRSVSLAATLIAAGEADLIVAGGMENMSAAPYAIPGARWGTRMGDGKIVDLMIKDGLWDAFYDYHMGITAENLAERYNISREEQDRFALESQRRAEKAIKEGRFRDEIVPVKLPQRKGEPLEFVQDENPRFDTTLEALAKLKPAFKEGGTVTAGNASSINDGAAALVIASSKKAESLGIKPMAVIRSWGATGVDPSIMGIGPVGATRKALKRAGLTIADIDLVEANEAFAAQALAVAKELELDLSKTNVNGGAIALGHPIGASGARILVTLLHEMKKSNSRYGLATLCIGGGMGVAAIVEKA

SEQ ID NO: 16 - protein, 435 aa, Syntrophothermus lipocalidus DSM 12680
MSFKKSKDDLVCVSAVRTPFGRFGGSMRDIDIYDLGAIAMKNALERIKMDPELIDEVWWGCGDTTNCKDPYTPVVARQSMLKAGIPPEKPSVTFDQACISGMDAVKYGGRSIQLGEAEIVMTGGATSFSTVPFLLRGIRWEGKRHTSFLVEDPIIPLGYKDYAPVAVDSGDVAVEYGVSREEQDEFAVASHVKYGKAYERGFFKQEMVPLELVKKDKKGNVVSKKVLEIDEQYRPDVKIEELARLKPIFGNPTVTAGNAPGMNDGACAQIFMKREKAEQLGLDVLYTVVAMSSIALQPRIMPVSPAFAIKKCLDVTGLTIDDMKFIEINEAFACVPLVATKLLSNQRFLTSDYNEMVKEASTKPILDNDDSKYQELKSKLNVNGSAIAVGHANTASGSRIMMTAAYNLKENGGGYAACAICGGLTQGAGCIIWVE

SEQ ID NO: 17 - protein, 417 aa, Syntrophothermus lipocalidus DSM 12680
MKDVVIVSACRTAIGTFGGSLKDLNAPTLAKVAMRGAIERAGIDPGLINDVRFGCAFEHPDSNNVARVAALLAGVPAETSTAVTINRVCVSGMEAVVSGMAMIQAGLVDVVLAGGVEHMSGVPFSVLNARWGCRLQDSVFVDNLIHGLYGGSKFLPGPENGPVKEGPILEAGRGKPYIMGYTAELLAQYCNISREAMDEVALRSHNNAERATKDGSFREEIVPVEIPQKKGKAPLVFDKDEHFRPGVTMEQLAALPPAFVPKIGKVTAGNASGMNDGAAAMVIMSADKARELGMKPIARIKAVGYGGCHPSIMGLSPVPAIKNLLSKSGLKLEDFELIEINEAFAAQYLAVEQELGLNREITNVNGSGIGLGHPVGATGCRIMVTLLYAMKKRGKTLGLASLCGGGGVSMAVALEMV

SEQ ID NO: 18 - protein, 393 aa, Carboxydothermus hydrogenoformans Z-2901
MEEVVIVSAVRTPIGSFLGSLAQTPAVDLGALVIKESLNRINLAPRFVDEVIMGNVLQAGLGQNPARQAAIKAGIPQEVPAFTVNKVCGSGLKSVGLAYQAIATGDADIVVAGGMENMSLAPYVLPKARTGYRMGHDTLIDSMIKDGLWCAFTDVHMGITAENIAEKYNITREEQDKFALQSQERAIKAIDEGKFKEEIVPVIIPQKKGEPLVFSTDEFPKRGTSLEKLAALKPAFKKDGTVTAGNASGINDGAAAVVVMSAKKAQELNIKPLAVIRGYAAAGVDPAYMGLGPIPATRKALKKANLTVSDLGLIEANEAFAAQALAVIKELELNPEITNVNGGAIALGHPIGASGARILVTLLHEMQKRNTKYGLATLCIGGGQGFALVVEKV

SEQ ID NO: 19 - protein, 219 aa, Pseudothermotoga lettingae TMO
MKNKAITVEQAIEMIPDGAVLMIGGFLGDGTPELLIDALVKSGKRNFTIIANDTAFPDKGIGKMIVNKMAKKVIVSHIGTNPETQKQMIEGTLEVELVPQGTLAEKVRAGGFGLGGILTPTGVGTVVENGKQKIVIDDKEYLVEPALRADFALIKAQKADFYGNLFFNLTSRNFNPLMAFAGKITIVEVEEFVPVGGLSPNEIHTPHAVVDYIVRGNAR

SEQ ID NO: 20 - protein, 224 aa, Pseudothermotoga lettingae TMO
MIQDQNLAKAVIAKRVALELKDGDIVNLGIGIPTLVANYLPPKVEIFLQSENGILGMGPAPMSGYEHPNLTNAGGSPITFLPGACAFDSAVSFGLIRGGHVDATVLGALQVDEEGHLANWMIPGKMVPGMGGAMDLVTGAKKVIVAMQHVAKGNAPKIVKKCTLPLTSIRRVDLIVTDMAVIEVTGNGLILKELAPQTTVDEVVKFTEAKLIVPEDVPVMPVSL

SEQ ID NO: 21 - protein, 446 aa, Deferribacter desulfuricans SSM1
MAEILKSSIEAIKDVIKDGMVVAAGGFGLCGIPENLINAIKELKVKDLTFVSNNAGVDDFGLGILLQTRQIKKMISSYVGENKIFEQQYLNGELELELVPQGTLAEKLRAGGAGIPAFYTMTGYGTILTEGKEIKVFDGKEYVLEESIRPDLAIVKGWKADKKGNVIFRYTANNFNEVCAKAAKFTIVEVEEIVDEIDPHYIHLPSIYVDRIVLGERYEKRIEQLTTLENMTEAKMNEKREWMAKRVAKELKKGMYVNLGIGMPTLVANFITDDMDITLHSENGLLGIGPFPKTEKDADPDLINAGKQTITYKKGAAFFDSSESFAMVRGGHIDLSVLGGMQVSEKGDLANWMIPGKMVKGPGGAMDLVSGVKKVIVMMEHVAKDGKPKILKECTLPITGKGVVDMLVTDKGVFEINSEGLYLLEISPFSDLEDIKKSTGCEVKVK

SEQ ID NO: 22 - protein, 228 aa, Geobacillus sp. GHH01
MKQIHSSFIEAVKDIPDGATIMVGGFGLVGIPENLILALVETGVKELTVISNNCGVDDWGLGLLLKNKQIKKMIASYVGENKEFERQVLNQEIEVELIPQGTLAERIRAGGAGIPAFYTPAGVGTPIAEGKEVRVFNGKEYILETALVADFSLVRAWKGDKMGNLIYNKTARNFNPMMAAAGKVTIAEVEELVEIGELDPDHIHTPSIYVQRLVVGKQEKRIERLVVR

SEQ ID NO: 23 - protein, 222 aa, Geobacillus sp. GHH01
MKTMNKQSIRERIAKRAEQEIENGFYVNLGIGIPTLVANFIQSHKKVVLQSENGLLGIGPYPLKDEVDPDLINAGKETITAIPGACYFSSAESFAMIRGGHIDVAILGGMEVSEEGDLANWMIPGKMIKGMGGAMDLVHGAKKIIVVMEHVSKDGKPKIVKKCSLPLTGRKVVNRIITEKAVIDVTENGLKLVEILDGSSVEEIQSLTEPTLMIDETLLIQA

SEQ ID NO: 24 - protein, 217 aa, Thermosipho melanesiensis BI429
MKVVDISKINELVKEGATLMIGGFLGVGTPENIIDEIIRHNISNLTVIANDTAFEDRGIGKLVKNKLCKKVIVSHIGTNPETQRQMIEGTLEVELVPQGTLAERIRAAGVGLGGILTPTGVGTVVEKDKKVIEVEGKKYLLELPIHADVALIKAKKADYLGNLVYNLTAENFNPIMALAAKTVIAEVEEIVPTGTLSPNEIKTPGIIVDYIVTGVTR

SEQ ID NO: 25 - protein, 214 aa, Thermosipho melanesiensis BI429
MNPKEKIAIRVAQELKKGQLVNLGIGLPTLVANYIPKDIHVFFQSENGIIGMGPAPKEGYENSDLTNAGASYITALPGAMTFDSAFSFGIIRGGHLDVTVLGGLQVDEEGHLANWMIPGKMIPGMGGAMDLVTGAKKVIVAMTHTAKGTPKIVKKCTLPLTSIRKVDLIVTELAVIEPTDEGLLLKEISKETTLDEVLKLTEAKLIIADDLKIF

SEQ ID NO: 26 - protein, 517 aa, Pelotomaculum thermopropionicum SI
MAPRFLTAEEAVNLIKDGDTVASVGFLGNVFPEELAVALEERFLKTAKPERLTLIYAAAQGDGKERGLNHLAYEGLVKRVIGGHWNLQPKMAKLAIENKIEAYNLPQGTISQLFREIAAKRPGVITHVGLKTFVDPRIEGGKLNAVTKEDIVEVITIDGKEKLFYRSIPLNVGLIRGTSADQLGNISLEKEANTLEVLSIAQAVRNCGGIVIAQVERVVAAGSLDPRLVKVPGILVDVVVVSRPENHHQTFAEVYNPAYSGEVVIPLTELPPAKLDERKVISRRAAFELRPGSVVNLGIGIPEGIASVAAEEGISDFMTLTVEAGPVGGVPAGGLSFGASTNPYCVLDQAYQFDFYDGGGVDIAFLGLAQMDSNGNINVSKFGPRIAGCGGFINITQNAKKVVFCGTFKAGGLKVNVGDGKLTIVNEGKSVKLVPKVEQITFSGEYARQQGQKVLYITERAVFEMTAEGVMLTEIAPGVDLERDVLQQMDFKPLISPSLKTMDKRIFIDAPMGIKNS

SEQ ID NO: 27 - protein, 288 aa, Rhodothermus marinus DSM 4252
MSEPVDHLLHLLNLERIEENIFRGPSRDIGSPTVFGGQVLGQALRAAAYTVPPERRAHSLHAYFILPGDPNAPIVYLVERLRDGRSFTTRRVTAIQHGRPIFNLSASFQIEEPGVEHQDPMPEVPPPEELISEAELRRQLAEQVPEVLRPFLLHERPIEIRPVEPVNLLFPEKRPPRRHAWIRAAGTLPDDDLALHQSVLAYASDFGFMGTAMLPHGLSFLQPHVQAASLDHAMWFYRPFRADEWLLFAMESPVAAHARGLNRGLFFRRDGTLVAAVVQEGLMRIRSD

SEQ ID NO: 28 - protein, 244 aa, Clostridium acetobutylicum ATCC 824
MLKDEVIKQISTPLTSPAFPRGPYKFHNREYFNIVYRTDMDALRKVVPEPLEIDEPLVRFEIMAMHDTSGLGCYTESGQAIPVSFNGVKGDYLHMMYLDNEPAIAVGRELSAYPKKLGYPKLFVDSDTLVGTLDYGKLRVATATMGYKHKALDANEAKDQICRPNYMLKIIPNYDGSPRICELINAKITDVTVHEAWTGPTRLQLFDHAMAPLNDLPVKEIVSSSHILADIILPRAEVIYDYLK

SEQ ID NO: 29 - protein, 353 aa, Thermoanaerobacter brockii Ako-1
MMKGFAMLSIGKVGWIEKEKPAPGPFDAIVRPLAVAPCTSDIHTVFEGAIGERHNMILGHEAVGEVVEVGSEVKDFKPGDRVVVPAITPDWRTSEVQRGYHQHSGGMLAGWKFSNVKDGVFGEFFHVNDADMNLAHLPKEIPLEAAVMIPDMMTTGFHGAELADIELGATVAVLGIGPVGLMAVAGAKLRGAGRIIAVGSRPVCVDAAKYYGATDIVNYKDGPIESQIMNLTEGKGVDAAIIAGGNADIMATAVKIVKPGGTIANVNYFGEGEVLPVPRLEWGCGMAHKTIKGGLCPGGRLRMERLIDLVFYKRVDPSKLVTHVFRGFDNIEKAFMLMKDKPKDLIKPVVILA

SEQ ID NO: 30 - DNA, 1176 bp, Geobacillus sp. GHH01
atgagagaagtggtcattacagcggccgtgcgtacgccgattggaacatttggcggcgtctttaaagacctgttgccgacggatttaattgttcctgttttagaggaagcagtaaaacgcagccaaattgagaaagacgaagtaaacgaagtgattttaggtcattgcattcagagaacggatatacccaatacagcaagaacggctgccttgctagcaggattccctcatacaacaaccggttttacgattcagcgccagtgcgcttctggaatgcaagcagttatttcggcggctatgcaaattcaagtcgggctgagcgatgtggtcattgccggcggtgttgaatccatgagttctagtccgtatatattaaagcagcatcgttggggagcgcgtttacagcaccagcaagtccgtgatagcgtttgggaagttcttgaggatccgattcaccatgtgatgatgggagaaacagccgagaatcttgcggaacggtatgggatcacaagggaggagcaggatgaactggcgttgttaagccatcggcgagctattttggcgatggaatcgggatactttgattctcaaattgttccgatcacggtaaaaacacgaaaggaggagatagtcgtaacaaaagatgagcatccacgagccgatgtgacgaaagaaaaattggcttccttaagacctgtattccgaaaaaatgggacggtaacagcagggaatgcatcgggaattaatgacggtgctgctgcgcttgtgctcatgtctgccgagtatgcacagcaacgagggatcgagccgcttgcaaaagtagttggttattctgttgccggagtggatcctctagtgatgggacgcggcccggttccagcggtacaaaaaggattagaaagggtaaattggacgttagcggaagccgatttaattgaaatcaatgaagcatttgctgctcagtatttagctgtagaaagggaactgcgtttagatagagataaagtgaacgtaaacggaagcggcatcagcttgggacatccgattggatgcacaggagcgcgtattgtcgtcagtcttattcatgagttgcagcgccgtcagcttgaaaaaggaattgcctctttatgcgtgggcggtggaatgggaacggcggtgtttattgaggctttgtaa

SEQ ID NO: 31 - DNA, 1203 bp, Syntrophothermus lipocalidus DSM 12680
atgattaacgaagtcgtaatggtcagtgcatgccgcacggccataggagattttatgggaagcctgaaagatttgaaagccaatgacctgtcagcaataaccgcgaccgaagcactgaaaagagccggaatccagccggaaatggttgatagtcttgttttaggcatgtgcctccaccacggtaacgattccgggccagcgcgccaggtagctatggcgattggcatgagacatagcagctgggcctgcatggtcaatcagaattgcgcctccgccatgcgcgcccttgaaatcgcagccaacgagctcatgctgggcaagagcgagataagcctggttgtgggaacggaaagcatgaccaacgtgccgtacattctgcgtaaagccagattcggctatcgcttgtttgacggtgacaaggccgaggacgctatgatctgcgatggcctgtttgacaaaatggtacccggacacatggcgatcacggccgaaaatgttgccgaaaagtacggaataactagggaagaatgcgatgagctagcgctgttgagccatacccgtgcccttaaggccaacgccgagggtatcttcgcccgggagattgtgccggtggagatcaagaccaagaaaggagtcaaagtagttgacaaagacgaacatcctatggatacaagtctagaaaaattagcccagctacctccggtcttcaagaaaggcggcgtagtcacagccggtaatgcctccggtatcaacgacggttctgcggcggcggtcctcatgaccaagaagaaggccgaagaactcggcatcaaacccttaatgaagcttctatatgtatgcagtgaaggagttgaccccaagtttatgggcttaggaccggcagtagctattcctaaggttctgaataaagcggggttgaagttcgaggatgtggaatactgggaaatcaacgaagcttttgccgctcagtggctgggagtcggccggatgcttaaggaggacttcggaatcgagctcgacctcgacaaggtcaaccataacggctccggcatcggtctcggccatcccgtgggctgtaccggccttcgtattcaagtatccatgtactacgaaatggaaaggctcggtttgaccatcggcggagcttcactctgcgtgggtggtggaccggcaatggctgccctctggacccgggacatataa

SEQ ID NO: 32 - DNA, 1191 bp, Chloroflexus aurantiacus J-10-fl
  misc_feature (1)..(1191): Codon-optimized
atgtccgaaaaacgcgaagtggtggtcctctcaggagtccgtacggccatcggcacgtttggcgggagccttaaggatattcctccgacggaattggccgcgttggtgacacgcgaagccgtggcgcggtcgggtctgcaaccgaatgaaattggccatgtagtctttggccacgtaattaacacggagccgcatgatatgtacctggctcgctacgctgcagtccggggcggacttagcgtggagacgccagccctgacgctcaaccgtttatgtggatcgggcctccaagcgatcgtctcggcggcccagtatatccttcaaggagatgctgaagcggccgtcgcgggtggagcggagtgcatgtcgcgcggaccgtatagcttaccggccatgcgcttcggagcgcgcatgaatgacagcaaagtcgttgacatgatggtcggtgccttgacggatccattcgatgattgtcacatgggcgtcacggccgagaatgtggccgcgaaatggggcatttcacgtgaggatcaggaccaattagcctatgaaagccatatgcgcgcggctcgcgcgatcgacgaagggcgctttgcgaatcaaatcgtgcctgtcgaaatcaaagtcaaagggggcacggcccaattcatggtcgatgagggagtgcgccgggataccacgatcgataaacttgccaaattacggccagtatttctcaaggatgggtcggtcaccgccggaaacgcctcgtcgattaacgatgcggcagccgcggtcgtactgatggatcgtgccaccgctgaacgccgcggttataaacctctggcccgcctggtcgggtattcccatgctgcggtggaaccgaagtatatgggcattggcccggttccggctgttcggcggttgttggaacggacggggttacgcatctcagatatcgacttatttgaggtgaatgaagcctttgctgcccaggctctcgcggtgattcgtgatctcgaactgccaccggatcgcacgaatccaaacgggtcgggcatttctttgggccacccaattggcgccacgggatgcatcctcacggttaaggcaatccacgagttacaccggaccggcggtcgttatgctctggtcacgatgtgcatcggaggtggccagggtatcgctgcgatttttgaacgcatgtaatga

SEQ ID NO: 33 - DNA, 1179 bp, Syntrophothermus lipocalidus DSM 12680
atgcgcgatgtcgtaatagtaagtgggaagaggaccgcaatcggcaattttttaggagcgcttaaagatttttctgcagttgatctaggaacaattgcgctcaaagctgccattaatagtgctggcattagcccggataccgtagaagaagtcgccgccgggcatgtataccaggccggttgtaagggaaatccagcaaggcaaatcaccatcggcgctggctgcccggtggaaacggtttcggttactgtcaatcagcaatgcccttcggccatgcgggccctcgaaattattagccaggaaatcatgctaggcaagatcgacgcgggtgctgccgtaggcatagaaagcatgagtaatgtcccttatctcttgctcaaagctcgcactggttaccgcatgggaaatggagagcttgttgacggcatgctctacgatgcgttgatagacgcgttcggaaacggtcatcaaggaattaccgctgagaacctggcagaaatgtacaatatcagccgagaagaacaggacgagtgggccttcataagccaccagcgtgcctgccaggctatcaaggagggcaagtttaaagacgaaatagtgcccgtcgaagtaaagactaagaaagagactttcctgtttgacaccgacgaacatcctaatccggacaccacgctagaaagcttggctaaactcaagcctgcttttaagaaggacggaaccgtcactgccggaaacgcgtcgtcaattaatgatgcggcgtgtgcagccgtggttatggctcacgacaaggcagtggaattaggaatcaaaccgctcgcccgcatagtggccacggcttcggcggctgttgaaccgcgcattatgggcatcggcgtggtcccggcggtcaaacgggccctcaaatttgcaggaatgagcttagatgatgttcagctttgggagatcaatgaggcgtttgctgcccagttccttgcgtgcaaccgtgaattgaagctcgatacggaaaagattaacgtaaacgggtctgggatctccctggggcatcccgtagggtgtaccggacttcgtttagtaattacccttataaacgagatgaaacgccggaacctgagatacgggtgtgcagccctctgcgcgggtggaggccctgccatggcaaccattatcgaagttctctag

SEQ ID NO: 34 - DNA, 1182 bp, Thermobifida fusca YX
atgagctcccccgaacggatcattgtggttgacggtgcgcggacgccagtcggcagttttggcggcgcgttcaaggatgtgcccgcccacgaactcggtgcggtggcggcccgggcagcgctccagcggtccgggatcgcggcgtccgacatcgacgaggtggtcatgggctgcattggccaggttggcccggacgcttacaacgcgcggcgggtcgctatcgccgctgggcttccggagagtgtccccgcctataccgtcaaccggttgtgcggtagcggtctgcaggcggtgtggtctggggcgatgcagatccgctggggtgcggccgacattgtcctggccggcggtgacgagaacatgagccggatgccgttctacgatttcggggcgcgttccggttatcggctgggggaccgcacgctggtggacggcacggtggcgatgctgacggacccgttctccaacgtgcacatggggtgcacggctgaggcggtggcccgaaagtacggggtgagccgtgctgagcaggatgagtttgcgttggagtcgcagcgtcgcgcggctgctgatgcggcgcgtgccgcgttcgctgaggagatcaccccggtggaggtggggggccgtaagccggtggtggttgaggtggatgagcatcctcggccggacaccacgttggaggggttggcgcggctccgtccggtttttgagaaggacggtacggtgacggcggggaacgcgtcggggatcaatgatggtgcggccgcgttggtcctggctcgtgagtcggtggtgcgtgagcggggcctgaagggtctggctgtggtggagtcggtggcgaccgcggcgatggatccgcagctgatggggtatgcgccggtgcttgcgttgcgcaagctgtttgagcagacggggacgagcccggctgtggttgatgtggtggagttgaatgaggcgtttgcggcgcaggcggttgcggtgatccgggacgctggtctggatccggagaagaccaacccctatggtggggcgattgcgttgggtcatccggtgggggcgaccggggcgattcttacgttgcgggtggcccgggatttggtacggcgtgatcttgagcttggtgtcgtcacgatgtgcattggtggcggacaggctttggccgctttgttgcgtcgggtgtga

SEQ ID NO: 35 - DNA, 1224 bp, Thermobifida fusca YX
atgcctgaagccgtcatcgtcgctacggcacgctctcccatcggacgggctttcaaggggtccctcaaggacatccgcccggacgacctgaccgcgcagatcatctccgcggcgctcgccaaggtcccgcaactggaccccgccaccatcgacgacctcctcctgggctgcgggctccccggtggcgaacagggcttcaacatggcccgcgtcgtcgcggtgcagctcggtctggactcggtcccgggcaccaccatcacccgctactgctcttcatccctgcagaccacccggatggcgttccacgccatcaaggccggggaaggcgacgtcttcatctccgccggcgtggaaatggtcagccgcttcaccaagggcaacagtgacacgctgcccgacacgaagaacccgctgttcgccgaggctgaggcacgcaccgcccgccgcgccgagggcggtgcagaaccctggcgcgacccgcgggaagagggcaagctccccgacatctacatcgctatggggcagaccgcggagaacgtggcgcagctgcgcggcgtctcccgccagcgccaggacgaattcgcggtgcgttcgcagaacctggcggaaaaggcgctcgacaacggtttctgggagcgggagatcaccccggtgaccctgcctgacggcacggtggtctccaccgacgacggtccgcggcgcggcaccacctacgagaaagtcgccgccctggacccggtgttccgccccgacggcacggtgaccgcggggaactgctgcccgctcaacgacggcgcggccgcactgatcatcatgagcgaccggaaggccgctgaactgggcatcaccccgctggcccggatcgtgtcgaccggggtgagcgcgctgtcacccgagatcatggggctgggaccggtcgaggcctcccggcaggcgctggcccgcgccaacatgtcgatccgcgacatcgacctcgtggagatcaacgaggcgttcgccgcgcaggtgcttccgtccgcggacgacctgggcatcgacatcgactcccagctcaacgtcaacggcggcgccatcgctatcggccacccgttcggtatgacgggggcgcggatcaccaccacgctgatcaacgccctccagttccacgacaagaccttcggcttggagaccatgtgcgtgggcggcgggcagggaatggccgccatcttcgaacggctgagctga

SEQ ID NO: 36 - DNA, 1185 bp, Syntrophothermus lipocalidus DSM 12680
atgagcagagaggtcgttttggtaggggcatgtcgcactccgatagggacttttggagggactcttaaggacatgacggcggtacagttgggtacaatcgtcatgaaggaagcattgaagagagccgggatctcaggggaccaggtagatgaggtaatattcggatgtgtgttgcaggcaggacagggacagaacgttgcccgccagtgtgctattcatgcggggataccggaaacggtcacgtcgttcaccattaacaaggtatgcggttctggtctaagagcagtcagccttgcggcgcagatcatcaaagcaggggacgctgacattgtattggctggcggcaccgagagcatgaccaacgctccttatctggttcctaaagcccgttacggatatcgcatgggcgacggcaagctggtggacgagatggtgttcggcgggttgaccgacatcttcaacgggtatcacatggggattaccgcggagaatgtaaacgaaatgtatgggataaccagggaggaacaggacgaatttggattaaggagccaggagagggctttcgcagctatagaatctggcagatttaaggacgagatcgtgccggtagtcatcaagaccaagaagggcgaggtagttttcgatacagatgaacatccccgccgtactacgatggaggccttggccaagctaaaaccggcgtttaagaaagacggcagtgtaactgcaggtaacgcttcggggataaacgacggggcagcggcggtagtagtcatgtcgaaagaaaaggcggacgaactgggaatcaagccgatggccagagtagtaagctatgcctcgggcggggtggatcccaagattatgggtgtaggacctgtacctgctactaagaaagcattagccaaagccggattaaccttagacgatatagatctgattgaagccaacgaagcatttgctgcccaatccatagcggtggcacgcgatatgggctgggacaagatgatggacaaggttaacgtaaacggaggggcaatagccctgggtcatccgatcggagcttctggttgtcgtatactcgtaactctgctctatgaaatgcaaaagaggaacgcgaagcggggactggcaaccttgtgtatcggtggtggacaagggaccacgcttattgtcgagagtttatag

SEQ ID NO: 37 - DNA, 1188 bp, Thermobifida fusca YX
atgcctggatcggtcattgtcggcggcgcacgcacccccatcggaaagctcctcggagcactctccggtttcgccgccgtggacctgggggcgatagcgatcaaagctgccctgcagcgggcagggatctccggtgaccaggtggactacgtcatcatgggccaggtcctgcaggccggccaggggcagatcccctcacggcaggcctccgtcaaagccggaatccccatgagcgtgccttcgctcaccatcaacaaagtctgcctctccggcctcgacgcgatcgccttggctgatcagctcattaccgccggggaattcgacgtggtcgtggccggcggaatggaatccatgacgaatgccccgcacgtcctccccaaagcccgccacggctacaagtacggctccatcgaggtcctcgacgccaccgcccacgacgccctgaccgacgctttcgaccacgtgtccatgggcctgtccacggagcggtacaacgcccgccacggcatgacccgggaagagcaggacgcgttcgcggcgcgctcccaccagcgggccgccgccgctatcgaagcgggactgttcaaagacgagatcgtccccgtcgaagtaccgcggcggaaaggcgaccccacgatcgtcgacaccgacgaaggggtgcgccccgacaccactgtggaagccctggcccggctgcgccccgcgttcgacccggacggcacgatcaccgctggatcctcctcccagatctccgatggcgcgtgcgcggtcgtggtgatgagccggacgaaagcagaagagctgggatgcgagatcctcgcggagatccaggcgcacggcaacgtcgcaggcccggacaactccctgcactgccagccggcgaacgcgatcaagcacgcgctggccaaagcgggacgggatgtcgctgacctcgacctcgtcgagatcaacgaggcgttcgccagcgtggccatccagtccatgcgcgagctgggcgtcagcgaggacatcgtcaacgtcaacggcggagcgatcgcgctaggccacccggtcggcatgtcgggggcacggatcgtgctgcacctcgtccacgagctgcgccgccgcggcggtggactgggtgccgcaggcctgtgcggcggcggcgggcagggcgacgccctcctgctgtcggtgcccgcctcctga

SEQ ID NO: 38 - DNA, 1182 bp, Syntrophothermus lipocalidus DSM 12680
atggctgcgggaatcaaggataaggctgcagttatagggatgggatgcaccaagtttggcgaaagattcgactgtaacctggaggacttgatgttggaggcaatagaagaagccctggccgattcgggacttgagttcaacgatatcgacgctttctggtttggaacttttacctcgggaatggccgggttagccttttccaatcgcatgaaatcacagtacaagccagtgacgcgcatcgaaaacatgtgctgtacggggctggacgcttttcgcaacgcgtgttacgcggtggtgtccggagcctacgacgtagtcatggccattggtgcggaaaaactgaaagacggcggttacagcggtctggaagttcctgccgaggactcagaccgtaccatgcctgacctcactgcgccggcacgttttgcggtgattgcgcccgcctacgctcacaaatatggcctgtccatgcagcagatgaaggaagttatggcccgtattgcctggaagaatcacaagaacggatccttaaacccgaaggcgcaattccaagcagaagttccgattgagaatatacttaagtcccccatgatctgtagtccgttggggataatggactgttctggggtatcggatggggctgcttgcgccatcatagtccgcagtgaggatgctaaaaagtatcgcccggatccgatgtacgtcaaaggtattcagattgcagccgggccggggcacagcgagaagcaccagagttacgacttcaccactgcttgggaaacgtactacgccggacaggcagcgtaccgcgaggccggtataaccaaccctcgggagcagattgacttggctgaggttcacgactgcttcactcccacggagttgattatctatgaggatctccagttcagcgctagaggacaggggtggagagacgcgttggatggcttttttgacttagatggcaagttgccggtcaacccggacggtggcttgaaatcattcggtcatcccataggagctagcgggattcgcatgttgtacgagtcgtggctgcagtttcacggtaaggccggaaagcgccaactggaaaacccgaagataggactggctcataacctgggagggcagccttaccagtgcgtggtgggagtggctgtggtcggcaaggaactgggatag

SEQ ID NO: 39 - DNA, 1182 bp, Chloroflexus aurantiacus J-10-fl
atggacgatgtcgtcattgttggtgcagcgcgtaccccaatcgggcgcttcaacagcgcctacagcggattgagtgccatcgatttaggtgcagccgccgtgcaagcggctgtccaacgggccggaattgaggcagactccatcgatgaatgcattatgggctgcgtagtcaccgccggcctgggacaatcaccggcacgccaggcagctctgcgtgcgggcttgccgcatacaattggcggcctgaccatcaacaaggtctgtggcagtggcctcaaggcagtcatgatcggcaccgccctgatcaaagccggcgaagctgatgtcattgtcgccggcggtatggagcacatgagcggtgcgccatacctgcttccccaggcccgccacggctaccggctcggccacggccagatcatcgacgctgtcgttcacgatggtctgtggtgcgcttttgagcatcatcacatgggagtggccgctgaatggattgcgcgcaccttcaatgtcactcgcgaacagcaagatgcttacgcattgcaatcacaccaacgcgcagtagccgctcaggacagcggcgccttccaggccgaaattgcaccggtaaccgtcccagggccgaaaggccaggtcaatctggtgacgactgacgaaggccccagacgcgacacctcgctggctgcactggcaaagctcaaaccggcatttgtcaccgacggcaccgtcactgccggcaatgcccccggcattaccgacggcgcggcagcagtcgtactaatgcgagccagccgggcagcccaattgggggtgcaacccttagcccgcatcggcacagccgcccaggccgccgtcaagccgcttgaactcttcaccgcaccggcgtttgccatcgaacggctgatgaagcgggcaggccgtaccctcgacgactacgacctgttcgagatcaacgaagcctttgccgcccaggtcattgcgaacctgcgtgccctggccctcgatgcagaccgggtcaatgtccacggtggcgcgattgctctcggccatccgataggagccagcggagcacgtgtcctggtgacactcatctcagcgttacgccagcgcggcggccagcgcgggattgccgcactgtgtctgggaggtggtgaagcggtcgccctcgaagtcgaggtcgtctaa

SEQ ID NO: 40 - DNA, 1143 bp, Thermobifida fusca YX
  misc_feature (1)..(1143): Codon-optimized
atggccgaggcatacatcgtcggagcggtgcgcaccccgatcgggaccaggaagggggcgctcgctgcggtgcacccggccgacctgggcgcccacgtgctcaaagaactggtgaaccggaccgggatcgacccggccgcggtcgaggacgtgatcatgggctgcgtcacccaggtggggccgcaggcactcgacctggcccgcaccgcatggctttctgccggactcccggagagtacgccgggggtcaccatcgaccgccagtgcggttcctcccagcaggccgtgcactttgcggcgcaaggggtcatgtccggcacccaagacctggtgatcgcggcgggtgtggagaacatgggcatggtccccatgggcgccaacgtgcagttcgccgtggacaacgggttgtccgtctacgggcagggctgggtcgaacggtacggcacccaggagatctcccagttccgcggggcccaactgatgtgtgaaaagtgggggtacacccgcgaggacttggagaagtacgcgctggaaagccaccgtcgggctgccgcggcgatcgaagcgggctacttcgacgcggagactgccccgctggccggggtcacccacgacgagggggtgcgccccgacacgtcgctggagaagatggccgagctcgcaccgctccgcgaaggctgggcgttgaccgcggccgtctccagccagatttcggtgggggcgagcgccctgctcatcgcgtcggagcgggccgtggccgagcacgggctcacccccttggcgcggatcgtgcaactggctttggccggggacgacccggtgtacatgctcaccgcgccgatccccgccactcggatcgcgctgcgcaaggccgggctggacatcgacgacatcgacgtggtcgagatcaacgaggcgttcgccccggtccccatggcgtggatcgacgaaatcggcgccgacccggcgaaggtcaaccccaacggcggtgcgatcgccctgggccacccgctgggggccaccggcgccgtgctcatgaccaagctcgtccacgaactgcgccgcacgggcggccgctacgggctgcagaccatgtgcgagggcgggggacaggccaacgtcaccatcatcgaacgggtgtga

SEQ ID NO: 41 - DNA, 1029 bp, Sulfurifustis variabilis
atgcacagcgttggccactcgcggatcatcagcacggggatgtatctcccgccggagcggctctcttcgagagagctcatggagatgttccgatcgcgggagcgatttggactcccctacgagtggctcgagcgcaccaccggcatccgcgagcggcgcttcgcgccgcccgatttcaaatcctcggagatggccgtcgcggcggcccgcgaggcgctggaactcggcgaggtctcgccgtcccagatcgacgcgatcatttactgcggggtgctccgcgaccacgtcgagcccgccacggcacacgtggtccaggacaagatcggcgcgcgcaatgccatcgccttcgacgtctcgaacgcctgcctcgggttcatgaacggcatgcatttgatggacgcgctgatcgccaccggccaggccaggcggggtctcgtcgtaacgggcgagcgaggcaaccactacatccgcaaggcgctccgggtgcttgcggaactgccggacaacggcgatttcagcgacctggccgccgccctcaccctgggtgacgcaggggccgcggccgtcatgggtcccaagctcgacccggagaccggcatcaagggcttcgttgtacagtcgcagggacagcacaacgggctgtgcgtgtgcggggacaacggtgaggacacgcatctggtcaccaaaatcacggagatcgtgagggagaccacgaggctggtgggcccgttgtaccaggccctcatgcatgagcacctcgggtggcaaccctcggagctgagtcgctatatcccccatcaggtcggattgcgctccgtgcgcaagcacgccgaggtggcccaagtcccgctggaaatcatcccgattacggtcgattacctcggaaacatcatttcggccaccatacctgtaaatatctcgttgttaatgaaggataaaaagctaaccaacggggaaaggatctatctttccgggacgggcagcgggatcagcatcgcccaggccgccatggtatgggacgccgcctga

SEQ ID NO: 42 - DNA, 1041 bp, Desulfobulbus propionicusDSM 2032
atgactttgcgttacacccaggtctgtttgcacgacttcggctatcaactgccgccggtggagttgtcttcggcggcgatcgaggagcggcttcagcccctctatgagcggctgaagctgccggccggtcgactggagctgatgaccgggatcaacacccggcgtctgtggcaacccggcacccggccaagcgcaggggcggcagccgctggagcagatgccatggccaaggccggggtggacgtggccgatctcggctgtctgctctttacctcggtgagccgcgacatgatggagccggccaccgccgcctttgtccatcgcagcctggggctgccctcgtcctgtttgctgtttgacatttccaacgcctgtctcggctttcttgacggcatgatcatgctggccaacatgctggaattgggacaggtcaaggccgggttggtggtggcgggcgagaccgccgagggtctggtcgaatccaccctggcccatctgctcgccgaaaccggactgacccgcaaatcgatcaagcctctctttgcctccctgaccatcggctcgggggccgtggccctggtgatgacccggcgtgactaccgggataccggccattatctgcacggcggcgcctgctgggcccagaccgtccacaacgatctttgccagggcgggcagaatgccgaacagggcacgctcatgtccaccgattccgagcagctgctggaaaagggcatcgagaccgcggccgcctgctggcagcagtttcacgccaccttgggctgggacaagggttccatcgaccgcttcttttgtcatcaggtcggcaaggcccacgcccaactgctgttcgagaccctggaactcgatccggccaagaatttcgagaccctgcccctgttgggcaacgttggttcggtgtccgcgcccattaccatggccttgggcatcgagcagggcgccttgggtgccggacagcgggccgccatcctgggcatcggctcgggcatcaattcgctcatgctgggcatcgactggtaa

SEQ ID NO: 43 - DNA, 1182 bp, Carboxydothermus hydrogenoformans Z-2901
atgagagaagtagttattgtaagtgcggcccgaacaccctttgggaagtttggtggaggactttcggctttaaaagcggttgacctgggggcaatagctatcaaggcagcggtagagagaagcggagtaagtccggaagagtttgactatgtttacatgggtcaggttttacagggaggagcgggtcaaataccttcccggcaggcggcaagaaaagcgggtctaccctgggaagttccgtcagtaacggtaaataaagtatgtgccagcggtttaatcgcggtagctatggcggcaaagatgattgctttaggcgaaattgacgtggcagttgcaggcggaatggaaagcatgagtaatgccccatatatattgcccagtgcccgctggggacagagaatgtttaattttgaagctatagatttaatggtgcatgatggtctctggtgtgctttttatgaccggcacatggcggttcacggctcggaagttgccaaggaatatggtatttcccggcaagctcaggatgaatgggcatatattagtcaaatgagggctaaagaagcaatggaaaaaggacggctgaacgatgaaattgtcaaagttgaggtacccgggaaaaaaggagaggttgttgtcattgaaaaagatgaacagccgcgtcccaatacaacgattgaagctctttctaaacttccgccggtttttgatgccaacgggaccgttactgccggaaatgctcccggtgtaaatgatggagcaggagctttggtcctaatgagtagagaaaaagcccgggaacttggaattaaacctctggcgacttaccttaaccatgccgaagtagctttagatgccaaatatattgcaactgcaccgggacaggcgattaacaagcttttagcgaagaaaggaatgaaaattgaacaaatagatcttttagaagttaatgaagcttttgcggcggtagttttggtcagtcaaaaaattgccgggtataaccttgaaaaagttaatgttaacggtggagctgtagcttttggtcatcccatcggtgcaagcggggcccgtattttaatgaccctgatttatgagttaagacgtcggggtgggggaacaggaatagctgccatttgcagcggagcggcccaaggggatgccatgttgattaaagtggaatag

SEQ ID NO: 44 - DNA, 1182 bp, Carboxydothermus hydrogenoformans Z-2901
atgcaggaggtagtaattttaagtgcggtgaggactgctataggcaaattcggaggtagcttaaaagacattcctgctgcagaattgggggctatcgttataaaagaagctttggtgagagctcaaataccacctgcagaggtagatgaagttatttttgggaatgtcttacaagctggacaagggcaaaatccagcgcgtcaggcggctattaaggcaggcattccggtagatattccggcaatgactgtaaatatggtttgtggctccggtttacggtctgtcagtttagcagctactcttattgctgcaggggaagctgatcttattgtcgcaggcgggatggaaaatatgtctgctgctccctatgccatacccggagcacgttggggtacacgcatgggggatggaaagattgttgatttgatgattaaggatggtctatgggatgctttttatgactaccatatggggattactgctgaaaatttggcagaacgctataatataagccgggaagaacaagacagatttgctttagaaagtcaacggcgagctgaaaaagcaattaaagaaggacgtttccgtgatgagattgttccggtgaagttacctcagcggaaaggagaacctcttgaatttgttcaagatgagaacccgcgttttgatactactcttgaagcgttagcaaagttaaagccagcatttaaagaaggtggaacagtaactgccggcaatgcatcaagcataaatgacggtgctgcggcattggtaatagcttccagcaaaaaagctgagagtttgggaattaaaccaatggctgttattcggagttggggagctaccggggtagatccaagtattatggggatcggtcctgttggagctactcgtaaagcgttaaagagagcaggcttaacaattgctgatatagatttagtagaagctaatgaagcttttgcagctcaagcccttgcagtagctaaggagttagaacttgacttaagtaaaactaatgtaaatggtggtgctattgctttaggtcacccgattggtgcaagcggggcaagaattcttgtaactttgcttcatgagatgaaaaaatcaaatagccgctatggcctggctacgttgtgcattggtggcggtatgggagtagcagctatagtagaaaaagcttag

SEQ ID NO: 45 - DNA, 1308 bp, Syntrophothermus lipocalidus DSM 12680
atgagttttaagaagagcaaggacgacctagtatgtgtatcagcggtaaggactccgttcggtcgttttggcgggtcaatgcgagacatcgatatctatgacctaggcgccattgccatgaagaacgccctagagcggataaaaatggaccccgagttgatcgacgaagtctggtgggggtgtggcgataccactaactgtaaagacccctacaccccggttgttgctcggcaaagcatgttgaaagccggtattccaccggaaaagccgtctgtaacgtttgaccaagcctgcatctctgggatggatgcggtgaaatatggaggccggagcatacaactgggtgaagccgagattgtcatgactggtggtgctactagctttagtacggttccattccttctccgcggcatacggtgggaaggaaaacgtcacacgtcatttctagtggaggatcccataattcctctaggatacaaggattacgctccggtggcagttgactctggggacgtagctgttgaatacggagtatccagagaggaacaggatgagttcgcagtggccagtcacgttaagtacggcaaggcctacgaacggggtttcttcaagcaggaaatggttccgctggaattggtcaagaaggataagaaagggaatgtagtgtctaaaaaggttttggagattgacgagcaatatcgccctgacgtcaaaattgaagaactagccaggttaaagcctatatttggcaatcctacggtaacggcgggtaacgccccgggcatgaacgacggggcttgcgcacaaatcttcatgaagcgggaaaaggccgaacagttagggctggacgtcctgtatacagtggtagccatgtcgtcaatagcgcttcaaccccggattatgcccgtatccccggcatttgcgatcaaaaagtgcttggacgtgaccgggttaaccatcgacgatatgaagttcatcgagattaacgaggcctttgcttgcgttccactggtggcaacaaaactcctgtccaaccagaggttcctgactagcgactacaacgagatggttaaggaagcgtcgaccaagcccatcctagataacgacgatagcaagtaccaggaattgaagagcaagctaaatgtcaacggcagtgccattgcggtggggcatgctaatacggccagtggttcacgcatcatgatgactgctgcctataacttgaaggaaaacgggggcggctacgctgcgtgtgcaatatgcggcgggctgactcaaggagcaggttgcataatctgggttgaataa

SEQ ID NO: 46 - DNA, 1254 bp, Syntrophothermus lipocalidus DSM 12680
atgaaagacgtggtcatagtgtccgcctgccggacagctataggaacctttgggggttcgttgaaggacctaaatgctccgaccctggcgaaggtagccatgcggggagcaatcgagagggcagggatagatccggggctaattaatgatgtgcgctttggttgtgcgtttgaacatcctgacagcaataacgtagctcgtgtcgcagcgctgttagcaggagtacctgctgagacatcgactgcggtgacgataaacagggtttgtgtgtcgggaatggaagcggttgtatcgggtatggccatgatccaggcaggccttgtcgatgtggttttggccggtggggtggagcacatgtcaggtgtcccgtttagcgttctaaatgccaggtggggctgtcgccttcaggattcggtttttgtcgataacctgattcacgggttatacggcgggtcaaagtttttgccagggccggaaaacggtccggtcaaggaaggtcccattctggaggcgggtcgaggtaagccttatattatgggttacacggcggagttattagcccaatactgcaatatcagtcgcgaagcgatggatgaagttgccctgcggagccataacaacgcagagcgcgccacaaaggatggatcgtttcgagaggagattgttccagttgaaatcccacagaaaaaggggaaggctcccttagtgtttgacaaggatgagcattttagaccaggcgttactatggaacaacttgctgctttaccgccggcttttgtccccaagatcggaaaagtaacggcaggcaatgcttccgggatgaatgacggagccgcagctatggtaataatgtcggctgataaggctagagaattagggatgaaaccaattgccagaattaaggcggtcggctacggaggatgccatccatctatcatgggattgagcccggttccggctataaagaatctgctgtcaaaatcggggcttaaattagaagattttgaactcatagaaattaatgaagcatttgcggctcagtatctcgccgttgagcaggaattgggcttaaatcgcgagattaccaacgtcaatggatctgggatcggtttggggcacccggttggagctaccggatgccggattatggtgacgctgctgtatgcgatgaaaaagagaggcaagacactggggttagcaagcctgtgcggcggcggcggagtatcgatggcggtcgctttggagatggtttaa

SEQ ID NO: 47 - DNA, 1182 bp, Carboxydothermus hydrogenoformans Z-2901
atggaagaagttgtaatagttagtgccgttagaactcccattggcagttttttgggtagccttgcccaaactccggcagtggatttgggagcgcttgttattaaagaaagtctaaaccgcattaaccttgcgccccggtttgtcgatgaggttatcatgggaaacgttttgcaggcaggattaggccagaacccagcccggcaggcagctataaaagcaggaataccccaggaagtacctgcttttacagtaaacaaagtttgcggttcaggattaaaatccgtcggactggcttatcaggcaatagcaacaggtgatgccgatatcgttgtcgccgggggaatggaaaacatgtctttagcaccttacgtcttgcccaaagccaggacaggttaccgcatggggcatgataccctgatagattccatgattaaagatggcttatggtgtgcttttaccgatgtgcatatgggtattaccgcggaaaatatagccgaaaaatacaatataacccgcgaagaacaggataaatttgccctgcaaagtcaggaaagagctataaaagcaattgatgaaggaaagtttaaagaagaaatcgttcccgtaatcatcccccagaaaaaaggagaacccctggtattttccaccgatgaatttcccaaacgcggtacatccctggaaaaacttgccgctctaaaaccggccttcaaaaaagatggtaccgtaactgccggaaatgcctcgggaattaacgatggagctgctgcggttgtagttatgtcagcaaaaaaagctcaagagttaaatattaaacccttggctgttatccgcggttatgcagctgcgggagtagatcctgcctatatgggtttaggcccaatacctgccacccgcaaagcccttaaaaaagccaatttaaccgtttcggacttggggcttattgaagcaaacgaagcttttgccgcccaggctttagcggtaattaaagagctcgaattaaatccggaaataactaatgtcaacggtggtgccatagcgttaggtcaccccataggagcctcgggagctcgaatattggtaaccttattacatgagatgcaaaaacgtaatacaaaatacggtttagcaaccttgtgtatcggtggcggccaaggatttgctttagtagttgaaaaagtttaa

SEQ ID NO: 48 - DNA, 663 bp, Pseudothermotoga lettingae TMO
  misc_feature (1)..(663): Codon-optimized
atgaaaaacaaggcgattaccgtcgaacaagcgattgagatgatcccggatggcgcggtccttatgattgggggatttcttggtgacggcacgccggagttgctaattgatgcgttggtcaaatcagggaagcggaatttcacgattattgccaacgatacggcctttccggacaagggcattggcaagatgatcgttaacaaaatggcgaaaaaggtcatcgtgtcgcatattgggaccaatcctgaaacacagaaacaaatgatcgagggcacccttgaggttgagttggtgccgcaaggaacattagcggaaaaagttagagccggcggctttggccttggcggcattttgacgccgacaggcgtggggacggtcgtcgaaaatggaaaacagaaaatcgtgatcgacgataaggaatatcttgttgaaccggctttacgagcagactttgcactaattaaagcacaaaaagcggacttctacggaaatctgttctttaatttgacatcccgtaactttaacccgctcatggcgtttgctggcaaaataacaattgtcgaggtggaagagtttgtacctgttgggggactttctcctaatgaaattcacacgccacatgcggtggtggattatattgttcgggggaacgctcggtaatga

SEQ ID NO: 49 - DNA, 678 bp, Pseudothermotoga lettingae TMO
  misc_feature (1)..(678): Codon-optimized
atgatccaagaccaaaacctggccaaagctgtcatcgcgaaacgtgtggcattggaattaaaagacggggatattgtcaatttaggaattgggatccctacgctcgtagcgaattatcttccccctaaagtagaaatcttcctccaatcagaaaatggcatcctgggtatgggtcctgctccaatgtcgggctatgagcatccaaatttgacgaatgccggcgggtcgccgatcacgtttttgccaggtgcctgcgcatttgacagcgccgtctcctttgggttaatccgcggggggcacgtcgatgcgacggttctcggcgccctccaagtggatgaagaagggcacttggccaactggatgattccgggcaaaatggtgccgggcatgggtggcgctatggacttggtgacaggcgcgaagaaagtcatcgtcgccatgcaacacgtcgccaaaggcaatgctccgaaaatcgtgaaaaagtgcacgctgccgctcacgagcattaggcgcgtcgacttgattgttacggatatggcggtgattgaagtcactgggaatggtttaattcttaaagaacttgctccgcaaacaacagtcgatgaggtcgttaaatttacggaagcgaaactcattgtcccagaggatgtgccagtgatgccggtaagcctctaatga

SEQ ID NO: 50 - DNA, 1344 bp, Deferribacter desulfuricans SSM1
  misc_feature (1)..(1344): Codon-optimized
atggccgaaattttgaaatcgtctatcgaggccattaaagacgtgattaaagatggaatggtggtggccgccgggggcttcgggctctgcggtatcccagaaaacttgatcaatgcaataaaggagctcaaagtcaaggatctgacatttgtgagcaataatgcgggtgtagatgattttggccttggaattctcctccaaacgagacaaatcaaaaagatgatttcgtcgtacgtgggcgaaaataagatttttgaacagcaataccttaacggggaactggaattggagttggtcccgcaaggcacccttgccgagaaattacgtgcgggaggtgcagggattcccgcgttctacacgatgacgggctacgggacaatccttactgaagggaaagaaatcaaagtattcgatggcaaagaatatgtgctggaagaaagcattcgccctgatttagccatcgtgaaaggctggaaagccgacaaaaaaggcaatgtaatcttccggtacactgccaataattttaatgaagtgtgtgcgaaagccgcgaagtttacgattgtcgaagttgaagaaatcgttgatgaaattgacccgcactacatccaccttccgtcgatctacgtcgatcgaattgttctcggcgaacgctacgagaaaagaatcgaacaacttacgaccttagaaaatatgacagaagcgaaaatgaacgagaaacgtgaatggatggccaaacgcgtggcgaaagagttgaaaaagggtatgtatgttaacctcggcattggcatgccgacgttagtcgcgaactttatcaccgacgatatggatataacgctgcactcggaaaacgggttacttggcataggtccgtttccaaaaacggagaaagatgctgacccggaccttatcaacgccggtaaacaaacaatcacgtataaaaaaggcgcggctttttttgattcaagcgaatcgtttgctatggtccggggcggacatattgatctctccgtcctgggcggaatgcaggtcagcgaaaagggcgaccttgcaaactggatgattccgggtaaaatggtgaaaggaccggggggagccatggacttagtctccggcgttaaaaaagttatcgttatgatggaacatgtggctaaagatgggaagccgaagattctaaaagaatgcacgttgccgattaccgggaaaggtgttgtggatatgttggtgactgacaagggtgtgttcgagatcaattctgaggggttgtaccttttagaaatctctccatttagtgaccttgaagacattaagaagagcaccgggtgtgaagtgaaagtcaaataataa

SEQ ID NO: 51 - DNA, 687 bp, Geobacillus sp. GHH01
atgaagcagatacattcttcttttattgaggcggtgaaagacattccggacggagcaacgattatggttggcggcttcgggcttgtcggcattccagaaaacttaattctcgcgctagtggaaaccggggtcaaggaattaacagtcatctctaataattgtggcgtggatgactggggacttggattgctcctgaaaaataaacaaattaagaaaatgatagcttcctatgttggagaaaataaggagtttgaacgccaagttctcaatcaggaaatagaagttgaattaattccccaaggaacgttggcagaacgcattcgcgccggcggggccggaataccggcattttatactcctgctggagttggcaccccgattgcggaagggaaagaagtacgagtatttaacggcaaagagtatattcttgaaacggcgttagttgctgactttagtttagtgcgcgcgtggaaaggagataaaatggggaatttgatttacaacaaaacagcgcgtaactttaacccgatgatggcggcagcagggaaagttacgattgcagaagtggaggaacttgtggaaattggagaattggatccggatcacattcatacgccaagcatttatgtacaacgattagtagttggaaaacaagaaaaacggattgaacgtctagttgttcgctag

SEQ ID NO: 52 - DNA, 660 bp, Geobacillus sp. GHH01
atgaataaacaatccattcgtgaaagaattgccaagcgtgctgaacaggagattgaaaacggtttctacgtcaatttagggattggaataccaactcttgtcgccaattttattcaatcgcataaaaaggtggtgctgcagtccgaaaacggattgttagggattggaccttaccctctcaaggatgaggtagaccccgatttaatcaatgccgggaaagaaacgataacggctattcccggagcttgctattttagcagtgccgaatcatttgccatgatccgtggcggtcatatcgatgtagctattttaggaggaatggaagtttcggaagagggtgatcttgctaattggatgatccctggaaaaatgattaaaggcatgggaggagcgatggatctagtgcatggagcgaaaaagattattgttgttatggagcatgttagcaaggatggaaaaccgaagattgtgaaaaagtgtagtcttccgttgacaggaaggaaagtggtcaaccgcattattaccgaaaaagcggttatcgatgtgaccgagaatggcttgaagttagtagaaattttggatggaagtagcgtcgaagagattcaatctctgacagaaccaacattgatgatcgatgaaacgcttcttattcaggcataa

SEQ ID NO: 53 - DNA, 654 bp, Thermosipho melanesiensis BI429
  misc_feature (1)..(654): Codon-optimized
atgaaagtggtagacatctctaagatcaacgagctggtaaaagagggtgcaacattgatgatcggtggttttctcggtgttggcacaccggaaaatatcattgatgagatcatccggcataacatttctaaccttacagtgattgctaacgatacagcttttgaagaccggggtattggtaaattagtaaagaataaactctgcaagaaggtaattgtgtcccatatcggaacaaacccggaaacacaacgccagatgattgagggcacactggaggtggagcttgtaccgcagggaacccttgccgaacgtatccgcgccgctggggtagggcttgggggtatccttacgcctacaggtgtaggcacggtggtggagaaagacaagaaggtgatcgaagtggaaggcaaaaagtacttacttgaacttccgatccatgcggacgtcgcccttatcaaagcgaaaaaggcagactatctcggtaaccttgtctataacctcacggctgaaaattttaaccctattatggcccttgcggcaaagacagttatcgcagaggtcgaggaaatcgtgccaacgggcacattatctcctaatgagatcaaaacgcctgggattatcgttgattacatcgtaacaggggtcacacgttag

SEQ ID NO: 54 - DNA, 645 bp, Thermosipho melanesiensis BI429
  misc_feature (1)..(645): Codon-optimized
atgaaccctaaagaaaaaatcgctattcgcgttgcacaagaactcaaaaagggacagttagtaaacctcggaatcggattaccaacgcttgtagcgaactacatcccgaaagatattcatgtcttcttccagtccgagaatggtatcattggaatgggccctgcgccgaaggagggatacgagaactcggatttaacgaatgccggtgcgagctacattacggcccttccaggtgcgatgaccttcgattctgcgttctcgtttggaattatccggggtgggcaccttgacgttacagttcttggaggtttacaagttgacgaggaggggcaccttgcgaattggatgatccctgggaagatgattcctggaatggggggcgctatggatctggtaacaggggctaagaaggtcattgtagccatgacccacaccgcaaagggtaccccaaaaatcgtcaagaagtgtacattaccacttacatccatccgcaaagtagatcttattgtaacggagttagcagttattgaaccgacagacgagggcctcttgctgaaggagatctctaaggaaacgacactggatgaagttctcaaattgacagaagctaagttaattattgccgatgacctgaaaatcttctaa

SEQ ID NO: 55 - DNA, 1557 bp, Pelotomaculum thermopropionicum SI
  misc_feature (1)..(1557): Codon-optimized
atggcgccacggtttttaactgccgaagaagccgtcaacctgattaaagatggcgacacggtcgcgtccgtggggttcctcggaaatgtgttccctgaggagttagctgtggcattggaggaacgctttctgaaaacggccaaaccggagcgcctgactcttatctatgcagcggcacaaggtgatggcaaggaacgcggccttaatcatctggcctatgaaggtttggtgaagcgggtcattggtggtcattggaacttgcaaccaaagatggccaaattagccatcgagaataaaatcgaagcttataacttaccgcaaggcaccatcagccaacttttcagggagattgcggccaaacgtccaggggtcatcacgcatgtaggacttaaaacgtttgtcgaccctcgcattgaggggggtaaactgaatgccgtgactaaagaagacattgtcgaagtcattacaattgatggaaaagagaaattgttttaccgttctattccgctcaatgtagggttgattcgcggcacatccgcagatcagttgggcaatatttcgctcgaaaaagaggcgaataccctcgaagtccttagcatcgcccaagcggttcgcaactgcggtggcatcgtgattgcccaggtagaacgcgtagtagcagctggctcgttagacccacggttagtaaaagttccgggtattttggtagacgtggtcgtcgtctcacggcctgaaaaccatcatcaaacttttgctgaagtatacaaccctgcttacagtggagaggtagtcattcctctgaccgaacttccgccggctaaattggatgaacgtaaggtgatcagccgtcgcgccgctttcgaacttcggccgggcagcgtggttaacttggggatcgggatccctgaaggtattgcgtctgtcgcggcagaggaagggatcagcgacttcatgacactgacggtcgaagccggaccggttggcggcgttccagcgggcggcttgtcatttggggcttcgacgaatccgtattgcgtgctcgatcaagcctatcaattcgatttctacgacggaggcggagtagatattgcctttttgggtttggctcaaatggatagcaacggaaacattaacgttagtaaatttgggcctcgtattgcaggatgtggcgggtttatcaacattacgcaaaatgcgaaaaaagtggtattctgcggcacgtttaaggccgggggcttgaaagtgaacgtgggagatggaaaattaactattgtgaacgagggaaagtccgtgaaattggtgccgaaagtcgagcagatcacctttagtggtgaatatgctagacaacaaggccagaaagtgctgtatatcaccgaacgcgctgtctttgaaatgacggccgaaggtgtgatgttgactgaaatcgctcctggcgtcgacttggaacgcgatgtcttgcaacagatggactttaagccactgatctcaccgtcgttaaaaacaatggacaaacgcatctttatagacgcgccgatgggaattaaaaattcctgatga

SEQ ID NO: 56 - DNA, 870 bp, Rhodothermus marinus DSM 4252
  misc_feature (1)..(870): Codon-optimized
atgtccgaaccggtcgaccatctacttcatctgcttaatttggaacgcatcgaagagaatatttttcgaggaccgtctcgtgatattggctcgccaacggtgtttggtgggcaagtgcttggccaggcgttacgggccgccgcctacactgtgccgccagagcgtagagcccatagcttgcatgcctattttattcttccaggtgatccgaacgcgccgattgtatatctagtggagcggttacgcgatggccggtcgtttacgactcgcagagtaacggcaatccaacatggccggccgatctttaacctctcggcgagctttcaaattgaagaaccaggagttgaacatcaggatccgatgcccgaggtgcctccgccggaggaacttatttccgaagcagagctacgccggcagcttgctgaacaggtgccggaagttttaaggccattcttgctgcacgaacgtccgattgaaatacggccggtcgagccggtcaatttattgtttccggagaaacgtccgccacgcaggcatgcgtggattcgagcagcagggacgcttccggatgacgacttggccctccatcagtcagttttagcctatgcttcagattttggtttcatgggtacggcgatgttaccgcacggcttgtcatttctgcaaccgcatgttcaagccgcatcattggatcacgccatgtggttttatcgtccgtttcgggcagacgaatggctgttgttcgccatggaatcaccggtcgcggcccacgcacggggcttaaataggggcttgttttttaggcgtgatgggacgctggtagcagcggtcgtccaagaaggacttatgcggattcgctcggattaatga

SEQ ID NO: 57 - DNA, 735 bp, Clostridium acetobutylicum ATCC 824
atgttaaaggatgaagtaattaaacaaattagcacgccattaacttcgcctgcatttcctagaggaccctataaatttcataatcgtgagtattttaacattgtatatcgtacagatatggatgcacttcgtaaagttgtgccagagcctttagaaattgatgagcccttagtcaggtttgaaattatggcaatgcatgatacgagtggacttggttgttatacagaaagcggacaggctattcccgtaagctttaatggagttaagggagattatcttcatatgatgtatttagataatgagcctgcaattgcagtaggaagggaattaagtgcatatcctaaaaagctcgggtatccaaagctttttgtggattcagatactttagtaggaactttagactatggaaaacttagagttgcgacagctacaatggggtacaaacataaagccttagatgctaatgaagcaaaggatcaaatttgtcgccctaattatatgttgaaaataatacccaattatgatggaagccctagaatatgtgagcttataaatgcgaaaatcacagatgttaccgtacatgaagcttggacaggaccaactcgactgcagttatttgatcacgctatggcgccacttaatgatttgccagtaaaagagattgtttctagctctcacattcttgcagatataatattgcctagagctgaagttatatatgattatcttaagtaa

SEQ ID NO: 58 - DNA, 1062 bp, Thermoanaerobacter brockii Ako-1
  misc_feature (1)..(1062): Codon-optimized
atgaaaggatttgcaatgttatccattggaaaggtaggttggatcgaaaaagagaagccagcgcctggaccatttgatgctatcgtccgcccgttggcagttgcaccttgcacctccgacattcacaccgttttcgagggcgcgatcggagagcgccataacatgattttgggccatgaggctgttggtgaagtagtagaggtgggttccgaggttaaagattttaagcctggtgaccgggtagtggtccctgccattaccccagattggcgtacgtctgaagtacagcgtggttaccaccagcactcggggggaatgttggcaggatggaagttttcgaatgtaaaagatggcgtattcggtgaattttttcatgtaaacgacgcggatatgaacttggctcaccttccgaaggaaatcccgcttgaagcggcagttatgatcccggacatgatgaccaccggctttcacggtgcggagctggccgacatcgagttaggcgctacggtagccgtacttggtatcggtcctgtaggactcatggccgttgctggcgcgaaactgcgtggagcgggccgcatcatcgccgtcggatcccggccagtctgtgtggacgcggcaaaatattatggagcaacagacatcgtcaattataaggatgggcctatcgagtcgcagatcatgaacttaaccgagggcaaaggcgtagatgccgccatcattgcaggtgggaatgctgacattatggctacagccgtgaaaatcgttaaaccgggtgggaccatcgcaaatgtgaattactttggggagggggaagtcctgccggttcctcggcttgagtggggatgcggaatggctcataagacgattaagggaggcttatgtcctggtggccgcttgcgtatggagcgtttgattgatctggtcttttacaagcgcgtcgatccgtcgaagcttgttacccatgtctttcggggatttgataacattgagaaggccttcatgctcatgaaagataagccaaaggatcttatcaagccggtcgtcatcttagcgtagtga

SEQ ID NO: 59 - protein, 391 aa, Deferribacter desulfuricans
MRDVFVVEGLRTPFGSFGGSLSDVHPAVLAADVIKKLLEKTEVKPDDIDEVILGQVLTGGFGQAPARQAMRYAGLLDKVHAMTINKVCGSGLKALMLGAQSIMLGDSDLAIVGGMENMSMAPYALLQARYGYRMGNNEVVDLMIYDALLDPYTKRHMGELTEETIKKVGVTREEQDDYAERSYKLSQKAVESGIFDEEVVPVVKKTKKGDIVVDKDEEPFRVNFEKLRQLRPVFVKDGTITAGNASTINDGAACLLLASEDAVKKYNLKPIGRLVAYATNSIHPDEFSLAPVGAIEKVCEKAGLKLDDIDLFEINEAFAAVVLFAVKKLNLPLDKVNVNGGAVSIGHPVGASGGRLAVTLLKEMQRRNAKYGLATLCIGGGEAVSAIFERV

SEQ ID NO: 60 - protein, 402 aa, Rubrobacter xylanophilus
MSFGNGNGREVVISTPLRTAIGTFGGSLRDVPATELGATVGREVISRSGVDPERVDQVVVGNILSAGQGMNPARQVGIKSGLPVEAPAMTLNRMCGSGLQAIVSAAQEIALGDAEVVMAGGIENMDQAPFLLPKGRYGYRMGMPKADLLDHMVYDGLWDIFNDYHMGMTAENVAERYGVSREDSDAYAVRSHQRAARAIAEGYFDEQIVPVEVRQKKETVKFTRDEHVRENATLEGLARLKPVFKRDGGTVTAGNASGINDGAAMMLVSSARKAEELGLPVAGRLVSAAVAGVDPAIMGVGMVPASRAALKKAGLSIEDMDVVEANEAFASIAVTVGRELKVPEEKLNPLGGAVALGHPIGATGAILTVKILHELARTGGRYGLVTLCIGGGMGIAAIFERV

SEQ ID NO: 61 - DNA, 1179 bp, Deferribacter desulfuricans
atgcgggatgtctttgtagtcgaggggctgcgtacgccattcggaagctttggcggctcactgtcggatgtccatccggccgttttagctgcggacgtgatcaaaaagcttttagaaaaaacagaagtgaaaccggatgacatcgatgaagtcatcttgggacaggtgctcacaggcggatttggccaagccccagcccgtcaagctatgcgttacgcgggcctgttagacaaggttcatgctatgacgatcaataaagtttgcggctctggcttgaaggctttaatgctcggggcccaaagcattatgttgggcgattcagatctcgccatcgtcggaggcatggagaacatgagcatggcgccgtatgctttgctgcaggctcgttacggctatcgcatgggcaacaacgaggtggtggatttaatgatctatgatgcactgctcgacccgtataccaaacgccatatgggtgagttgacggaagaaacgatcaaaaaagtcggcgtgacccgcgaagaacaggatgactatgcggaacgcagctacaaactcagccaaaaagcagtggaatcaggcatctttgacgaggaagtggttcctgtggtcaaaaaaacaaaaaagggcgatattgtcgtcgataaagatgaagaaccgttccgggtcaactttgagaaactccggcagttacgcccggtcttcgtaaaagacggcaccattacagcgggtaatgcctcgaccattaacgatggtgccgcctgccttctcttggcctcagaagacgccgtcaagaaatataaccttaaaccaattggccgcttggtagcctatgccacgaattcgattcatccagacgagttcagcctcgcgccggtgggcgcgattgaaaaggtgtgtgaaaaagcgggcttaaaattagatgacattgacttatttgaaatcaatgaggcctttgctgcggtcgtcttgtttgctgtaaaaaaactcaatttgccgttagataaagtcaacgttaatggtggagcagtcagcattggccacccggtgggcgcgtcgggtggccggttagccgtcacgctgttgaaagaaatgcagcgccgtaacgcaaagtatggcttagccaccttgtgcatcggaggtggtgaagccgtgagcgcgattttcgagcgcgtctaatga

SEQ ID NO: 62 - DNA, 1212 bp, Rubrobacter xylanophilus
atgtcgtttggaaacggaaatggacgcgaagtcgtgatttcgacgccgttacgcaccgcgattggcacgttcggtgggtcgctccgcgacgttcctgcgacggagttgggtgcaacagtcgggcgtgaggtcatctcacgctccggcgttgatccggaacgcgtagaccaagtcgtggtaggaaacatcttgtcggcaggccaaggtatgaacccagcgcgccaagtcgggatcaagagcggcttgccagtcgaggccccagctatgacccttaaccgcatgtgtggatcgggcctccaagctattgtgtccgcggcgcaggagatcgcgctgggggatgcagaagttgtgatggctggcggaattgaaaatatggaccaagctccatttctgttgccgaaaggtcggtatggataccgtatgggtatgccaaaagctgacttattggatcatatggtctacgacggactgtgggatatcttcaacgattaccacatgggcatgacggccgagaatgttgccgaacgttatggtgtctcacgtgaagatagcgacgcatacgcagtgcgcagccatcagcgcgccgcgcgtgctattgcggaagggtattttgacgagcaaattgtccctgtggaggtgcgccagaagaaagaaacggtaaaattcacacgggatgagcacgttcgtgaaaacgcgacgttggaaggtcttgcccggttaaaaccagtctttaaacgcgacggaggcacggtcaccgccggaaacgcctcgggcattaacgatggcgcagccatgatgttggtctcaagcgctcgcaaggccgaagagttgggcttgccggtcgcgggtcgcctggtgtctgcggccgttgccggggtggatccggctattatgggggtcggcatggtgccggcatcacgcgctgctttaaaaaaagcaggcctttctattgaagacatggacgtcgtcgaggccaatgaagcctttgctagcatcgctgtcacggtagggcgtgaattgaaagttccggaagagaaacttaacccgctgggcggagcggtcgcgttgggtcatccgatcggcgccacgggagctatcctcacggtgaaaatcttgcatgagcttgcacgcacaggcggccgttatgggctggtaacgttgtgcatcggaggtggcatgggtatcgctgcaatcttcgaacgcgtgtaatga

SEQ ID NO: 63 - DNA, 1188 bp, Chloroflexus aurantiacus J-10-fl
atgagcgagaagcgagaggtcgtggtgctcagcggcgtgcgcacggccattggcacatttggtggtagtttgaaggatattccgccaaccgaattggcggcactggttacccgtgaagcagttgcccgctctggcctgcaaccaaacgaaatcggtcatgtggtcttcgggcacgtgatcaataccgaaccgcatgatatgtatctggctcgctatgcggcggtacgcggcggtctgtcggtagagactccagccctaacgcttaaccggctctgcggtagtgggttgcaggctatcgtgtcggcggcccaatacattttgcaaggtgatgctgaagcagccgttgccggtggtgccgagtgtatgagccgtggcccgtacagcttgccggccatgcgtttcggtgcccgtatgaatgattcaaaggtcgtcgatatgatggtcggtgccctaaccgacccgtttgacgattgccatatgggggtaactgccgaaaacgtggcggcaaagtggggaatcagccgcgaagatcaggatcaactggcttacgagagccatatgcgcgcagcgcgggcgattgacgaaggacgtttcgccaatcagatcgtgccggttgagattaaggtcaagggtggtaccgcccaattcatggttgatgaaggggtacgccgcgatacgaccatcgacaagctggccaagctccgcccggtgtttctgaaggatggttcggtgacggccggcaatgcttcgagcatcaatgatgcagcagcggcggtagtgctgatggatcgggccaccgctgagcggcgtggctacaagccgctggcgcgtctggtcggttacagccacgcagccgttgagccaaagtatatggggattggcccggtaccggctgtacgacgcctgcttgagcgcaccggcttgcgcatcagtgatattgatcttttcgaggtcaacgaagcttttgcagcgcaggcgctggctgtgatccgcgatctggagttgccgcctgatcgcacgaatcccaatggtagtggtatctccctcggtcacccgattggcgccaccggctgtattttgaccgtcaaagcaattcacgaactacaccgcaccggtggccgttatgccctggtcacgatgtgtatcggtggtgggcaggggattgctgcgatcttcgagcgaatgtag